(19) United States
(12) Patent Application Publication
(10) Pub. No.: US 2007/0160628 A1
(43) Pub. Date: Jul. 12, 2007

(54) Stabilized virus-like particles and epitope display systems

(76) Inventors: Ashley J. Birkett, Boston, MA (US); Edward P. Gamson, Highland Park, IL (US)

Correspondence Address: Edward P. Gamson, Suite 2200, 120 S. Riverside Plaza, Chicago, IL, 60606-3945, US

(21) Appl. No.: 11/507,083

(22) Filed: Aug. 18, 2006

Related U.S. Application Data
(60) Provisional application No. 60/713,148, filed on Aug. 31, 2005.

(51) Int. Cl. C12Q 1/68; A61K 39/12

(52) U.S. Cl. 424/204.1; 435/6

(57) ABSTRACT

A chimeric polypeptide is disclosed that comprises: i) a first portion that self-assembles into an organized, repetitive, supramolecular structure that contains at least about 9 subunits, covalently linked to ii) a second polypeptide portion that comprises a peptide having a length of about 15 to about 80 amino acid residues. The second portion peptide self-assembles to form parallel multimers. A contemplated chimeric polypeptide forms a particle that is more stable than is a particle formed from a first polypeptide that is otherwise identical in sequence, but lacks the covalently linked self-binding peptide sequence of the second polypeptide portion.

### CROSS-REFERENCE TO RELATED APPLICATION

This application claims benefit of provisional application Ser. No. 60/713,148 that was filed on Aug. 31, 2005.

### TECHNICAL FIELD

The present invention relates to the intersection of the fields of immunology and polypeptide engineering, and particularly to a chimeric polypeptide and supramolecular assemblages thereof that are engineered for reduced nucleic acid binding, enhanced stability and display of immunogenic epitopes.

### BACKGROUND OF THE INVENTION

Although virus particles often consist of one or a few different polypeptides, they are able to trigger much stronger immune responses than their isolated components. For B cell responses, it is known that one important factor for the immunogenicity of virus particles can be the repetitiveness and order of surface epitopes. The surfaces of most virus particles include polypeptides arranged in a regular, symmetric quasi-crystalline manner that displays a regular array of epitopes, such that epitope-specific immunoglobulins on B cells efficiently cross-link [Bachmann et al., (1996) Immunol. Today 17:553-558]. This cross-linking of surface immunoglobulins on B cells is a strong activation signal that directly induces cell-cycle progression and the production of IgM antibodies. Further, such triggered B cells are able to activate T helper cells, which in turn induce a switch from IgM to IgG antibody production in B cells and the generation of long-lived B cell memory—the goal of any vaccination [Bachmann et al., (1997) Ann. Rev. Immunol. 15:235-270]. Viral structure is even linked to the generation of anti-antibodies in autoimmune disease and as a part of the natural response to pathogens [see Fehr et al., (1997) J. Exp. Med. 185:1785-1792]. Thus, antigens on viral particles that are organized in an ordered and repetitive array are highly immunogenic since they can directly activate B cells.

In addition to strong B cell responses, viral particles also induce a cytotoxic T cell response, another crucial arm of the immune system. Cytotoxic T cells are particularly important for the elimination of non-cytopathic viruses such as HIV or hepatitis B virus and for the eradication of tumors. Cytotoxic T cells do not recognize native antigens but rather recognize their degradation products in association with MHC class I molecules [Townsend et al., (1989) Ann. Rev. Immunol. 7:601-624]. Macrophages and dendritic cells are able to take up and process exogenous viral particles but not their soluble, isolated components and present the generated degradation product to cytotoxic T cells, leading to their activation and proliferation [Kovacsovics-Bankowski et al., (1993) Proc. Natl. Acad. Sci. 90:4942-4946; Bachmann et al., (1996) Eur. J. Immunol. 26:2595-2600].

Several new vaccine strategies exploit the inherent immunogenicity of viruses. Some of these approaches focus on the particulate nature of the virus particle; for example see Harding et al., (1994) J. Immunol. 153:4925-33, which discloses a vaccine consisting of latex beads coated with antigen; Kovacsovics-Bankowski et al. (1993) Proc. Natl. Acad. Sci. 90:4942-4946, which discloses a vaccine consisting of iron oxide beads and antigen; U.S. Pat. No. 5,334,394 to Kossovsky et al., which discloses particles coated with antigen; U.S. Pat. No. 5,871,747 to Gengoux-Sedlik, which discloses synthetic polymer particles carrying on the surface one or more polypeptides covalently bonded thereto; and a particle with a non-covalently bound coating, which at least partially covers the surface of that particle, and at least one biologically active agent in contact with said coated particle (see, e.g., WO 94/15585).

Virus-like particles (VLPs) are structures built in an organized and geometrically regular manner from many polypeptide molecules of one or more types. Being comprised of more than one molecule, VLPs can be referred to as being supramolecular. VLPs lack the viral genome and, therefore, are non-infectious. VLPs can be produced in large quantities by heterologous expression and can be easily purified. The geometry of a VLP typically resembles the geometry of the source virus particle, so for most cases discussed below, the geometry has icosahedral or pseudo-icosahedral symmetry. VLPs are being exploited in the area of vaccine production because of their structural properties, their ease in large scale preparation and purification, and their non-infectious nature.

Examples of VLPs include the self-assemblages of capsid or nucleocapsid polypeptides of hepatitis B virus [WO 92/11291, Ulrich, et al., (1998) Virus Res. 50:141-182], measles virus [Warnes, et al., (1995) Gene 160:173-178], Sindbis virus [Tellinghuisen et al., (1999) J. Virol. 73:5309-5319], hepatitis C virus [Baumbert (1998) J. Virol. 72:3827-3836] rotavirus [U.S. Pat. No. 5,071,651 to Sabara, et al. and No. 5,374,426 to Sabara et al.], foot-and-mouth-disease virus [Twomey, et al., (1995) Vaccine 13:1603-1610], Norwalk virus [Jiang et al., Science (1990) 250:1580-1583; Matsui et al., J. Clin. Invest. (1991) 87:1456-1461], retroviruses [WO 96/30523; U.S. Pat. No. 6,602,705 to Barnett et al.], the retrotransposons [e.g., the Ty polypeptide p1, in Al-Khayat et al., (1999) J. Mol. Biol. 292:65-73 and U.S. Pat. No. 6,060,064 to Adams et al.], and human papilloma virus [WO 98/15631]. Plant-infecting virus capsid polypeptides also self-assemble into VLPs in vitro and in vivo [see, e.g., potyvirus, Jagadish et al., (1991) J. Gen. Virol. 72:1543-1550., alfalfa mosaic virus, Yusibov et al., (1996) J. Gen. Virol. 77:567-573]. The structural subunits of virus particles and their cognate VLPs are referred to hereinafter as “capsid” polypeptides.

There are numerous examples where the capsid polypeptide self-assembles into structures other than the usual virion structures. For example, rotavirus [Lepault et al., (2001) EMBO J. 20:1498-1507], papillomavirus and polyoma virus [Schwartz et al., (2000) Virology. 268:461-470] capsid polypeptides each form various particles and tubular structures, and some capsid polypeptides even assemble into organized two-dimensional arrays depending on assembly conditions [Lane (1981) Handbook of Plant Virus Infections and Comparative Diagnosis, ed. Kurstak, E. (Elsevier/North-Holland, Amsterdam) pages 334-376]. Nevertheless, each of these assemblages exhibits geometrical regularity in the arrangement of the capsid polypeptides, as is readily observable by x-ray diffraction analysis, electron microscopy and other methods. Although these assemblages of virus capsid polypeptides typically comprise greater than about thirty subunits, smaller VLP assemblages are derived from negative-stranded RNA viruses. Ring-shaped VLPs have been described that comprise approximately 9 copies of the nucleocapsid protein of the influenza A virus [Portela (2002) J. Gen. Virol. 83:723-734] and ring shaped VLPs comprising 10±1 copies of the rabies nucleoprotein have been described by Iseni (2002) J. Gen. Virol. 79:2909-2919. The capsid polypeptide of the positive-sense RNA tobacco mosaic virus self-assembles into VLPs that are two-layered disks comprised of 34 copies of the capsid polypeptide (17 copies per layer). [Bhyravbhatla (1998) Biophys. J. 74:604-615]

Fusion of heterologous epitopes to capsid polypeptides that make VLPs bearing those epitopes is described above and is well known in the art. Other multimeric supramolecular structures that display epitopes have also been disclosed that are also intended for use as a vaccine. For example, in WO 00/69907, Hill et al. disclose use of chaperonin heptamers and double heptamers, e.g. GroES and GroEL from E. coli and homologues, as a protein scaffold into which heterologous polypeptides can be inserted. That system provides another supramolecular structure for polyvalent presentation of inserted polypeptides, but neither higher order structures nor stabilizing features were disclosed or suggested beyond the native subunit interactions and arrangements.

Rod-shaped viruses (e.g., tobacco mosaic virus), flexuous viruses (e.g., the potato viruses X and Y; Ebola virus) and flexuous virus substructures (e.g., the nucleocapsids of HIV, influenza A and rabies viruses) are helical; they each posses radial symmetry. Therefore geometrically regular assemblages and structures that are neither spherical nor icosahedral are expressly included in the term ‘virus-like particle’ (VLP). Indeed, tubular structures, like icosahedral structures, have been demonstrated to be effective and protective immunogens in vivo by Ghosh et al., (2002) Virology 302:383-392.

Full-length virus-derived capsid polypeptides typically bind nucleic acids present in cells expressing the capsid polypeptide. This reflects the capsid polypeptide's natural biological functionality to associate with, package and deliver the viral genome. Following self-assembly, VLPs consisting of unaltered capsid polypeptides typically include nucleic acids present in the cells in which the capsid polypeptide is expressed, including the capsid polypeptide messenger RNA as well as other cellular nucleic acids [see for example, Iseni et al., (1998) J. Gen. Virol., 79:2909-2919; Krol et al., (1999) Proc. Natl. Acad. Sci. 96:13650-13655; and Yu et al., (2001) J. Virol. 75:2753-2764].

Nucleic acid binding can be undesirable where a VLP is to be used as a vaccine (as opposed to use as a gene delivery system, as disclosed in U.S. Pat. No. 5,869,287 to Price et al.) because one would not want to introduce foreign DNA or RNA into the animal or person to be immunized. Such foreign nucleic acids are often carried along following heterologous expression.

Nucleic acid binding is often associated with a discrete segment of the capsid polypeptide that can be deleted to give a capsid polypeptide with reduced or substantially no nucleic acid binding. [e.g., Choi et al., (2000) Virology 270:377-385.] In some cases, however, deletion of the nucleic acid binding domain of the capsid polypeptide leads to particle instability or prevents particle assembly because core-nucleic acid interactions contribute to the stability of the particle [Schmitz et al., (1998) Virology 248:323-331; in general, see Harrison (2001) “Principles of Virus Structure” in Fields' Virology, 4^{th} ed. Lippincot, [Philadelphia] pages 53-86, and the citations therein]. Indeed, structural contributions of nucleic acid to virus particles are the rule rather than the exception, so a compensatory feature is often required to stabilize VLPs in the absence of nucleic acids.

VLPs designed to display immunogenic epitopes are known in the art, and have been designed from the structural polypeptides of many types of viruses including viruses that infect bacteria (bacteriophages), plants and animals, including human-infecting viruses. Wolf et al. in WO 96/30523 have described the use of altered pr55 Gag of HIV-1 to act as a non-infectious retroviral-like particulate carrier for the presentation of immunologically important epitopes. Nagesha et al. have explored the use of caliciviruses as epitope carriers in (1999) Arch. Virol. 144:2429-2439. Porta et al. reviewed the use of VLPs derived from plant-infecting viruses as epitope carriers in (1998) Rev. Med. Virol. 8:25-41. Brown et al. (2002) Intervirology 45:371-380 describe the use of bacteria-infecting viruses as epitope carriers. A non-exclusive list of exemplary peptide epitopes is provided in Table 1 hereinafter.

However, such engineered or altered capsid polypeptides can have reduced capacity for self-assembly [see e.g., Schmitz et al., Virology (1998) 248:323-331]. VLPs comprised of chimeric hepatitis B (HB) capsid polypeptide bearing internal insertions often appear to have a less ordered structure, when analyzed by electron microscopy, compared to VLPs that lack inserted heterologous epitopes [Schödel et al. (1994) J. Exp. Med., 180:1037-1046]. In some cases, the attachment of heterologous epitopes to C-terminally truncated HB capsid polypeptide has such a dramatic destabilizing affect that hybrid VLPs cannot be recovered following capsid polypeptide expression [Schödel et al. (1994) Infect. Immunol., 62:1669-1676]. Thus, many engineered HB VLPs are so unstable that they do not form, fall apart during purification to such an extent that they are unrecoverable or they show very poor stability characteristics, making them problematic for vaccine development. This instability has also been demonstrated by Fehr et al. for the bacteriophage Q-beta [(1998) Proc. Natl. Acad. Sci. 95:9477-9481]. There, a particular epitope insertion that prevented particle assembly also prevented immunogenic recognition of the inserted epitope, reinforcing the importance of VLP stability in vaccine design.

Ulrich et al., Adv. Virus Res., vol. 50 (1998) Academic Press, (New York) pages 141-182 report a loss of particle stability on C-terminal truncation and insertion of foreign sequences for the hepatitis B capsid polypeptide (HBc). As a consequence, a structural feature that promotes or reconstitutes VLP stability, while abrogating the nucleic acid binding ability of full-length capsid polypeptide and tolerates attachment of a heterologous epitope would be highly beneficial in vaccine development. This is well appreciated for the exemplary antigen delivery systems that use the capsid polypeptide of hepatitis B and other hepadnaviruses. In that same paper, Ulrich et al. note three potential problems to be solved for use of those chimers in human vaccines. A first potential problem is the inadvertent transfer of nucleic acids in a chimer vaccine to an immunized host. A second potential problem is interference from preexisting immunity to HB. A third possible problem relates to the requirement of reproducible preparation of intact chimer particles that can also withstand long-term storage.

Pumpens et al., (1995) Intervirology, 38:63-74 reported that VLPs formed from C-terminal truncated HB capsid polypeptides are less stable than VLPs formed from the full length proteins. C-Terminal truncated HBc chimers containing an insert to the HBc sequence often appear to have a less ordered structure, when analyzed by electron microscopy, compared to particles that lack heterologous epitopes [Schodel et al., (1994) J. Exp. Med., 180:1037-1046]. In some cases the insertion of heterologous epitopes into C-terminally truncated HBc particles has such a dramatic destabilizing affect that hybrid particles cannot be recovered following heterologous expression [Schodel et al. (1994) Infect. Immunol., 62:1669-1676]. Thus, many chimeric HBc particles are so unstable that they fall apart during purification to such an extent that they are unrecoverable or they show very poor stability characteristics, making them problematic for vaccine development.

Reduced stability is well appreciated as a major obstacle to practical use of engineered VLPs as vaccines in general, and solutions have been sought. For example, Peabody (1997) Arch. Biochem. Biophys. 347:85-92 showed that self-assembly of bacteriophage MS2 capsid polypeptide bearing an inserted peptide was defective. In that particular case, however, fusing two modified capsid polypeptides stabilized intra-dimer capsid interactions as self-assembly was restored by genetically (and hence, covalently) linking two such capsid polypeptides. This solution is not generalizable because of the diversity of VLP structures and protein interactions; not all VLPs are constructed of dimers, and so the required inter-capsid polypeptide interactions there would be disrupted by such protein-protein fusions. Even where VLPs are built from dimers, the structural limitations imposed by such a head-to-tail covalent bond can prevent proper assembly.

Self-binding peptide sequences are present in the so-called leucine zipper proteins. Exemplary leucine zipper sequences are present in the proteins known as GCN4, jun, fos, c-Myc, Max and C/EBP. Of those sequences, the GCN4 sequence has been well studied and shown to form parallel dimers, whereas mutated forms of GCN4 have been shown to form parallel dimers, trimers and tetramers [Harbury et al., (1993) Science, 262:1401-1407]. Such peptides, via their self-assembly, have been used to functionally replace native self-interacting protein domains and/or inter-protein interactions.

In one case, the role of a highly conserved cytoplasmic domain in ion channel assembly was identified because it was functionally replaced by a leucine zipper [Zerangue et al., (2000) Pro. Nat. Acad. Sci. 97:3591-3595]. In another case, a leucine zipper peptide could substitute, in deletion variants of the HIV p55 (gag) structural polyprotein, for assembly activity associated with the nucleocapsid and its interactions with capsid proteins; that is, for interactions by which the outer capsid protein is organized by the inner nucleocapsid protein [see, e.g., Accola et al., (2002) J. Virol. 74:5395-5402 and Zhang et al., (1998) J. Virol. 72:1782-1789]. More recently, the N-terminal helical domain of Sindbis nucleocapsid protein was functionally replaced by a dimer-forming, but not a trimer-forming, leucine zipper in live viruses. [Perera et al., (2003) J. Virol. 77:8345-8353]

All of the above reports used leucine zippers as tools for functionally defining the activities of the domains that they replaced. These papers neither disclose nor suggest the use of self-binding peptides to stabilize VLPs as such, nor do they disclose or suggest that VLPs so stabilized could be useful for epitope delivery and/or immunization.

The use of other self-binding peptides to create relatively small, artificial supramolecular structures is also known in the art. Thus, ThØgerson et al. disclose a system in WO 98/56906 for creating trimers of fusion polypeptides bearing structural elements derived from the tetranectin protein family. This system creates stable homo- or hetero-dimers and trimers, but does not create higher order structures. Further, the tetranectin trimerization domain is rich in lysines that can exacerbate rather than reduce nucleic acid binding.

In WO 96/37621, Pack et al. disclose the use of mammalian p53, platelet factor 4, COMP, or histone-derived multimerization domains to create novel homo- and hetero-tetramers and pentamers of fusion polypeptides with the general structure (functional domain 1)-(multimerization domain)-(functional domain 2). However, that publication does not suggest the use of multimerization domains to stabilize higher order or pre-existing structures. Further, these structures (excepting COMP) have an anti-parallel arrangement, that is, the multimerization domains lay head-to-tail relative to one another in the multimer.

In WO 02/74795, De Filette et al. disclose a recombinant, multivalent influenza vaccine consisting of a leucine-zipper self-assembly domain fused to an influenza antigenic polypeptide. There, an antigen, derived from a naturally occurring oligomeric protein complex, is fused to an oligomerization domain. No suggestion was made that such oligomerization domains might find utility stabilizing the source protein complex or any higher order supramolecular structures such as VLPs, nor are structures larger than tetramers discussed. Rather, the oligomerization domain is said to drive the oligomerization and control the degree of oligomerization. This is also the case for each of the other disclosures discussed here.

Another exemplary self-binding peptide is the M2 protein of the influenza A virus. The M2 protein of the influenza A virus is a small ion channel protein that self aggregates into homo-tetramers at membrane surfaces. The tetramers are stabilized by non-essential intermolecular disulfide bonds that form between cysteines at positions 17 and 19 [Holsinger et al., (1991) Virology 183:32-43]. Although this region of the M2 protein has found use as an antigen in various recombinant influenza vaccines when bound to VLPs or self-binding peptides [see e.g. Neirynck et al., (1999) Nat. Med., 5(10):1157-1163 and WO 99/07839, and WO 02/74795, De Filette et al. and citations therein] it has not been previously reported as useful to stabilize VLPs and, quite unexpectedly, this stabilization did not require disulfide bonds at positions corresponding to positions 17 and 19.

Self-binding peptides need not be derived from known or natural proteins, but can be identified de novo. [Zhang et al., (1999) Curr. Biol. 9:417-420.] Self-assembling peptides can also be designed to assemble in solution when an appropriate metal ion is introduced. [Ghadiri et al., (1992) J. Am. Chem. Soc. 114:4000-4002] For example, the N-acylated (indicated by “Ac-” in the sequence below) peptide having the sequence using single letters

(SEQ ID NO:1)

Ac-G-L-A-Q-K-L-L-E-A-L-Q-K-A-L-A-CONH₂

self-assembles into a parallel tetra-helix in the presence of an appropriate metal ion. The N-acylated peptide having the sequence

(SEQ ID No:2)

Ac-G-E-L-A-E-Q-K-L-E-Q-A-L-Q-K-L-A-CONH₂

self-assembles into a parallel tri-helix [U.S. Pat. No. 5,408,036 to Ghadiri].

Other self-binding peptides well known in the art can be chosen for their specific properties, including expected immunogenicity, source organism, preferred multimeric state, and binding strength. For example, self-binding peptides from human proteins can minimize the immune reaction to the self-binding peptide when administered a vaccine (or presents other attached epitopes) to a human.

As is disclosed hereinafter, the present invention provides one solution to problems associated with VLP-derived vaccines, and provides such vaccines with substantial freedom from inadvertent nucleic acid binding and instability, while retaining the benefits of VLP usage such as ease and flexibility of production, adjuvant-like immunogenic enhancement.

### BRIEF SUMMARY OF THE INVENTION

Stability of a supramolecular assemblage such as a virus-like particle (VLP), and hence utility of materials, e.g. as vaccines, can be substantially improved by the addition of self-binding peptides to the viral protein (polypeptide) chain subunits. This solution to stability (VLP) instability is generalizable and can be utilized to stabilize diverse VLPs derived from viruses that infect animals, plants, fungi and bacteria.

The contemplated solution to VLP stabilization is also useful to stabilize other large, repetitive and symmetrical supramolecular assemblages. Examples of non-viral VLPs (supramolecular assemblages) that can be stabilized include known 60-subunit icosahedral enzyme complexes of pyruvate dehydrogenases of eukaryotes and prokaryotes [Lessard et al. (1998) EMBO J. 258: 491-501; Stoops et al. (1997) J. Biol. Chem. 272: 5757-5764; Wagenknecht et al. (1991) J. Biol. Chem. 266: 24650-24656] and prokaryotic lumazine synthases. [Fischer et al. (2003) Eur. J. Biochem. 270:1025-1032; Mortl et al. (1996) J. Biol. Chem. 271:33201-33207] Like virus-derived VLPs, these icosahedral particles have utility for epitope presentation and delivery in vaccines or other immunogens.

The phrase “supramolecular assemblage” in its various grammatical forms is used herein to encompass both virally-related (or -derived) VLPs; i.e., VLPs encoded by a viral genome or humanly altered viral genome, and similar particles of non-viral origin. The term “VLP” is usually used for those particles that are formed from viral genes or humanly altered viral genes, but is also used interchangeably used with supramolecular assemblage because of its familiarity and ease of usage.

The present invention contemplates a chimeric supramolecular assemblage (or VLP) that is comprised of a plurality of polypeptide subunits that contain an attached self-assembling peptide sequence. Thus, a contemplated chimer comprises (i) a first polypeptide portion that self-assembles into an organized, repetitive, supramolecular structure that contains at least about 9 subunits, covalently linked to (ii) a second peptide portion that comprises a heterologous peptide having a length of about 15 to about 80 amino acid residues. The second portion peptide, when not so covalently linked to the first portion, self-assembles to form parallel multimers, as is discussed hereinafter. A contemplated chimeric polypeptide forms particles that are more stable than are a particles formed from a first polypeptide that is otherwise identical in sequence, but lacks the covalently linked self-binding peptide sequence of the second polypeptide portion. That stability can be assayed using analytical size exclusion chromatography elution. A contemplated chimeric supramolecular assemblage (VLP) upon immunization in a mammalian host preferably induces immunogenicity toward one or more of the supramolecular assemblage polypeptide sequences, a heterologous polypeptide fused to the supramolecular assemblage polypeptide sequence or an attached hapten.

More specifically, the present invention contemplates a supramolecular assemblage comprised of chimeric polypeptides that themselves comprise: (i) a first polypeptide portion that self-assembles into an organized, repetitive, supramolecular structure that contains at least about 9 polypeptide subunits, and (ii) a covalently linked second peptide portion that comprises a polypeptide (that is preferably heterologous to the polypeptide of the first portion) having a length of about 15 to about 80 amino acid residues. The second portion peptide, (a) when present as an N-acetylated peptide self-assembles to form parallel multimers in aqueous PBS solution at pH 7.0 and at a concentration of about 10 millimoles/liter, or (b) self assembles to form parallel multimers when present in solution at a concentration of at least ten micromoles/liter in the presence of a five-fold molar excess of a predetermined multivalent metal ion, or (c) is the self-assembling, extracellular domain of the influenza A M2 polypeptide, with the proviso that the second portion is other than that extracellular M2 domain, which contains zero, one or two cysteines, N-terminally bonded to a first portion that is a HBc polypeptide. Thus, in a preferred embodiment, the second portion peptide is as defined in one or the other of (a) or (b) or (c). Second portion peptides as defined in (a) and (c) are particularly preferred. When administered to a mammal in an effective amount, the chimeric polypeptide preferably elicits an immune response toward one or more of the VLP polypeptide sequence, a polypeptide heterologous to the VLP polypeptide sequence or an attached hapten.

In a preferred embodiment, this invention contemplates a chimeric polypeptide that comprises: (i) a first portion that has the sequence of all or a portion of a viral capsid protein or is a derivative of a virus capsid protein (as defined hereinafter), that is covalently linked to (ii) a second portion that comprises a peptide having a length of about 15 to about 80 amino acid residues. The peptide of the second portion, (a) when present as an N-acetylated peptide self-assembles to form parallel multimers in aqueous PBS solution at pH 7.0 and at a concentration of about 10 millimoles/liter, or (b) self assembles when present in solution at a concentration of at least ten micromoles/liter in the presence of a five-fold molar excess of a predetermined multivalent metal ion, or (c) is the self-assembling, extracellular domain of the influenza A M2 polypeptide, with the proviso that the second portion is other than that extracellular M2 domain, which contains zero, one or two cysteines, covalently bonded to the N-terminus of a first portion that is a HBc polypeptide. When administered to a host animal in an effective amount, the chimeric polypeptide preferably elicits an immune response. In some embodiments the first portion is derived from or is similar to a capsid polypeptide from the group of viruses including animal-infecting viruses, plant-infecting viruses, fungus-infecting viruses, and bacteria-infecting viruses.

In some embodiments, the invention comprises a chimeric polypeptide with a linker residue, a heterologous epitope or both included in the first polypeptide portion. In other embodiments, the invention is directed to the organized, self-assembled supramolecular particle that forms from the chimeric polypeptide.

### BRIEF DESCRIPTION OF THE DRAWINGS

In the drawings forming a part of this disclosure,

FIG. 1, shown in two panels as FIG. 1A and FIG. 1B, provides an alignment of six published sequences for mammalian HBc proteins from six viruses. The first (SEQ ID NO:3), human viral sequence is of the ayw subtype and was published in Galibert et al. (1983) Nature, 281:646-650; the second human viral sequence (SEQ ID NO:4), of the adw subtype, was published by Ono et al. (1983) Nucleic Acids Res., 11(6): 1747-1757; the third human viral sequence (SEQ ID NO:5), is of the adw2 subtype and was published by Valenzuela et al., Animal Virus Genetics, Field et al. eds., Academic Press, New York (1980) pages 57-70; the fourth human viral sequence (SEQ ID NO:6), is of the adyw subtype that was published by Pasek et al. (1979) Nature, 282:575-579; the fifth sequence (SEQ ID NO:7), is that of the woodchuck virus that was published by Galibert et al. (1982) J. Virol., 41:51-65; and the sixth mammalian sequence, (SEQ ID NO:8), is that of the ground squirrel that was published by Seeger et al. (1984) J. Virol., 51:367-375.

FIG. 2 schematically shows DNA that encodes a version of influenza A M2 external domain (positions 2-24), where the cysteines were mutated to serines was used (CV-1895, V7.M2e(2C>2S) (SEQ ID NO:9) cloned into the EcoRI and HindIII sites at the C-terminus of an engineered, truncated HBc gene containing the first 149 HBc residues (HBc149).

FIG. 3 shows an analytical size exclusion chromatography elution profile for particles designated CV-1048. Assembled particles elute at about 8 mL, whereas lower order structures elute in subsequent peaks. Samples were run on Superose® 6 HR (Pharmacia) in 20 mM sodium phosphate, pH 6.8 and 0.02% sodium azide Absorbance at 280 nm is shown on the ordinate and volume in milliliters (mL) is shown on the abscissa.

FIG. 4 shows an analytical size exclusion chromatography elution profile as show in FIG. 3 for CV-1895 particles. Here, the stabilized particles elute at about 7 minutes and substantially no lower order structures were observed.

FIG. 5, adapted from U.S. Pat. No. 6,231,864, illustrates a reaction scheme (Scheme 1) that shows two reaction sequences for (I) forming an activated carrier for pendently linking a hapten to a virus-like particle (VLP) using sulpho-succinimidyl 4-(N-maleimidomethyl)-cyclohexane 1-carboxylate (sulpho-SMCC), and then (II) linking a sulfhydryl-terminated (cysteine-terminated) hapten to the activated carrier to form a conjugate particle. The VLP is depicted as a box having a single pendent amino group (for purposes of clarity of the figure), whereas the sulfhydryl-terminated hapten is depicted as a line terminated with an SH group.

The present invention has several benefits and advantages.

One benefit of the invention is that virus-like particles formed from chimeric capsid polypeptides including self-binding peptide portions are more stable on storage in aqueous compositions than are particles of similar sequence that lack self-binding peptide portions.

An advantage of certain embodiments of the invention is that some chimeric capsid polypeptides exhibit the self-assembly characteristics of a native virus-like particle, and exhibit only partial to substantially none of the nucleic acid binding of those native particles. Because icosahedral enzyme complexes are not adapted to encapsidate a viral genome, VLPs derived from icosahedral enzyme complex subunits lack nucleic acid binding without requiring special modification.

The utility of this invention is enhanced by the availability of at least dimer-, trimer-, and pentamer-forming self-binding peptides. Icosahedral viruses and VLPs have 2-, 3-, and 5-fold symmetries, and hence VLP stabilization can be optimized by enhancing 2-fold (dimer), 3-fold (trimer) and/or 5-fold (pentamer) capsid polypeptide interactions. It is noted that a tetramer has 2-fold symmetry and a hexamer has both 2- and 3-fold symmetry. Without wishing to be bound by theory, however, the 2-, 3-, and 5-fold symmetry of icosahedra affords the worker choice of stabilizing 2-, 3- or 5-fold intra-capsid polypeptide interactions, and vice versa. A non-exclusive listing of exemplary self-binding peptides is provided hereinafter in Table 2.

Another benefit of the present invention is that virus-like particles formed from chimeric capsid polypeptides can exhibit excellent B cell and T cell immunogenicities.

Another advantage is that VLPs of the present invention can be prepared in higher yield than are similar particles that lack the self-binding peptide portion.

A further benefit of the invention is that contemplated VLPs bearing attached epitopes are often far more immunogenic than are similar conjugates that lack a self-binding peptide portion. This enhanced immunogenicity is attributable to stabilized particles retaining their particulate nature for a longer period of time following introduction into an animal as an immunogen.

A further advantage of the invention is that capsid polypeptides derived from a wide range of viruses can be used. A still further benefit of the invention is that linkage of a self-binding peptide portion to a VLP sequence provides a generalized solution to problems of VLP instability.

Still further benefits and advantages of the present invention will be apparent to one skilled in the art from the disclosure that follows.

Definitions

EPITOPE An epitope is defined to include small molecules such as are generally known as haptens in the field of immunology (e.g., dinitrophenol, drugs such as nicotine or cocaine) as well as B-cell epitopes, T-cell epitopes, antigens, antigenic determinants, binding sites for monoclonal antibodies, peptides, oligosaccharides, nucleic acids, and other chemical and biological entities as known in the art to be subject to immune recognition, typically by binding to the paratope of an antibody or to a T cell receptor. The epitope is distinct from the capsid polypeptide and the self-binding peptide.

CHIMERIC POLYPEPTIDE A chimeric polypeptide is a polypeptide that does not occur in nature and typically includes one or more attached moieties such as other polypeptides, peptides, linker resides or moieties, and/or other molecules. In most contexts the term means a polypeptide comprising first and second polypeptide portions attached by a covalent bond. In many contexts the term further means one polypeptide from one source or genetic heritage attached to another from another source or genetic heritage attached by a peptide bond via expression of a genetically engineered protein coding sequence. However as is noted elsewhere, the art enables a skilled worker to synthesize a chimeric polypeptide through entirely non-biological in vitro chemical syntheses in whole or part.

VLP VLP is an abbreviation of for virus-like particle, and refers to ordered, repetitive supramolecular structures and complexes formed by self-assembly, usually of a viral capsid polypeptide. In some cases the VLP is not derived from a virus capsid polypeptide; i.e., does not have the amino acid sequence of a viral capsid protein, but is derived from (has the sequence of) another self-assembling polypeptide such as the pyruvate dehydrogenase E2 polypeptide and the lumazine synthase polypeptide. VLPs may or may not include any nucleic acid component. VLPs derived from viral capsid polypeptides comprise at least 9 capsid polypeptides. VLPs derived from other self-assembling polypeptides comprise at least about 30 subunits. VLP morphologies include icosahedra, particles with icosahedral or quasi-icosahedral symmetry, spherical, tubular and filamentous structures, and planar arrays. Like natural viruses, VLPs exhibit at least some regions with two-fold, three-fold, five-fold, or radial symmetry. Such local or total symmetry is readily observable by many techniques, including X-ray diffraction and electron microscopy with or without additional image analysis. VLP, as defined here, expressly includes morphologies and assemblages that are dissimilar to the virus or particle from which the capsid polypeptide is ultimately derived.

CAPSID POLYPEPTIDE A capsid polypeptide is a polypeptide that exhibits self-assembly into a VLP. A capsid polypeptide contemplated herein can be a natural polypeptide subunit of a virus particle, a polypeptide subunit of a non-viral particle (as discussed below), or a non-natural derivative. The structural protein subunits of virus particles and their cognate VLPs are comprised of capsid polypeptides. Capsid polypeptides can include additional moieties such as self-binding peptides, linker sequences or epitopes and therefore also be a chimeric polypeptide. A capsid polypeptide is distinct from and heterologous relative to an epitope and a self-binding peptide. A capsid polypeptide preferably does not itself elicit an immune response when attached to a heterologous epitope, but may nevertheless do so. The term “capsid polypeptide” is expressly intended to include both naturally occurring and artificial analogues and derivatives of a structural protein of a virus particle. Capsid polypeptide includes those peptides known in the art as nucleocapsid proteins and core proteins. Capsid polypeptides need not be the only polypeptide present either in the originating virus or in the VLP. Capsid polypeptide expressly includes other self-assembling polypeptides not of viral origin, such as the E2 subunit of the pyruvate dehydrogenase enzyme complex, and the lumazine synthase polypeptide, each of which form particles with icosahedral symmetry comprised of 60 subunits.

A derivative capsid polypeptide has an amino acid sequence that is at least about 80 percent identical to the sequence of a viral capsid polypeptide. Put differently, a contemplated capsid polypeptide can have up to about 20 percent of the amino acid residues substituted as compared to one or more homologous regions of a naturally occurring capsid polypeptide from which the derivative is derived. Where a sequence is truncated at the N- or C-terminus, or contains one or more additional residues or deletions within the sequence, those regions are not included in the calculation of percent substitutions because the change in sequence has no homologous sequence in the natural capsid polypeptide.

SELF-ASSEMBLY In the context of capsid polypeptides and VLPs, self-assembly refers to virion or VLP assembly, in vivo or in vitro, from the a capsid polypeptide (or analogue) with or without intervention of a human worker. Self-assembly also refers to the assembly of organized, repetitive supramolecular structures from other particle-forming chimeric polypeptides not of viral origin, such as lumazine synthase and the E2 subunit of the pyruvate dehydrogenase enzyme complex. In the context of self-binding peptides (discussed below), self-assembly refers to the association of individual chimeric polypeptides in solution into multimers or to form aggregates of at least about nine polypeptide subunits when present in solution. Chimeric polypeptides generally self-assemble as globular subunit entities that possess individual secondary and tertiary proteinaceous structures and come together to form a particulate entity comprised of at least nine subunits.

SELF-BINDING PEPTIDE A self-binding peptide is attached to a capsid polypeptide to stabilize intra-particle polypeptide interactions by inter-peptide interactions. A self-binding peptide includes those peptides that when present as an N-acetylated peptide spontaneously come together to form parallel multimers in aqueous PBS solution at pH 7.0 and a concentration of about 10 millimoles/liter; those peptides that self-assemble into parallel multimers when present in solution at a concentration of at least ten micromoles/liter in the presence of a five-fold molar excess of an acceptable multivalent metal ion, and the about 16 to about 24 amino acid residue extracellular domain of the influenza M2 protein identified in this disclosure as able to stabilize VLPs. The self-binding peptide is distinct from and heterologous relative to the chimeric polypeptide and an epitope, although that peptide may contain one or more epitopes and be included in the sequence of a chimeric polypeptide. The self-binding peptide preferably does not elicit an immune response, but may nevertheless do so. Self-binding peptides contemplated here form parallel multimers containing fewer than nine members and typically obtain whatever secondary and/or tertiary structure they may possess as multimers. In addition, self-binding peptides bind to each other without forming interpeptide covalent bonds.

VIRUS The use of the term “virus” and the taxonomy of viruses is according to the Seventh Report of the International Committee on Taxonomy of Viruses, van Regenmortel, M. H. V. ed. (San Diego) Academic Press 2000. As such, agents without extracellular counterparts or horizontal transmissibility are expressly included, as are the bacteriophages (e.g., the Ty 1 retrotransposon and the L-A virus of Saccharomyces cerevisiae each form particles in vivo that never leave the cell to infect other individual cells but are transmitted from parent to daughter).

RESIDUE and AMINO ACID RESIDUE The term “residue” is used interchangeably with the phrase amino acid residue. All amino acid residues identified herein are in the natural or L-configuration, unless specifically indicated as being in the D-form. In keeping with standard polypeptide nomenclature, [J. Biol. Chem., 243:3557-59 (1969)], abbreviations for amino acid residues are as shown in the following Table of Correspondence.

TABLE OF CORRESPONDENCE

| 1-Letter | 3-Letter | AMINO ACID |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| Z | Glx | L-glutamic acid or |
| | | L-glutamine |
| W | Trp | L-tryptophan |
| R | Arg | L-arginine |
| D | Asp | L-aspartic acid |
| N | Asn | L-asparagine |
| B | Asx | L-aspartic acid or |
| | | L-asparagine |
| C | Cys | L-cysteine |

STABILITY As used in reference to supramolecular assemblages, including VLPs, stability refers to structural and biochemical integrity of the particles. Stability can be directly measured, for example, by analytical size-exclusion chromatography followed and analysis of the resulting elutant profile, electron microscopy, native and non-native gel electrophoretic techniques coupled with various detection methodologies including staining and immunodetection, rate-zonal centrifugation, nuclease protection assays for supramolecular assemblages (VLPs) that encapsidate nucleic acids, light scattering, or other biophysical and biochemical techniques as are known in the art.

An inventive supramolecular assemblage (VLP) is stabilized when the amount of unassembled chimeric capsid polypeptide in a supramolecular assemblage (VLP) preparation (as a fraction of total chimeric polypeptide) is reduced by at least about 25 percent relative to the amount of unassembled capsid polypeptide lacking an attached self-binding peptide in an otherwise identical supramolecular assemblage (VLP) preparation (as a fraction of total capsid polypeptide). Using analytical size exchange chromatography, for example, unassembled capsid polypeptide is measured as the area under the curve of slowly eluting capsid polypeptide divided by the sum of areas under the curve of both fast and slowly eluting polypeptide. A preparation is considered to represent a stabilized supramolecular assemblage (VLP) when the amount of unassembled chimeric capsid polypeptide (with an attached self-binding peptide) as a fraction of the total in a first supramolecular assemblage (VLP) preparation is at least about 25 percent less than the unassembled capsid polypeptide in a second supramolecular assemblage (VLP) preparation that is identical to the first except that the capsid polypeptide lacks an attached self-binding peptide. The same quantification of unassembled capsid polypeptide as a proportion of total in a supramolecular assemblage (VLP) preparation is available for most analytical techniques.

ANALOGUE As used herein, a peptide or polypeptide analogue is a peptide or polypeptide bearing at least about 50 percent identity to a referent peptide or polypeptide. The analogue is also referred to a being analogous to the referent peptide or polypeptide. A nucleic acid sequence analogue encodes a peptide or polypeptide that is at least about 50 percent identical to a peptide or polypeptide encoded by a referent nucleic acid sequence. As such, an analogue nucleic acid need not have 50 percent or more sequence identity with the referent nucleic acid in view of the well known redundancy of the genetic code, nor need it have any similarity beyond a coding region.

### DETAILED DESCRIPTION OF THE INVENTION

This invention provides a supramolecular assemblage that is comprised of a chimeric polypeptide having two portions, a first portion and a covalently linked (attached) second potion. The first portion self-assembles into particles whether the second portion is present or not. The second portion comprises a self-binding peptide sequence having a length of about 15 to about 80 amino acid residues, and preferably about 15 to about 35 residues. That peptide a) when present as an N-acetylated peptide self-assembles to form parallel multimers in aqueous PBS solution at pH 7.0 and a concentration of about 10 millimoles/liter, or b) self assembles when present in solution at a concentration of at least ten micromoles/liter in the presence of a five-fold molar excess of a predetermined multivalent metal ion, or c) is the self-assembling extracellular domain of the influenza A M2 polypeptide, with the proviso that the M2 peptide second portion is not present at the N-terminus of HBc as a first polypeptide portion. The chimeric polypeptide forms organized, repetitive, supramolecular structures that are more stable than are structures formed from said first portion alone.

That is, a contemplated chimeric polypeptide forms a particle that is more stable (as defined elsewhere) than is a particle formed from a polypeptide that is otherwise identical in sequence but lacks that covalently attached self-binding, second peptide sequence; i.e., a polypeptide having the sequence of only the first portion. In some embodiments, the chimeric polypeptide is substantially free of nucleic acid binding properties, whereas in other embodiments, the chimeric polypeptide binds to nucleic acids. A chimeric polypeptide or supramolecular assemblage (VLP) can include one or more attached heterologous epitopes (haptens or fused polypeptide sequences).

For ease in writing, the acronym VLP is used hereinafter for supramolecular assemblage, except where the context of usage indicates that one or more specific virally-related VLPs is intended.

The disclosed, stabilized VLPs and stabilized VLPs with attached heterologous epitopes are useful as vaccines or immunogens for one or both of the production of antibodies or for activation of T cells. The invention also provides for a stabilized, ordered, repetitive epitope array through the attachment of a heterologous epitope to a capsid polypeptide with a self-binding peptide component.

In a preferred embodiment, the present invention provides a chimeric polypeptide composition comprising two portions, a first portion that self-assembles into an organized, repetitive polypeptide, supramolecular structure having at least about 9 polypeptide subunits and a second self-binding peptide portion, where these portions are attached (linked) by at least one covalent bond. In some preferred embodiments of the invention, the two portions are attached by a peptide bond. In some of those embodiments, the peptide bond attachment of the first portion of the chimeric polyprotein to the second portion arises through an in-frame fusion of nucleotides encoding the first portion to nucleotides encoding the second portion followed by expression of the resulting polynucleotide in a host cell to form a fusion polypeptide on expression in a host cell.

In other embodiments, the two portions are attached via bifunctional linking agents acting on linker residues such as lysine and cysteine. Suitable bifunctional reagents include SMCC, MBS and N-succinimidyl-3-(2-pyridyldithio)proprionate (SPDP), and the like as are noted in the 2001-2002 Catalog of Pierce Biotechnology, Inc. Rockford, Ill. The bifunctional reagents generally include a group that forms a bond through an amine group and a group capable of forming a disulphide or thioether link.

A sulfhydryl group can be provided as an N-terminal and/or C-terminal Cys residue or by reaction of amino functions with 2-iminothiolane or the N-hydroxy-succinimide ester of 3-(2-dithiopyridyl)propionate or S-acetylthioglycolic acid. After reaction with S-acetylthioglycolic acid N-hydroxysuccinimide ester (SATA) deacetylation of the SATA with, for example, hydroxylamine provides a free —SH group. Where a free amino group is required, a lysine can be provided by genetic engineering, protein ligation or other methods. Amino acids other than α-amino acids such as β-alanine, γ-aminobutyric acid and ε-aminocaproic acid can also be used as linkers.

In one preferred embodiment, the second self-binding peptide portion is attached to the N-terminus or the C-terminus of the first portion of the chimeric polypeptide. In a further preferred embodiment, the self-binding peptide is attached to the N-terminus or C-terminus of the first portion by genetic fusion of appropriate nucleic acid sequences and co-expression so that the chimeric polypeptide is expressed as a fusion of the first and second portions and the first and second portions are covalently linked by a peptide bond. In another preferred embodiment, the first portion is provided a linker residue at or near the N- or C-terminus of the first portion adapted to attaching the self-binding peptide.

It is to be noted that the “first portion” and the “second portion” are so named for the sake of convenience only. As a consequence, a “second portion” self-binding peptide can precede, be N-terminal to, the “first portion” polypeptide in a contemplated chimeric polypeptide.

In another embodiment, the chimer polypeptide first portion comprises a viral capsid polypeptide in whole or truncated form. In some embodiments, exclusive of a heterologous epitope or linker for a heterologous epitope, the amino acid residue sequence of the first portion that is present is greater than about 50 percent, preferably greater than about 75 percent, more preferably greater than about 90 percent and most preferably greater than about 95 percent identical to a virus capsid polypeptide. Thus, where a first portion comprises a viral protein sequence such as residues 1-149 of the hepatitis B virus core (HBc) protein, the sequence that is present in the first portion is 100 percent identical to the viral sequence. However, residues 1-149 represent a truncated version of HBc because residues 150-183 are absent. A first portion that self-assembles into an organized, repetitive, supramolecular structure that contains at least about 9 polypeptide subunits and exhibits an above-noted identity percentage to a viral capsid polypeptide sequence allele is defined herein as a viral capsid analogue (or analog) polypeptide sequence.

In further preferred embodiments of this invention, the first portion of a chimeric polypeptide is a virus capsid polypeptide or an analogue of a capsid polypeptide of a virus that can be an animal-infecting virus, a plant-infecting virus, a fungus-infecting virus or a bacteria-infecting virus. In preferred embodiments, the virus capsid polypeptide that forms the VLP is a capsid polypeptide from a member of the virus families Picornaviridae, Caliciviridae, Togaviridae, Flaviviridae, Rhabdoviridae, Paramyxoviridae, Orthomyxoviridae, Reoviridae, Retroviridae, Polyomaviridae, Papillomaviridae, Adenoviridae, Parpovaviridae, Hepadnaviridae, Nodaviridae, Tetraviridae, Tombusviridae, Comoviridae, Bromoviridea, Potyviridea, Inoviridae, Leviviridae, Microviridae, Pseudoviridae, and Totiviridae, or an analogue thereof. In other preferred embodiments the virus capsid polypeptide that forms the VLP is that of a member of the genus Tobamovirus, Potexvirus, or Tymovirus, or an analogue of a capsid of such a virus.

In other preferred embodiments, the first portion of the chimeric polypeptide comprises the self-assembling, icosahedron-forming polypeptide of an icosahedral enzyme complex. In some preferred aspects, the first portion comprises the E2 subunit of the pyruvate dehydrogenase enzyme complex. In other preferred aspects, the first portion comprises the lumazine synthase polypeptide. As with the virus capsid amino acid residue sequences above, in some aspects, the first portion comprises an analogue of the icosahedral enzyme complex polypeptide and exclusive of a heterologous epitope or linker for a heterologous epitope, the first portion has an amino acid sequence that is greater than about 50 percent, preferably greater than about 75 percent, more preferably greater than about 90 percent and most preferably greater than about 95 percent identical to an icosahedral enzyme complex subunit polypeptide.

In some preferred embodiments, the first portion of the chimeric polypeptide is substantially free of nucleic acid binding properties. Here, in some embodiments, the first portion of the chimeric polypeptide is substantially free of nucleic acid binding and includes a first attachment site to which an epitope bearing a second attachment site can be attached through one or more covalent or non-covalent bonds. Other embodiments can contain chimeric polypeptides that bind nucleic acid and also include a first attachment site for an epitope. The bound nucleic acids are typically oligomeric and/or polymeric DNA and RNA species originally present in the cells of the organism used to express the protein.

The binding of nucleic acid and the substantial freedom from nucleic acid binding can be readily determined by a comparison of the absorbance of the chimeric particles in aqueous solution measured at both 280 and 260 nm; i.e., a 280/260 absorbance ratio. Nucleic acids that are bound exhibit an absorbance at 260 nm and relatively less absorbance at 280 nm, whereas a protein free of bound nucleic acid absorbs relatively less at 260 nm and has a greater absorbance at 280 nm.

Thus, illustrative recombinantly expressed chimeric hepatitis B core or capsid (HBc) particles that bind nucleic acids contain some or all of the arginine-rich sequences at residue positions 150-183 (or 150-185) exhibit a ratio of absorbance at 280 nm to absorbance at 260 nm (280:260 absorbance ratio) of about 0.8, whereas similar particles free of the arginine-rich nucleic acid binding region of naturally occurring HBc such as those that contain fewer than four arginine or lysine residues or mixtures thereof adjacent to each other, or those HBc particles assembled from monomeric polypeptide chimer molecules having a native or chimeric sequence that ends at about HBc residue position 140 to position 149, exhibit a 280:260 absorbance ratio of about 1.2 to about 1.6. The HBc polypeptide and particles that self-assemble from HBc are frequently used illustratively herein.

Chimeric particles of the present invention that are substantially free of nucleic acid binding and exhibit a 280:260 absorbance ratio of about 1.2 to about 1.7, and more typically, about 1.4 to about 1.6.

One preferred embodiment contemplates a stabilized VLP comprising a polypeptide with a first portion that is a virus capsid polypeptide or an analogue thereof, as described above, attached to a second portion comprising a heterologous self-binding peptide. In another preferred embodiment, the invention contemplates a stabilized VLP comprising a chimeric polypeptide that comprises a first portion that is a virus capsid polypeptide or an analogue thereof that includes a linker residue to which an epitope can be attached and a second portion that comprises a heterologous self-binding peptide. In a further preferred embodiment, the invention is a stabilized VLP comprising a chimeric polypeptide whose first portion is a virus capsid polypeptide or analogue thereof that includes an attached heterologous epitope and a second portion that comprises a heterologous self-binding peptide.

A further preferred embodiment contemplates a stabilized VLP having a first portion comprising the HBc polypeptide truncated at about amino acid 139 to about position 165, and preferably to about 156 and more preferably to about 149 (as counted from the N-terminus) with the second portion comprising a self-binding peptide as discussed before and hereinafter in greater detail.

In another preferred embodiment of the invention, the VLP first portion comprises or is an analogue of the capsid polypeptide from a virus in the group consisting of the MS2 bacteriophage, JC virus, the Ty1 retrotransposon, HIV, flock house virus, Nudaurelia capensis omega virus, brome mosaic virus, Tomato bushy stunt virus, turnip crinkle virus, HPV 16, Norwalk virus, B19 virus, potato virus X, sindbis virus, Physalis mottle virus, fd bacteriophage, the phiX174 bacteriophage, rotavirus, tobacco mosaic virus, the cowpea mosaic virus Reovirus, hepatitis C virus, measles virus, influenza A virus, adenovirus, the L-A virus and polio virus.

An illustrative and preferred recombinant chimer that forms a VLP comprises a hepatitis B core (HBc) protein molecule up to about 570 amino acid residues in length that (a) contains a sequence of at least about 135 of the N-terminal 150 amino acid residues of the HBc molecule that include a peptide-bonded heterologous epitope or a heterologous linker residue for a conjugated epitope present in the HBc immunodominant loop, or a sequence of at least about 135 residues of the N-terminal 150 HBc amino acid residues, (b) contains a C-terminal self-binding sequence of amino acid residues having a length of about 15 to about 35 residues, and (c) contains a sequence of at least 5 amino acid residues from HBc position 135 to the HBc C-terminus. The self-binding sequence a) when present as an N-acetylated peptide self-assembles to form parallel multimers in aqueous PBS solution at pH 7.0 and a concentration of about 10 millimoles/liter, or b) self assembles when present in solution at a concentration of at least ten micromoles/liter in the presence of a five-fold molar excess of a predetermined multivalent metal ion, or c) is the self-assembling extracellular domain of the influenza A M2 polypeptide. The chimer molecules contain no more than 10 percent conservatively substituted amino acid residues in the HBc sequence. The particles are more stable than are particles formed from an otherwise identical HBc chimer that lacks the C-terminal self-binding sequence. The particles preferably self-assemble into particles that are substantially free of binding to nucleic acids.

A chimer in which an M2 polypeptide second portion containing zero, one or two cysteine residues is present at the N-terminus of an HBc first portion polypeptide is expressly excluded from this invention. On the other hand, a chimer containing an HBc first portion C-terminally bonded M2 second portion containing zero, one or two cysteines, is particularly preferred.

In other embodiments, the first portion of the chimeric polypeptide is or is an analogue of the self-assembling E2 polypeptide of the pyruvate dehydrogenase complex or is or is an analogue of the self-assembling lumazine synthase polypeptide.

Self-Binding Peptides

The second, self-binding peptide portion of a contemplated chimeric polypeptide is a sequence about 15 to about 80, and more preferably about 15 to about 35 amino acids in length. The second, self-binding peptide portion of the chimeric polypeptide self-assembles into parallel multimers in aqueous PBS solution at pH 7.0 at a concentration of about 10 millimolar when present as an N-acetylated peptide. In another preferred embodiment, the self-binding peptide forms parallel multimers when present as an N-acetylated peptide and provided an appropriate metal ion. In a further preferred embodiment, the self-binding peptide is the N-terminal about 24 amino acids of the influenza A M2 protein.

In another preferred embodiment, the second self-binding peptide portion of the chimeric polypeptide comprises the GCN4-p1 [O'Shea et al. (1989) Science 243:538-542] leucine zipper peptide. In another embodiment, the self-binding peptide portion comprises the engineered leucine zipper peptide GCN4-II, GCN4-IL, GCN4-LI, GCN4-LV, GCN4-VL, or GCN4-LL. [Harbury et al. (1993) Science 262:1401-1407] In another preferred embodiment the self-binding peptide is the pentamerization peptide of the cartilage oligomeric matrix protein (COMP). [Ozbek. (2002) EMBO J. 21 (22), 5960-5968] In another preferred embodiment the self-binding peptide is the right-handed tetrameric self-binding sequence of tetrabrachion. [Stetefeld (2000) Nat. Struc. Biol. 7:772-776.]

In another aspect, the self-binding peptide is derived from a human protein sequence. Thus, illustratively, the self-binding peptide is the leucine zipper from a human protein sequence chosen from the group Mad, Max, c-Myc, N-Myc, L-Myc, AP4, and USF. [Canne et al. (1995) J. Am. Chem. Soc. 117:2998; and Blackwood et al. (1991) Science 251:1211-1217]

Exemplary amino acid residue sequences of illustrative second portion self-binding peptides are listed in Table I, hereinbelow, along with their respective peptide names, sequence ID numbers and citations.

TABLE I
Self-Binding Peptides

| Peptide Name | Sequence | SEQ ID NO | Citation |
|---|---|---|---|
| GCN4-p1 | Ac-RMKQLEDKVEELLSKNYHL- | 10 | 1 |
| | ENEVARLKKLVGER | | |
| Max | YQYMRKNHTHQQDIDDLKRQNALL- | 11 | 3 |
| | EQQVRALEKAR | | |
| SMax | RKNDTLQQDIDDLKRQNALL- | 12 | 2 |
| | EQQVRALEKAR | | |
| c-Myc | YLSVQAEEQKLISEEDLL- | 13 | 3 |
| | RKRREQLKHKLEQL | | |
| N-Myc | YHSVQAEEHQLLLEKEKL- | 14 | 3 |
| | QARQQQLLKKIEHA | | |
| L-Myc | LQALVGAEKRMATEKRQL- | 15 | 3 |
| | RCRQQQLQKRIAYL | | |
| AP-4 | IFSLEQEKTRLLQQNTQL- | 16 | 3 |
| | KRFIQEL | | |
| USF | IQELRQSNHQLQLDNDVL- | 17 | 3 |
| | RQQVEDLKNKNLLL- | | |
| | RLSEELQGLD | | |
| GCN4-IL | Ac-RIKQLEDKIEELLSKIYHL- | 18 | 4 |
| | ENEIARLKKLVGER | | |
| GCN4-II | Ac-RIKQIEDKIEEILSKIYHI- | 19 | 4 |
| | ENEIARIKKLVGER | | |
| GCN4-LI | Ac-RLKQIEDKLEEILSKLYHI- | 20 | 4 |
| | ENELARIKKLVGER | | |
| GCN4-VI | Ac-RVKQIEDKVEEILSKVYHI- | 21 | 4 |
| | ENEVARIKKLVGER | | |
| GCN4-LV | Ac-RLKQVEDKLEEVLSKLYHV- | 22 | 4 |
| | ENELARVKKLVGER | | |
| GCN4-VL | Ac-RVKQLEDKVEELLSKVYHL- | 23 | 4 |
| | ENEVARLKKLVGER | | |
| GCN4-LL | Ac-RLKQLEDKLEELLSKLYHL- | 24 | 4 |
| | ENELARLKKLVGER | | |
| Metal Binders | Ac-GLAQKLLEALQKALA-CONH₂ | 25 | 5 |
| | Ac-GELAEQKLEQALQKLA-CONH₂ | 26 | 5 |
| RHCC | GSIINETADDIVYRLTVII- | 27 | 6 |
| | DDRYESLKNLITLRAD- | | |
| | RLEMIINDNVSTILASI | | |
| COMP (m) | MDLAPQMLRELQETNAAL- | 28 | 7 |
| | QDVRELLRQQVKEI- | | |
| | TFLKNTVMECDAC | | |
| COMP (hu) | DLGPQMLRELQETNAAL- | 29 | 8 |
| | QDVRDWLRQQVREI- | | |
| | TFLKMTVMECDAC | | |
| M2 (2-24/ | ISLLTEVETPIRNEWG- | 30 | 9 |
| C17S, C19S) | SRSNDSSDEL | | |

1. O′Shea et al. (1989) Science 243: 538-542.
2. Canne et al. (1995) J. Am. Chem. Soc. 117: 2998.
3. Blackwood et al. (1991) Science 251: 1211-1217.
4. Harbury et al. (1993) Science 262: 1401-1407.
5. Ghadiri et al., (1992) J. Am. Chem. Soc. 114: 4000-4002.
6) Stetefeld (2000) Nat. Struc. Biol. 7: 772-776.
7) Ozbek. (2002) EMBO J. 21 (22), 5960-5968.
8) Newton (1994) Genomics 24: 435-439.
9) This disclosure.

In some embodiments of this invention, the first portion includes a first attachment site for a heterologous epitope having at least one second attachment site. The first and second attachment sites associate by way of at least one covalent or non-covalent bond. Thus, the epitope and the chimeric polypeptide are brought together through the association of the first and the second attachment site. For example, a biotin binding sequence (as noted hereinafter) can be co-expressed as a fusion polypeptide with a first portion. The heterologous epitope linked to biotin can be admixed with the stabilized VLP formed from the fusion polypeptide so that the biotin-linked epitope binds to the biotin binding sequence of the VLP.

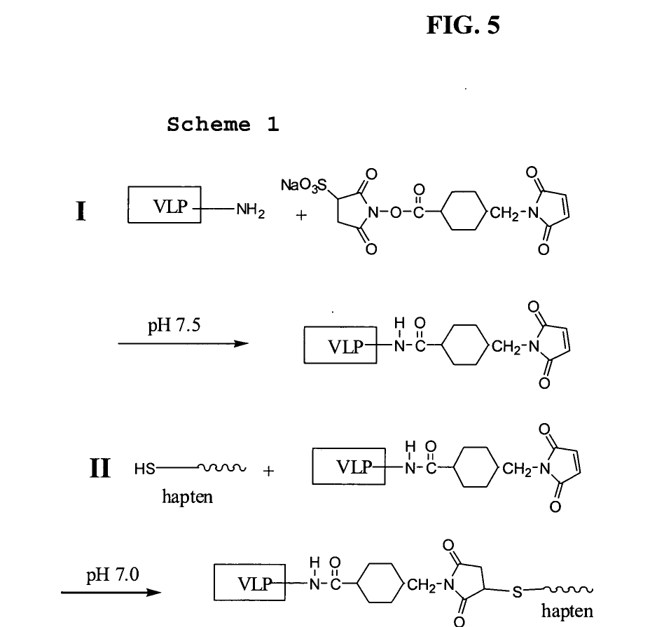

The first attachment site can be a linker residue adapted to react with a bifunctional linking agent as discussed before, and the second attachment site can also be adapted to react with the same bifunctional linking agent. The adaptation of the second attachment site can comprise the same or a different linker residue such as lysine or cysteine residue, a free amino group or a free sulfhydral group of a hapten or other immunogen. The presence of a first attachment site as a portion of the first portion is shown in Scheme 1 of FIG. 5, along with a second attachment site present on a heterologous epitope. See also, U.S. Pat. No. 6,231,864.

In another embodiment of this invention the first portion includes a first attachment site for a heterologous epitope adapted for attachment by non-covalent interactions to a second attachment site on an epitope. Examples include a chimeric polypeptide with an engineered site such as a lysine or a cysteine residue as a first attachment site that can react with biotin or a compound such as 3-(maleimido-propionyl)biotin to form a biotinylated first polypeptide portion and an epitope having as a second attachment site a linked avidin, strepavidin or neutral, low molecular weight avidin analogue such as that disclosed in Marttila et al., (February 2000) FEBS Lett. 467(1):31-36 and referred to therein as NeutraLite™ avidin. In other embodiments, the attachments are through ligand/receptor interactions as are known in the art.

In some preferred embodiments of the invention, the epitope is an amino acid residue sequence that is heterologous to the VLP. An illustrative sequence that is normally present in a human or animal pathogen or a cancer protein is present in a contemplated chimeric polypeptide of a stabilized VLP in a sequence that is shorter in length than the naturally occurring protein in which the epitope is present. Such shortened peptide sequences and other small molecules that are not immunogenic in themselves but become immunogenic once attached to a carrier protein such as a contemplated stabilized VLP are referred to as haptens. An exemplary heterologous sequence can contain up to about 245 amino acid residues that are heterologous (foreign) to the VLP.

In certain embodiments, the epitope comprises an amino acid residue sequence present in a pathogen chosen from the group consisting of Ebola virus, HIV-1, HIV-2, foot-and-mouth disease virus (FMDV), hepatitis B virus, hepatitis C virus, influenza A virus, the human papilloma viruses, West Nile virus, yellow fever virus, Streptococcus pneumonia, Cryptosporidium parvum, Vibrio cholerae, Yersinia pestis, Haemophilus influenzae, Moraxella catarrhalis, Porphyromonas gingivalis, Trypanosoma cruzi, Plasmodium falciparum, Plasmodium vivax, Plasmodium berghi, Plasmodium yoelli, Streptococcus sobrinus, Shigella flexneri, Bacillus anthracis, Entamoeba histolytica, Schistosoma japonicum, Schistosoma mansoni, and Neisseria meningitidis. In some other embodiments, the epitope is a peptide derived from a cancer antigen.

In other embodiments, the epitope is an oligosaccharide, lipopolysaccharide, lipoprotein, glycoprotein or proteoglycan hapten. Illustrative B and T cell peptidal epitopes are listed in Tables A and B, hereinbelow, along with the common name given to the gene from which the sequence is obtained, the literature or patent citation for published epitopes, and SEQ ID NO. These peptidal epitopes can be fused into the sequence of the first portion polypeptide or can be present as separately bonded hapten epitopes, as discussed before. Saccharidal haptens are discussed hereinafter.

TABLE A
B Cell Epitopes

| Organism | Gene | Sequence | Citation* | SEQ ID NO |
|---|---|---|---|---|
| Streptococcus | PspA1 | KLEELSDKIDELDAE | 1 | 31 |
| pneumoniae | PsP2 | QKKYDEDQKKTEE- | 1 | 32 |
| | | KAALEKAASEEM- | | |
| | | DKAVAAVQQA | | |
| Cryptosporidium | P23 | QDKPADAPAAEAPA- | 2 | 33 |
| parvum | | AEPAAQQDKPADA | | |
| HIV | GP120 | RKRIHIGPGR- | 3 | 34 |
| | | AFYITKN | | |
| | | SNCTRPNNNTR- | 49 | 35 |
| | | KSIRIQRGPG- | | |
| | | RAFVTIGKIG- | | |
| | | NMRQAHCNISG | | |
| | | SNCTRPNNNTR- | 49 | 36 |
| | | KRIRIQRGPG- | | |
| | | RAFVTIGKIG- | | |
| | | NMRQAHCNISG | | |
| | | SNCTRPNYNKR- | 49 | 37 |
| | | KRIHIGPG- | | |
| | | RAFYTTKNIIG- | | |
| | | TIRQAHCNISG | | |
| | | SNCTRPGNNTR- | 49 | 38 |
| | | RGIHFGPG- | | |
| | | QALYTTGIVD- | | |
| | | IRRAYCTING | | |
| | | SNCTRPNNNTR- | 49 | 39 |
| | | KSITKGPG- | | |
| | | RVIYATGQIIGD- | | |
| | | IRAHCNLSGS | | |
| | | STCARPYQNTR- | 49 | 40 |
| | | QRTPIGLG- | | |
| | | QSLYTTRSRS- | | |
| | | IIGQAHCNISG | | |
| | | SNCTRPGNNTR- | 49 | 41 |
| | | RGIHFGPG- | | |
| | | QALYTTGIVD- | | |
| | | EIRRAYCNISG | | |
| | | SNCTRPNNTR- | 49 | 42 |
| | | KSITKQRGPG- | | |
| | | RVLYATGQIIGD- | | |
| | | IRKAHCNSIG | | |
| | | STCARPYQNTR- | 49 | 43 |
| | | QRTPIGLG- | | |
| | | QSLYTTRGRTKI- | | |
| | | IGQAHCNISG | | |
| Foot-and-mouth | VP1 | YNGECRYNRNA- | 4 | 44 |
| disease virus | | VPNLRGDLQVL- | | |
| | | AQKVARTLP | | |
| Influenza Virus | HA | YRNLLWLTEK | 8 | 45 |
| A8/PR8 | | | | |
| Type A | M2 | SLLTEVETPIR- | 29 | 46 |
| (A8/PR8/34) | | NEWGCRCNGSSD | | |
| | | SLLTEVETPIR- | 29 | 47 |
| | | NEWGCRCNDSSD | | |
| | | SLLTEVETPIR- | | 48 |
| | | NEWGARANDSSD | | |
| | | EQQSAVDADDS- | 35 | 49 |
| | | HFVSIELE | | |
| | | SLLTEVETPIR- | | 50 |
| | | NEWGSRSNDSSD | | |
| | | SLLTEVETPIR- | | 51 |
| | | NEWGSRCNDSSD | | |
| | | SLLTEVETPIR- | | 52 |
| | | NEWGCRSNDSSD | | |
| | | SLLTEVETPIR- | | 53 |
| | | NEWGCRANDSSD | | |
| | | SLLTEVETPIR- | | 54 |
| | | NEWGARCNDSSD | | |
| | | MSLLTEVETPIR- | | 55 |
| | | NEWGCRCNDSSD | | |
| | | MSLLTEVETPIR- | | 56 |
| | | NEWGSRSNDSSD | | |
| | | MGISLLTEVETPIR- | | 57 |
| | | NEWGCRCNDSSD- | | |
| | | ELLGWLWGI | | |
| | | MSLLTEVETPIR- | | 58 |
| | | NEWGARANDSSD | | |
| | | MSLLTEVETPIR- | | 59 |
| | | NEWGCRANDSSD | | |
| | | MSLLTEVETPIR- | | 60 |
| | | NEWGARCNDSSD | | |
| | | MSLLTEVETPIR- | | 61 |
| | | NEWGCRSNTDSSD | | |
| | | MSLLTEVETPIR- | | 62 |
| | | NEWGSRCNDSSD | | |
| | | SLLTEVETPIRNEWGS- | | 63 |
| | | RSNDSSDSLLTEVET- | | |
| | | PIRNEWGSRSNDSSD | | |
| | | SLLTEVETPIRNEWGS- | | 64 |
| | | RSNDSSDSLLTEVETPIR- | | |
| | | NEWGSRSNDSSDSLLTEV- | | |
| | | ETPIRNEWGSRSNDSSD | | |
| | | SLLTEVETPIRNEWGARAND- | | 65 |
| | | SSDSLLTEVETPIRNEWG- | | |
| | | ARANDSSD | | |
| | | SLLTEVETPIRNEWGA- | | 66 |
| | | RANDSSDSLLTEVETPIR- | | |
| | | NEWGARANDSSDSLLTEV- | | |
| | | ETPIRNEWGARANDSSD | | |
| | | EVETPIRNEWGSRCNDSSD | | 67 |
| | | EVETPIRNEWGSRCNDSSDEV- | | 68 |
| | | ETPIRNEWGSRCNDSSD | | |
| | | EVETPIRNEWGSRCNDSSDEV- | | 69 |
| | | ETPIRNEWGSRCNDSSDE- | | |
| | | VETPIRNEWGSRCNDSSD | | |
| | | X₁X₂X₃X₄X₅X₆X₇X₈TPIRNE- | | 70 |
| | | X₁₅X₁₆X₁₇X₁₈X₁₉X₂₀- | | |
| | | X₂₁X₂₂X₂₃X₂₄ | | |
| | | X₁X₂X₃X₄X₅X₆X₇X₈TPIRNE- | | |
| | | X₁₅X₁₆X₁₇X₁₈X₁₉X₂₀X₂₁X₂₂- | | |
| | | X₂₃X₂₄X₁X₂X₃X₄X₅X₆X₇X₈- | | |
| | | TPIRNEX₁₅X₁₆X₁₇X₁₈X₁₉- | | |
| | | X₂₀X₂₁X₂₂X₂₃X₂₄ | | 71 |
| | | X₁X₂X₃X₄X₅X₆X₇X₈TPIRNE- | | 72 |
| | | X₁₅X₁₆X₁₇X₁₈X₁₉X₂₀X₂₁- | | |
| | | X₂₂X₂₃X₂₄X₁X₂X₃X₄X₅X₆X₇₋ | | |
| | | X₈TPIRNEX₁₅X₁₆X₁₇X₁₈X₁₉- | | |
| | | X₂₀X₂₁X₂₂X₂₃X₂₄X₁X₂X₃X₄- | | |
| | | X₅X₆X₇X₈TPIRNEX₁₅X₁₆X₁₇- | | |
| | | X₁₈X₁₉X₂₀X₂₁X₂₂X₂₃X₂₄ | | |
| | | X₁X₂X₃X₄X₅X₆X₇X₈TX₁₀X₁₁R- | | 73 |
| | | X₁₃X₁₄X₁₅X₁₆X₁₇X₁₈X₁₉- | | |
| | | X₂₀X₂₁X₂₃X₂₄ | | |
| | | X₁X₂X₃X₄X₅X₆X₇X₈TX₁₀X₁₁R- | | 74 |
| | | X₁₃X₁₄X₁₅X₁₆X₁₇X₁₈X₁₉X₂₀- | | |
| | | X₂₁X₂₂X₂₃X₂₄X₁X₂X₃X₄X₅X₆- | | |
| | | X₇X₈TX₁₀X₁₁RX₁₃X₁₄X₁₅X₁₆- | | |
| | | X₁₇X₁₈X₁₉X₂₀X₂₁X₂₂X₂₃X₂₄ | | |
| | | X₁X₂X₃X₄X₅X₆X₇X₈TX₁₀X₁₁R- | | 75 |
| | | X₁₃X₁₄X₁₅X₁₆X₁₇X₁₈X₁₉X₂₀- | | |
| | | X₂₁X₂₂X₂₃X₂₄X₁X₂X₃X₄X₅X₆- | | |
| | | X₇X₈TX₁₀X₁₁RX₁₃X₁₄X₁₅X₁₆- | | |
| | | X₁₇X₁₈X₁₉X₂₀X₂₁X₂₂X₂₃X₂₄- | | |
| | | X₁X₂X₃X₄X₅X₆X₇X₈TX₁₀X₁₁R- | | |
| | | X₁₃X₁₄X₁₅X₁₆-X₁₇X₁₈X₁₉- | | |
| | | X₂₀X₂₁X₂₂X₂₃X₂₄ | | |
| Type B | NB | NNATFNYTNVNPISHIR | | 76 |
| | M2 | MLEPFQ | 47 | 77 |
| | | MLEPLQ | | 78 |
| Yersinia | V Ag | DILKVIVDSMNHH- | 9 | 79 |
| pestis | | GDARSKLREELAE- | | |
| | | LTAELKIYSVIQA- | | |
| | | EINKHLSSSGTIN- | | |
| | | IHDKSINLMDKNL- | | |
| | | YGYTDEEIFKASA- | | |
| | | EYKILEKMPQTTI- | | |
| | | QVDGSEKKIVSIK- | | |
| | | DFLGSENKRTGAL- | | |
| | | GNLKNSYSYNKDN- | | |
| | | NELSHFATTCSD | | |
| Haemophilus | pBOMP | CSSSNNDAA- | 10 | 80 |
| influenza | | GNGAAQFGGY | | |
| | | NKLGTVSYGEE | | 81 |
| | | NDEAAYSKN- | | 82 |
| | | RRAVLAY | | |
| Moraxella | copB | LDIEKDKKK- | 11 | 83 |
| catarrhalis | | RTDEQLQAE- | | |
| | | LDDKYAGKGY | | |
| | | LDIEKNKKK- | | 84 |
| | | RTEAELQAE- | | |
| | | LDDKYAGKGY | | |
| | | IDIEKKGKI- | | 85 |
| | | RTEAELLAE- | | |
| | | LNKDYPGQGY | | |
| Porphyromonas | HA | GVSPKVCKDVTV- | 12 | 86 |
| gingivalis | | EGSNEFAPVQNLT | | |
| | | RIQSTWRQKTV- | | 87 |
| | | DLPAGTKYV | | |
| Trypanosoma | | KAAIAPAKAAA- | 14 | 88 |
| cruzi | | APAKAATAPA | | |
| Plasmodium | CS | (NANP)₄ | 24 | 89 |
| falciparum | | NANPNVDP- | | 90 |
| | | (NANP)₃NVDP | | |
| | | NANPNVDP- | | 91 |
| | | (NANP)₃ | | |
| | | (NANP)₃NVDPNANP | | 92 |
| | | NANPNVDP- | | 93 |
| | | (NANP)₃NVDPNANP | | |
| | | NPNVDP (NANP)₃NV | | 94 |
| | | NPNVDP- | | 95 |
| | | (NANP)₃NVDP | | |
| | | NPNVDP (NANP)₃- | | 96 |
| | | NVDPNA | | |
| | | NVDP (NANP)₃NV | | 97 |
| | | NVDP (NANP)₃NVDP | | 98 |
| | | NVDP (NANP)₃- | | 99 |
| | | NVDPNA | | |
| | | DP (NANP)₃NV | | 100 |
| | | DP (NANP)₃NVDP | | 101 |
| | | DP (NANP)₃- | | 102 |
| | | NVDPNA | | |
| vivax | CS | GDRADGQPAG- | 20 | 103 |
| | | DRADGQPAG | | |
| | | RADDRAAGQP- | | 104 |
| | | AGDGQPAG | | |
| | | ANGAGNQPG- | | 105 |
| | | ANGAGDQPG | | |
| | | ANGADNQPG- | 27 | 106 |
| | | ANGADDQPG | | |
| | | ANGAGNQPG- | | 107 |
| | | ANGADNQPG | | |
| | | ANGAGNQPG- | | 108 |
| | | ANGADDQPG | | |
| | | APGANQEGGAA- | 28 | 109 |
| | | APGANQEGGAA | | |
| | | ANGAGNQPGAN- | | 110 |
| | | GAGDQPGANGA- | | |
| | | DNQPGANGADD- | | |
| | | QPG | | |
| berghi | CS | DPPPPNPN- | 2 | 111 |
| | | DPPPPNPN | | |
| yoelli | CS | (QGPGAP)₄ | | 112 |
| Streptococcus | AgI/II | KPRPIYEA- | 16 | 113 |
| sobrinus | | KLAQNQK | | |
| | | AKADYEAK- | | 114 |
| | | LAQYEKDL | | |
| Shigella | Invasin | KDRTLIEQK | 18 | 115 |
| flexneri | | | | |
| Respiratory syncitia | G | CSICSNNPT- | 19 | 116 |
| virus (RSV) | | CWAICK | | |
| Entamoeba | lectin | VECASTVCQNDN- | 21 | 117 |
| histolytica | | SCPIIADVEKCNQ | | |
| Schistosoma | para | DLQSEISLSLE- | 22 | 118 |
| japonicum | | NGELIRRAKSA- | | |
| | | ESLASELQRRVD | | |
| Schistosoma | para | DLQSEISLSLE- | 22 | 119 |
| mansoni | | NSELIRRAKAA- | | |
| | | ESLASDLQRRVD | | |
| Bovine Inhibin | α_{c} subunit | STPPLPWPW- | 30 | 120 |
| | | SPAALRLLQ- | | |
| | | RPPEEPAA | | |
| Ebola Virus | membrane-anchored glycoprotein | ATQVEQHHRR- | 31 | 121 |
| | | TDNDSTA | | |
| | | HNTPVYKLD- | 31 | 122 |
| | | ISEATQVE | | |
| | | GKLGLITNTI- | 31 | 123 |
| | | AGVAVLI | | |
| Escherichia coli | ST | CCELCCYPACAGCN | 33 | 124 |
| | | NTFYCCELCC- | | |
| | | YPACAGCN | 33 | 125 |
| | | SSNYCCELCC- | 33 | 126 |
| | | YPACAGCN | | |
| Alzheimer's disease | β-Amyloid | DAEFRHDSGYE- | 34 | 127 |
| | | VHHQKLVFFAE- | | |
| | | DVGSNKGAIIG- | | |
| | | LMVGGVVIA | | |
| | | DAEFRHDSGYE- | | 128 |
| | | VHHQKL | | |
| | | EDVGSNKGAII | | 129 |
| | | DAEFRHDSGYE- | | 130 |
| | | VHHQKLVFFAE- | | |
| | | DVGSNKGAIIG | | |
| | | DAEFRHDSGYE- | 44 | 131 |
| | | VHHQKLVFFAE- | | |
| | | DVGSNKGAII | | |
| Neisseria meningitidis | PorA | YVAVENGVAKKVA | | 132 |
| | | HFVQQTPKSQPTLVP | | 133 |
| | | HVVVNNKVATHVP | | 134 |
| | | PLQNIQPQVTKR | | 135 |
| | | AQAANGGAASGQVKVTKVTKA | | 136 |
| | | YVDEQSKYHA | | 137 |
| | | HFVQNKQNQPPTLVP | | 138 |
| | | KPSSTNAKTGNKVEVTKA | | 139 |
| | | YWTTVNTGSATTTTFVP | | 140 |
| | | YVDEKKKMVHA | | 141 |
| | | HYTRQNNADVFVP | | 142 |
| | | YYTKDTNNNLTLVP | | 143 |
| | | PPQKNQSQPVVTKA | | 144 |
| | | PPSKGQTGNKVTKG | | 145 |
| | | PPSKSQPQVKVTKA | | 146 |
| | | QPQTANTQQGGKVKVTKA | | 147 |
| | | QPQVTNGVQGNQVKVTKA | | 148 |
| | | QPSKAQGQTNNQVKVTKA | | 149 |
| | | PPSSNQGKNQAQTGNTVTKA | | 150 |
| | | PPSKSQGKTGNQVKVTKA | | 151 |
| | | PPSKSQGTNNNQVKVTKA | | 152 |
| | | PPSKSQPGQVKVTKVTKA | | 153 |
| | | QLQLTEQPSSTNGQTGNQVKVTKA | | 154 |
| | | QLQLTEAPSKSQGAASNQVKVTKA | | 155 |
| | | SAYTPAHVYVDNKVAKHVA | | 156 |
| | | SAYTPAHFVQNKQNNNPTLVP | | 157 |
| | | VEGRNYQLQLTE | | 158 |
| | | PAQNSKSAYTPA | | 159 |
| | | QLQLTEPPSKNQAQTQNKVTKA | | 160 |
| | | GRDAFELFLLGSGSDE | | 161 |
| | | RHANVGRDAFELFLLGSGSDEA- | | 162 |
| | | KGTDPLKNH | | |
| | | GRDAFNLFLLGRIGDDDE | | 163 |
| | | GRNAFELFLIGSATSDQ | | 164 |
| | | QVKVTKAKSRIRTKI | | 165 |
| | | TLVPAVVGKPGSD | | 166 |
| | NspA | HAKASSSLGSAKGFSPR | | 167 |
| | | TRYKNYKAPSTDFKL | | 168 |
| | | SLNRASVDLGGSDSFSQT | | 169 |
| | | GKVNTVKNVRSGELSAGVRVK | | 170 |
| | | GKVNTVKNVRSGELSVGVRVK | | 171 |
| Immunoglobulin E | | APEWPGSRDKRTL | | 172 |
| | | EDGQVMDVD | | 173 |
| | | STTQEGEL | | 174 |
| | | GHTFEDSTKK | | 175 |
| | | GGGHFPPT | | 176 |
| | | PGTINI | | 177 |
| | | FTPPT | | 178 |
| | | INHRGYWV | | 179 |
| | | GEFCINHRGYWVCGDPA | | 180 |
| | | MAPEWPGSRDKRTL | | 181 |
| | | MEDGQVMDVD | | 182 |
| | | MSTTQEGEL | | 183 |
| | | MGHTFEDSTKK | | 184 |
| | | MGGGHFPPT | | 185 |
| | | MPGTINI | | 186 |
| | | MFTPPT | | 187 |
| | | MINHRGYWV | | 188 |
| | | MGEFCINHRGYWVCGDPA | | 189 |
| Hepatitis B | Surface | LQAGFFLLTR- | 48 | 190 |
| | | ILTIPQSLD- | | |
| | | SWWTSLNF | | |
| | PreS1 | MGTNLSVPN- | 37 | 191 |
| | | PLGFFPDHQLDP | | |
| | | PLGFFPDH | | 192 |
| | | PLGFFPDHQL | | 193 |
| | PreS2 | MQWNSTAFHQ- | 37 | 194 |
| | | TLQDPRVRG- | | |
| | | LYLPAGG | | |
| | | MQWSTAFHQ- | | 195 |
| | | TLQDP | | |
| | | MQWNSTALHQ- | | 196 |
| | | ALQDP | | |
| | | QDPRVR | 38 | 197 |
| | | DPRVRG- | 39 | 198 |
| | | LYLPAGG | | |
| | | DPRVRG- | 40 | 199 |
| | | LYFPAGG | | |
| B. anthracis | Protective Antigen | IVTKENTII- | 42 | 200 |
| | | NPSENGDTS- | | |
| | | TNGIEL | | |
| Hookworm | Asp-1 | IVYQHSHG- | 43 | 201 |
| | | EDRPGEL | | |
| Vesicular Stomatitis virus (VSV) | G glycoprotein | YTDIEMNRLGK | 48 | 202 |
| Bovine Respiratory Syncytial virus (BRSV) | F protein | DKELLPKVN- | 48 | 203 |
| | | NHDCQISN- | | |
| | | IATVIEFQQ | | |
| Human Respiratory Syncytial virus (RSV) | F protein | DKQLLPIVN- | 48 | 204 |
| | | KQSCSISN- | | |
| | | IETVIEFQQ | | |
| | | DKRLLPIVN- | 48 | 205 |
| | | QQSCRISN- | | |
| | | IETVIEFQQ | | |
| | | FPSDEF | 48 | 206 |
| Human Rhinovirus | VP1 | PATGIDNHREAKLD | 51 | 207 |
| Polio Virus | VP1 | SASTKNKDKL | 51 | 208 |

*Citations to published epitopes are provided following Table B.

For influenza A M2 polypeptide sequence X₁X₂X₃X₄X₅X₆X₇X₈TX₁₀X₁₁RX₁₃X₁₄X₁₅X₁₆X₁₇X₁₈X₁₉X₂₀X₂₁-X₂₂X₂₃X₂₄ of SEQ ID NO:72:

residues X₁ through X₈ are absent or present, and when present are the residues naturally present in the M2 protein sequence that are methionine, serine, leucine, leucine, threonine or proline, glutamic acid, valine, and glutamic acid, respectively, with the proviso that when one subscripted X residue is present, any remaining subscripted X with a higher subscript number up to 8 is also present,

X₁₀ is present and is proline, leucine or histidine,

X₁₁ is present and is isoleucine or threonine,

X₁₃ is present and is asparagine or serine,

X₁₄ is present and is glutamic acid or glycine,

residues X₁₅ and X₁₆ are present or absent, and when present are tryptophan and glycine or glutamic acid, respectively,

residues X₁₇ and X₁₉ are present or absent, and when present are independently cysteine, serine, or alanine,

residue X₁₈ is present or absent, and when present is arginine or lysine, and

residues X₂₀ through X₂₄ are present or absent, and when present are the residues naturally present in the M2 protein sequence that are asparagine or serine, aspartic acid or glycine, serine, serine and aspartic acid respectively, with the proviso that when one subscripted X residue is present, any remaining subscripted X residue with a lower subscript number down to 15 is also present.

Similarly, in the preferred above influenza A M2 sequence of SEQ ID NO: 70:

residues X₁ through X₈ are absent or present, and when present are the residues naturally present in the M2 protein sequence that are methionine, serine, leucine, leucine, threonine, glutamic acid, valine, and glutamic acid, respectively, with the proviso that when one subscripted X residue is present, any remaining subscripted X with a higher subscript number up to 8 is also present,

residues X₁₅ and X₁₆ are present or absent, and when present are tryptophan and glycine, respectively,

residues X₁₇ and X₉ are present or absent, and when present are independently cysteine, serine, or alanine,

residue X₁₈ is present or absent, and when present is arginine, and

residues X₂₀ through X₂₄ are present or absent, and when present are the residues naturally present in the M2 protein sequence that are asparagine, aspartic acid, serine, serine and aspartic acid respectively, with the proviso that when one subscripted X residue is present, any remaining subscripted X residue with a lower subscript number down to 15 is also present.

TABLE B
T Cell Epitopes

| | | | | SEQ |
|---|---|---|---|---|
| Organism | Gene | Sequence* | Citation | ID NO |
| HIV | P24 | GPKEPFRDY- | 3 | 209 |
| | | VDRFYKC | | |
| Corynebacterium | toxin | FQVVHNSYN- | 5 | 210 |
| diptheriae | | RPAYSPGC | | |
| Borrelia | ospA | VEIKEGTVTLKRE- | 6 | 211 |
| burgdorferi | | IDKNGKVTVSLC | | |
| | | TLSKNISKSG- | 7 | 212 |
| | | EVSVELNDC | | |
| Influenza Virus | HA | SSVSSFERFEC | 8 | 213 |
| A8/PR8 | | LIDALLGDPC | 32 | 214 |
| | | TLIDALLGC | 32 | 215 |
| | NP | FWRGENGRKTRS- | 36 | 216 |
| | | AYERMCNILKGK | | |
| | | LRVLSFIRGTKV- | 36 | 217 |
| | | SPRGKLSTRG | | |
| | | SLVGIDPFKLLQ- | 36 | 218 |
| | | NSQVYSLIRP | | |
| | | AVKGVGTMVMEL- | 36 | 219 |
| | | IRMIKRGINDRN | | |
| Trypanosoma | | SHNFTLVASVII- | 13 | 220 |
| cruzi | | EEAPSGNTC | | |
| Plasmodium | MSP1 | SVQIPKVPYPNGIVYC | 15 | 221 |
| falciparum | | DFNHYYTLKTGLEADC | | 222 |
| | | PSDKHIEQYKKI- | 23 | 223 |
| | | KNSISC | | |
| | | EYLNKIQNSLST- | 26 | 224 |
| | | EWSPCSVT | | |
| P. vivax | | YLDKVRATVGTE- | | 225 |
| | | WTPCSVT | | |
| P. yoelii | | EFVKQISSQLTE- | | 226 |
| | | EWSQCSVT | | |
| Streptococcus | AgI/II | KPRPIYEAKL- | 16 | 227 |
| sobrinus | | AQNQKC | | |
| | | AKADYEAKLA- | | 228 |
| | | QYEKDLC | | |
| LCMV (lymphocytic | NP | RPQASGVYM- | 17 | 229 |
| choriomeningitis virus) | | GNLTAQC | | |
| Clostridium | tox | QYIKANSKFIG- | 20 | 230 |
| tetani | | ITELC | | |
| Neisseria meningitidis | PorB | AIWQVEQKASIAGTDSGWC | | 231 |
| | | NYKNGGFFVQYGGAYKRHC | | 232 |
| | | HNSQTEVAATLAYRFGNVC | | 233 |
| | PorB | TPRVSYAHGFKGLVDDADC | | 234 |
| | | RFGNAVPRISYAHGFDFIC | | 235 |
| | | AFKYARHANVGRNAFELFC | | 236 |
| | | SGAWLKRNTGIGNYTQINAC | | 237 |
| | | AGEFGTLPAGRVANQC | | 238 |
| | | IGNYTQINAASVGLRC | | 239 |
| | | GRNYQLQLTEQPSRTC | | 240 |
| | | SGSVQFVPAQNSKSAC | | 241 |
| | | HANVGRDAFNLFLLGC | | 242 |
| | | LGRIGDDDEAKGTDPC | | 243 |
| | | SVQFVPAQNSKSAYKC | | 244 |
| | | NYAFKYAKHANVGRDC | | 245 |
| | | AHGFDFIERGKKGENC | | 246 |
| | | GVDYDFSKRTSAIVSC | | 247 |
| | | HDDMPVSVRYDSPDFC | | 248 |
| | | RFGNAVPRISYAHGFDFIERGKKGENC | | 249 |
| | | NYAFKYAKHANVGRDAFNLFLLGC | | 250 |
| | | SGAWLKRNTGIGNYTQINAASVGLRC | | 251 |
| | | SGSVQFVPAQNSKSAYTPAC | | 252 |
| | OpaB | TGANNTSTVSDYFRNRITC | | 253 |
| | | IYDFKLNDKFDKFKPYIGC | | 254 |
| | Opa-5d | LSAIYDFKLNDKFKPYIGC | | 255 |
| | Opac | NGWYINPWSEVKFDLNSRC | | 256 |
| Hepatitis B | Surface | MGTNLSVPN- | 37, 41 | 257 |
| | PreS1 | PLGFFPDHQLDP | | |
| | | PLGFFPDH | | 258 |
| | | PLGFFPDHQL | | 259 |
| | PreS2 | MQWNSTAFHQ- | 37 | 260 |
| | | TLQDPRVRG- | | |
| | | LYLPAGG | | |
| | | MQWSTAFHQ- | | 261 |
| | | TLQDP | | |
| | | MQWNSTAIJHQ- | | 262 |
| | | ALQDP | | |
| | | QDPRVR | 38 | 263 |
| | Core | MDIDPYKEFGAT- | 45 | 264 |
| | | VELLSFLP | | |
| | | RDLLDTASALYR- | 45 | 265 |
| | | EALESPEHCSPHH | | |
| | | TWVGVNLEDPAS- | 45 | 266 |
| | | RDLVVSYVNTNMG | | |
| | | VVSYVNTNMGL- | 45 | 267 |
| | | KFRQL | | |
| | | LLWFHISCLTF- | 45 | 268 |
| | | GRETVIEYLV | | |
| | | LLWFHISCLTF- | 45 | 269 |
| | | VSFGVWIRTPP- | 45 | 270 |
| | | AYRPPNAPIL | | |
| | | VSFGVWIRTPPA | 45 | 271 |
| | | PPAYRPPNAPIL | 45 | 272 |
| | | WIRTPPAYRPPN | 45 | 273 |
| | | PHHTALRQAIL- | 46 | 274 |
| | | CWGELMTLA | | |

*Underlined C (C) is not from the native sequence.
Citations:
1. EPO 786 521A.
2. WO 98/07320.
3. US No. 5,639,854.
4. US No. 4,544,500.
5. EPO 399001 B1.
6. Bockenstedt et al. (1996) J. Immunol., 157, 12: 5496.
7. Zhong et al. (1996) Eur. J. Immunol., 26, 11: 2749.
8. Brumeanu et al. (1996) Immunotechnology, 2, 2: 85.
9. Hill et al. (1997) Infect. Immun., 65, 11: 4476.
10. EPO 432 220 B1.
11. WO 98/06851.
12. Kelly et al. (1997) Clin. Exp. Immunol., 110, 2: 285.
13. Kahn et al. (1997) J. Immunol., 159, 9: 4444.
14. WO 97/18475.
15. Ohta et al. (1997) Int. Arch. Allergy Immunol., 114, 1: 15.
16. Staffileno et al. (1990) Arch. Oral Biol., 35: Suppl. 47S.
17. Saron et al. (1997) Proc. Natl. Acad. Sci. USA, 94, 7: 3314.
18. Corthesy et al. (1996) J. Biol. Chem., 271, 52: 33670.
19. Bastien et al. (1997) Virol., 234, 1: 118.
20. Yang et al. (1997) Vaccine, 15, 4: 377.
21. Lotter et al. (1997) J. Exp. Med., 185, 10: 1793.
22. Nara et al. (1997) Vaccine 15, 1: 79.
23. U.S. No. 4,886,782.
24. Zavala et al. (1985) Science, 228: 1436.
25. Schodel et al. (1994) J. Exper. Med., 180: 1037.
26. Calvo-Calle et al. (1997) J. Immunol. 159, 3: 1362.
27. Qari et al. (1992) Mol. Biochem. Parasitol., 55(1-2): 105.
28. Qari et al. (1993) Lancet, 341(8848): 780.
29. Neirynck et al. (October 1999) Nature Med., 5(10): 1157-1163.
30. Thompson et al. (1994) Eur. J. Biochem., 226(3): 751-764.
31. Wilson et al. (2000) Science, 287: 1664-1666.
32. Brown et al. (1993) J. Virol., 67(5): 2887-2893.
33. U.S. No. 4,886,663.
34. Schenk et al. (Jul. 8, 1999) Nature, 400(6740): 116-117.
35. Slepushkin et al. (1995) Vaccine, 13(15): 1399-1402.
36. Brett et al., (1991) J. Immunol., 147(3): 984-991.
37. Neurath et al., (1986) F. Brown et al. eds., Vaccines 85, Cold Spring Harbor Laboratory, Cold Spring Harbor, New York, pp.185-189.
38. Kent et al., (1987) F. Brown et al. eds., Vaccines 86, Cold Spring Harbor Laboratory, Cold Spring Harbor, New York, pp.365-369.
39. Milich et al., (1987) F. Brown et al. eds., Vaccines 86, Cold Spring Harbor Laboratory, Cold Spring Harbor, New York, pp.377-382.
40. Thornton et al., (1987) F. Brown et al. eds., Vaccines 87, Cold Spring Harbor Laboratory, Cold Spring Harbor, New York, pp.77-80.
41. Milich et al., (1987) F. Brown et al. eds., Vaccines 87, Cold Spring Harbor Laboratory, Cold Spring Harbor, New York, pp.50-55.
42. Little et al., (1996) Microbiology 142: 707-715.
43. Provided by Dr. Peter Hotez, George Washington University.
44. Morgan et al., (2000) Nature 408: 982-985.
45. U.S. Pat. No. 4,882,145.
46. Alexander et al., (1994) Immunity 1: 751-761.
47. Muyanga et al., (2001) Arch. Virol. 146(9): 1667-1679.
48. U.S. Pat. No. 6,171,591.
49. U.S. Pat. No. 6,060,064.
50. U.S. Pat. No. 6,110,466.

Another useful T cell epitope is a synthetic sequence referred to as a PADRE epitope (See, U.S. Pat. No. 6,413,517 to Sette et al.) One exemplary epitope is that disclosed by Alexander et al., (1994) Immunity, 5:751-761 that has the sequence AKFVAAWTLKAAA (SEQ ID NO: 275).

In certain embodiments, the epitope comprises a haptenic small drug molecule, a derivative or an analogue thereof. In some embodiments the epitope is a drug of abuse, analogue or derivative of a drug of abuse such as nicotine, ethanol, cocaine, heroin, morphine, fentanyl, methylfentanyl, amphetamine, methamphetamine, phencyclidine, methylphenidate and methylenedioxymethamphetamine. Exemplary of such materials are the cocaine analogs described in U.S. Pat. No. 6,383,490 that are adapted for use as covalently linked to an immunogenic carrier.

In other embodiments, the epitope is a saccharide. Illustrative saccharide compounds include lipooligosaccharides (LOS) of NTHi or M. cat. [Sun et. al. (2000) Vaccine 2000, 18(13):1264-1272; and Jiao et. al. (2002) Infect. Immun., 70(11):5982-5989]. LOS consist of a hydrophobic lipid A portion and a hydrophilic core oligosaccharide portion and are one of the major components in the outer membranes of gram-negative bacteria. Detoxified LOS (dLOS) molecules are de-O-acylated or de-N-acylated, or both, and exhibit about 100- to about 10,000-fold diminished endotoxicity compared with the starting LOS in the Limulus amoebocyte lysate (LAL) assay as described in Hochstein (1990) in Clinical application of the Limulus amoebocyte lysate test, R. B. Prior, ed. CRC Press, Inc., Boca Raton, Fla., pages 38-49.

There are many methods known in the art to couple carrier proteins to polysaccharides. Aldehyde groups can be prepared on either the reducing end [Anderson (1983) Infect. Immun., 39:233-238; Jennings et al. (1981) J. Immunol., 127:1011-1018; Poren et al. (1985) Mol. Immunol., 22:907-919] or the terminal end [Anderson et al. (1986) J. Immunol., 137:1181-1186; Beuvery et al. (1986) Dev. Bio. Scand., 65:197-204] of an oligosaccharide or relatively small polysaccharide, which can be linked to the carrier protein via reductive amination. In addition, oxidation of adjacent hydroxyls by periodate ion followed by reductive alkylation of an amine using cyanoborohydride is particularly useful for those HBc chimer molecules having a added E-amine-supplying lysine residue added into the immunogenic loop of the chimer.

Large polysaccharides can be conjugated by either terminal activation [Anderson et al. (1986) J. Immunol., 137:1181-1186] or by random activation of several functional groups along the polysaccharide chain [Chu et al. (1983) Infect. Immun., 40:245-256; Gordon, U.S. Pat. No. 4,619,828 (1986); Marburg, U.S. Pat. No. 4,882,317 (1989)]. Random activation of several functional groups along the polysaccharide chain can lead to a conjugate that is highly cross-linked due to random linkages along the polysaccharide chain. The optimal ratio of polysaccharide to carrier protein depends on the particular polysaccharide, the carrier protein, and the conjugate used.

Detailed reviews of methods of conjugation of saccharide to carrier proteins can be found in Dick et al., in Contributions to Microbiology and Immunology, Vol. 10, Cruse et al., eds., (S. Karger: 1989), pages 48-114; Jennings et al., in Neoglycoconjugates: Preparation and Applications, Lee et al., eds., (Academic Press: 1994), pages 325-371; Aplin et al., (1981) CRC Crit. Rev. Biochem., 10:259-306; and Stowell et al. (1980) Adv. Carbohydr. Chem. Biochem., 37:225-281.

The carbohydrate itself can be synthesized by methods known in the art, for example by enzymatic glycoprotein synthesis as described by Witte et al. (1997) J. Am. Chem. Soc., 119:2114-2118.

Several oligosaccharides, synthetic and semi-synthetic, and natural, are discussed in the following paragraphs as examples of oligosaccharides that are contemplated haptens to be used in making a HBc conjugate of the present invention.

An oligosaccharide hapten suitable for preparing vaccines for the treatment of Haemophilus influenza type b (Hib) is made up of from 2 to 20 repeats of D-ribose-D-ribitol-phosphate (I, below), D-ribitol-phosphate-D-ribose (II, below), or phosphate-D-ribose-D-ribitol (III, below). Eduard C. Beuvery et al., EP-0 276 516-B1.

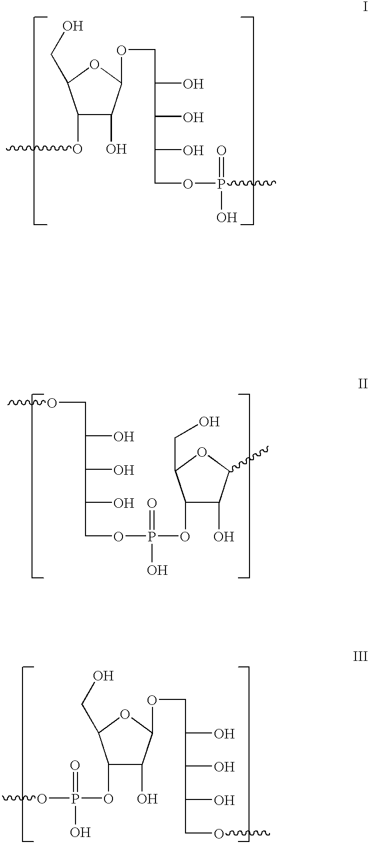

U.S. Pat. No. 4,220,717 also discloses a polyribosyl ribitol phosphate (PRP) hapten for Haemophilus influenzae type b.

Peterson et al. (1998) Infect. Immun., 66(8):3848-3855, disclose a trisaccharide hapten, αKdo(2→8)αKdo(2→4)αKdo, that provides protection from Chlamydia pneumoniae. Chlamydia pneumoniae is a cause of human respiratory infections ranging from pharyngitis to fatal pneumonia. Kdo is 3-deoxy-D-manno-oct-2-ulosonic acid.

Andersson et al., EP-0 126 043-A1, disclose saccharides that can be used in the treatment, prophylaxis or diagnosis of bacterial infections caused by Streptococci pneumoniae. One class of useful saccharides is derived from the disaccharide GlcNAcβ1→3Gal. Andersson et al., above, also reported neolactotetraosylceramide to be useful, which is Galβ1→4GlcNAcβ1→3Galβ1→4Glc-Cer.

McKenney et al. (1999) Science, 284:1523-1527, disclose a polysaccharide, poly-N-succinyl β1→6GlcN (PNSG) that provides protection from Staphylococcus aureus. S. aureus is a common cause of community-acquired infections, including endocarditis, osetemylitis, septic arthritis, pneumonia, and abscesses.

European Patent No. 0 157 899-B1 discloses the isolation of pneumococcal polysaccharides that are useful in the present invention. The following table lists the pneumococcal culture types that produce capsular polysaccharides useful as haptens in the present invention.

Polysaccharide Hapten Sources

| Danish Type | U.S. | 1978 ATCC Catalogue |
|---|---|---|
| Nomenclature | Nomenclature | Number |
| 1 | 1 | 6301 |
| 2 | 2 | 6302 |
| 3 | 3 | 6303 |
| 4 | 4 | 6304 |
| 5 | 5 | |
| 6A | 6 | 6306 |
| 6B | 26 | 6326 |
| 7F | 51 | 10351 |
| 8 | 8 | 6308 |
| 9N | 9 | 6309 |
| 9V | 68 | |
| 10A | 34 | |
| 11A | 43 | |
| 12F | 12 | 6312 |
| 14 | 14 | 6314 |
| 15B | 54 | |
| 17F | 17 | |
| 18C | 56 | 10356 |
| 19A | 57 | |
| 19F | 19 | 6319 |
| 20 | 20 | 6320 |
| 22F | 22 | |
| 23F | 23 | 6323 |
| 25 | 25 | 6325 |
| 33F | 70 | |

Moraxella (Branhamella) catarrhalis is a reported cause of otitis media and sinusitis in children and lower respiratory tract infections in adults. The lipid A portion of the lipooligo-saccharide surface antigen (LOS) of the bacterium is cleaved at the 3-deoxy-D-manno-octulosonic acid-glucosamine linkage. The cleavage product is treated with mild-alkali or hydrazine to remove ester-linked fatty acids, while preserving amide-linked fatty acids to yield detoxified lipopolysaccharide (dLOS) from M. catarrhalis. The dLOS is not immunogenic until it is attached to a protein carrier. Xin-Xing Gu et al. (1998) Infect. Immun., 66(5):1891-1897.

Group B streptococci (GBS) is a cause of sepsis, meningitis, and related neurologic disorders in humans. The Capsular polysaccharide-specific antibodies are known to protect human infants from infection. Jennings et al., U.S. Pat. No. 5,795,580. The repeating unit of the GBS capsular polysaccharide type II is: →4)-β-D-GlcpNAc-(1→3)-[β-D-Galp(1→6)]-β-D-Galp(1→4)-β-D-Glcp-(1→3)-β-D-Glcp-(1→2)-[α-D-NeupNAc(2→3)]-β-D-Galp-(1→, where the bracketed portion is a branch connected to the immediately following unbracketed subunit. The repeating unit of GBS capsular polysaccharide type V is: →4)-[α-D-NeupNAc-(2→3)-β-D-Galp-(1→4)-β-D-GlcpNAc-(1→6)]-α-D-Glcp-(1→4)-[β-D-Glcp-(1→3)]-β-D-Galp-(1→4)-β-D-Glcp-(1→.

European patent application No. EU-0 641 568-A1, Brade, discloses the method of obtaining ladder-like banding pattern antigen from Chlamydia trachomatis, pneumoniae and psittaci.

Slovin et al., (1999) Proc. Natl. Acad. Sci., U.S.A., 96(10):5710-5715 report use of a synthetic oligosaccharide, globo H, linked to KLH as a carrier in the preparation of a vaccine used against prostate cancer. Similarly, Helling et al., (July 1995) Cancer Res., 55:2783-2788 report the use of KLH-linked G_{M2} in a vaccine for treating patients with melanoma. The latter vaccine was prepared by ozone cleavage of the ceramide double bond of G_{M2}, introduction of an aldehyde group and reductive alkylation onto KLH. A similar procedure can be utilized with a contemplated chimer particle.

Oligosaccharidal portions of sphingolipids such as globosides and gangliosides that are present on the surface of other tumor cells as well as normal cells such as melanoma, neuroblastoma and healthy brain cells can similarly be used herein as a hapten. The oligosaccharide portion of the globoside globo H has the structure Fucα-(1→2)-Galβ(1→3)-GalNAcβ-(1→3)-Galβ-(1→4)-Galβ-(1→4)Glc, whereas the saccharide protions of gangliosides G_{M2}, G_{M1} and G_{D1a} have the following structures: GalNAcβ-(1→4)-[NeuAcα-(2→3)]-Galβ-(1→4)-Glc; Galβ-(1→3)-GalNAcβ-(1→4)-[NeuAcα-(2→3)]-Galβ-(1→4)-Glc; and NeuAc-(2→3)-Galβ-(1→3)-GalNAcβ-(1→4)-[NeuAcα-(2→3)]-Galβ-(1→4)-Glc, respectively.

U.S. Pat. No. 4,356,170 discloses the preparation of useful polysaccharides that are reduced and then oxidized to form compounds having terminal aldehyde groups that can be reductively aminated onto free amine groups of carrier proteins such as tetanus toxoid and diphtheria toxoid with or without significant cross-linking. Exemplary useful bacterial polysaccharides include β-hemolytic streptococci, Haemophilus influenza, meningococci, pneumococci and E. coli. Rather than reductively aminating the particles, a linker arm such as that provided by an ε-amino C₂-C₈ alkylcarboxylic acid can be reductively aminated on to the polysaccharide, followed by linkage to the particles using a water-soluble carbodiimide.

Additionally useful sequences that are neither B cell nor T cell epitopes bind to biotin and can be expressed as part of a chimeric polypeptide that self-assembles into a VLP. Two such sequences are disclosed in U.S. Pat. No. 6,380,364 as GGGCSWAPPFKASC (SEQ ID NO:276) and GGGRGEFTGTYITAVT (SEQ ID NO:277). These two sequences can similarly be coexpressed as a fusion polypeptide with a previously discussed VLP-forming polypeptide.

A further preferred embodiment is a method for stabilizing VLPs. VLPs are stabilized by attaching a self-binding peptide second portion to a self-assembling polypeptide first portion by a covalent bond. In some embodiments the attachment is made through a peptide bond. In some embodiments the attachment via a peptide bond is accomplished by genetically fusing DNA encoding a self-binding peptide portion to the DNA coding sequence of a self-assembling polypeptide second portion. In certain embodiments the first and second portions are attached in vitro, for example by way of a bifunctional linking reagent or by protein ligation. Yet another embodiment of this invention is a method for stabilizing VLPs to which heterologous epitopes may be attached by one or more covalent or non-covalent bonds.

It is appreciated by those skilled in the art that stabilized VLPs may have applications in technologies outside the medical field. It is appreciated by those skilled in the art that epitopes bound to stabilized VLPs can also have applications in technologies outside the medical field. See for example Mao et al., Proc. Nat. Acad. Sci. (2003) 100:6946-51. See also Wang (2002) Angew. Chem. Int. Ed. 41:459-62, where engineered virions are described as “addressable nanoscale building blocks”.

It is appreciated by those skilled in the art that production of the invention in a biological system (e.g., one or more portions of the inventive chimeric polypeptide being expressed via recombinant DNA encoding the polypeptide(s) when present in a host-appropriate expression vector introduced into an appropriate host cell (as described elsewhere) is only one way of producing any or all portions of the chimeric polypeptide, a stabilizing peptide, and/or an epitope, and is used in examples that follow only for convenience. In vitro protein ligation and other chemical techniques known in the art can be employed to build, via covalent bonds, the inventive polypeptides and VLPs entirely from in vitro synthesized polypeptides and other precursors. [See e.g., Muir et al., (1998) Proc. Natl. Acad. Sci., 95:6705-6710 for semisynthetic methods; Canne et al. (1995) J. Am. Chem. Soc., 117:2998-3004 for total synthesis of a multi-subunit polyprotein.]

It will also be clear to one skilled in the art that some embodiments will not be possible to construct in vivo or in all cell types. For example, small molecules are attached in vitro to a linker residue on the chimeric polypeptide, as taught by for example by Carerra et al. (2000) Proc. Natl. Acad. Sci., 98:1988-1992.

Persons having skill in the art will recognize that the particular examples above of covalent and non-covalent attachment of the epitope to the first portion of the chimeric capsid polypeptide are merely exemplary and that many other methods of attachment are known in the art and are readily adaptable for making such attachments in vivo or in vitro.

An analogue or analogous nucleic acid (DNA or RNA) sequence that encodes a contemplated chimeric polypeptide or a portion of a chimeric polypeptide (a “chimer analogue”) is also contemplated as part of this invention. A chimer analog (or analogue) nucleic acid sequence or its complementary nucleic acid sequence encodes an amino acid residue sequence that is at least 50 percent, more preferably 70 percent, even more preferably 80 percent, and even more preferably at least 90 percent, and most preferably is at least 95 percent identical to a first portion of a chimeric polypeptide. Such a DNA or RNA is referred to herein as an “analog (analogue) of” or “analogous to” a sequence of a nucleic acid encoding a viral capsid polypeptide or self-assembling polypeptide (including but not limited to those enumerated in the examples below). A nucleic acid that encodes an analogous sequence, upon suitable transfection and expression, also produces a first portion of a contemplated chimeric polypeptide.

One can exploit the degeneracy of the genetic code to encode a first portion of a chimeric polypeptide that avoids substantial identity with, for example, a genomic virus capsid polypeptide gene or gene fragment encoding the same amino acid sequence or its complement. Thus, a useful analogous DNA sequence need not, for example, hybridize with the nucleotide sequence of the virus capsid polypeptide coding sequence or a complement under conditions of moderate stringency, but can still provide a contemplated chimer molecule; that is, many nucleic acid sequences can encode the same polypeptide. For these and reasons that follow, the most appropriate measure of similarity is between amino acid sequences rather than nucleic acid sequences.

Different host organisms have different codon preferences, that is, they preferentially use a particular codon to encode a particular amino acid residue. Such codon preferences are well known and a DNA sequence encoding a desired chimer sequence can be altered using in vitro mutagenesis, for example, so that host-preferred codons are utilized when a polypeptide is to be expressed. Such alteration is routine in the art since optimized coding sequences result in higher quantity and quality of protein to be expressed from a particular expression host.

A recombinant nucleic acid molecule such as a DNA molecule, comprising a vector operatively linked to an exogenous nucleic acid segment (e.g., a DNA segment or sequence) that defines a gene that encodes a portion of a contemplated chimeric polypeptide, as discussed above, and a promoter suitable for directing the expression of the gene in a compatible host organism, is also contemplated in this invention. More particularly, also contemplated is a recombinant DNA molecule that comprises a vector containing a promoter directing the expression of a portion of a contemplated chimeric polypeptide in host organism cells operatively linked to a DNA segment that defines a gene for the first portion of a chimeric polypeptide or a DNA variant that has at least 50 percent amino acid identity to the contemplated chimer polypeptide.

Further contemplated is a recombinant DNA molecule that comprises a vector containing a promoter for controlling the expression of a chimeric polypeptide portion in host organism cells operatively linked to a DNA segment that is an analog nucleic acid sequence that encodes an amino acid residue sequence of a first portion that is at least 50 percent identical, more preferably 70 percent identical, more preferably 80 percent identical, more preferably 90 percent identical, and most preferably at least 95 percent identical to the first portion of a sequence of a contemplated chimeric polypeptide. That recombinant DNA molecule, upon suitable transfection and expression by a host cell, provides a contemplated chimer molecule.

It is noted that some embodiments of the first polypeptide portion of the invention include heterologous epitope amino acid residues or sequences and/or linker residues or sequences. Those heterologous sequences and/or their encoding nucleic acid sequences and/or their complements are not included in the above percentages and comparisons of sequence identity. Similarly, sequences that are truncated from a first portion of a chimeric polypeptide relative to, e.g., a virus capsid polypeptide from which it is ultimately derived, e.g., N- or C-terminal sequences to abrogate nucleic acid binding, are not included in identity calculations and comparisons. Thus, only bases or residues that are present in a first portion of a chimer molecule, exclusive of heterologous linkers and/or epitopes are included and compared to aligned nucleic acid or amino acid residue sequences in the identity percentage calculations and comparisons.

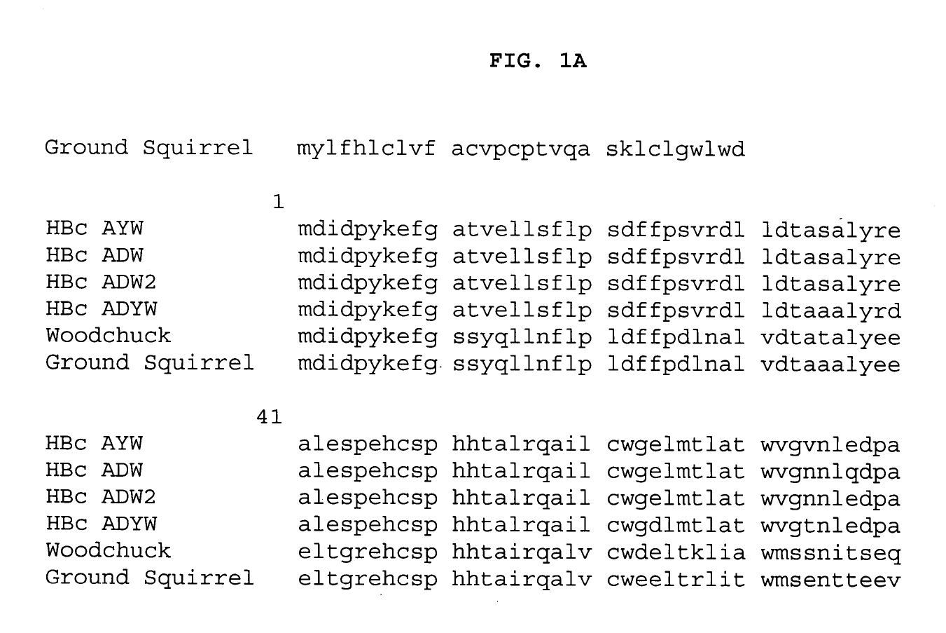

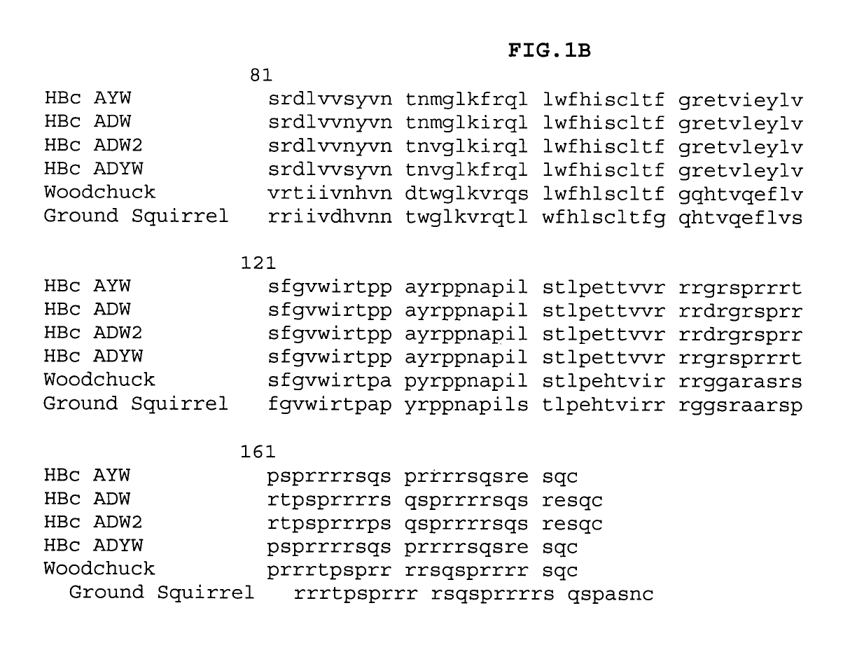

The coding sequences for the genes of illustrative hepatitis B virus-derived VLPs are illustrated in SEQ ID NOs: 3, 4, 5, 6, 7 and 8 of FIG. 1. Isolated nucleic acid segments, preferably DNA sequences, variants and analogs thereof can be prepared by in vitro mutagenesis, as is well known in the art and discussed in Current Protocols In Molecular Biology, Ausabel et al. eds., John Wiley & Sons (New York: 1987) p. 8.1.1-8.1.6, that begin at the initial ATG codon for a gene and end at or just downstream of the stop codon for each gene. Thus, a desired restriction site can be engineered at or upstream of the initiation codon, and at or downstream of the stop codon so that other genes can be prepared, excised and isolated. Using methods well-known in the art, then, genetic fusion and/or operational linkage of essentially any set of nucleic acid sequences is technically feasible.

As is well known in the art, so long as the required nucleic acid, illustratively DNA sequence, is present, (including start and stop signals), additional base pairs can usually be present at either end of the segment and that segment can still be utilized to express the protein. This, of course, presumes the absence in the segment of an operatively linked DNA sequence that represses expression, expresses a further product that consumes the desired expression product, expresses a product that consumes a wanted reaction product produced by a desired enzyme, or otherwise interferes with expression or function of the gene of the DNA segment.

Thus, so long as a DNA segment is free of such interfering DNA sequences, a DNA segment of the invention can be about 500 to about 100,000 base pairs in length, including vector sequences. The maximum size of a recombinant DNA molecule, particularly an expression vector, is governed mostly by convenience and the vector size that can be accommodated by a host cell, once all of the minimal DNA sequences required for replication and expression, when desired, are present. Minimal expression vectors and their sizes are well known. Long DNA segments and large vectors are not preferred, but can be used.

As noted DNA segments that encode the before-described chimeric polypeptide portion or analogue can be synthesized entirely by non-biological chemical techniques, for example, the phosphotriester method of Matteucci et al. (1981) J. Am. Chem. Soc., 103:3185. Of course, by chemically synthesizing a genetic sequence, any desired modifications can be made simply by substituting the appropriate bases for those encoding the native amino acid residue sequence. However, DNA segments including sequences discussed previously are preferred.

A contemplated chimeric polypeptide can be produced (expressed) in a number of transformed host systems, typically host cells, although expression in acellular, in vitro, systems is also contemplated. These host cellular systems include, but are not limited to, microorganisms such as bacteria transformed with recombinant bacteriophage, plasmid, or cosmid DNA expression vectors; yeast transformed with yeast expression vectors; plant, animal or bacterial cell systems transfected with virus expression vectors (e.g. cauliflower mosaic virus; tobacco mosaic virus; alpha viruses; baculovirus; fd) or transformed using bacterial expression vectors (e.g., Ti plasmid); or appropriately transformed animal cell systems such as CHO, VERO or COS cells. As is also noted below, the invention is neither limited nor defined by the host cell system employed, nor by the nature of the genetic elements controlling and directing expression.

DNA segments containing a gene encoding, e.g., a HB capsid polypeptide are preferably obtained from recombinant DNA molecules (plasmid vectors) containing that gene. Vectors capable of directing the expression of such a gene as to result in transcription of the sequence (and for encoded polypeptides, as here, their subsequent translation) is referred to herein as an “expression vector”.

An expression vector contains expression control elements including a promoter. The chimeric polypeptide portion-encoding sequence is operatively linked to the expression vector to permit the promoter sequence to direct RNA polymerase binding and expression of the chimeric polypeptide portion-encoding gene. Useful in expressing the polypeptide coding gene are promoters that are inducible, viral, synthetic, constitutive as described by Poszkowski et al. (1989) EMBO J., 3:2719 and Odell et al. (1985) Nature, 313:810, as well as temporally regulated, spatially regulated, and spatiotemporally regulated as given in Chua et al. (1989) Science, 244:174-181.

One preferred promoter for use in prokaryotic cells such as E. coli is the Rec 7 promoter that is inducible by exogenously supplied nalidixic acid. A more preferred promoter is present in plasmid vector JHEX25 (available from Promega Corp., Madison, Wis.) that is inducible by exogenously supplied isopropyl-β-D-thiogalacto-pyranoside (IPTG). A still more preferred promoter, the tac promoter, is present in plasmid vector pKK223-3 and is also inducible by exogenously supplied IPTG. The pKK223-3 plasmid can be successfully expressed in a number of E. coli strains, such as XL-1, TB1, BL21 and BLR, using about 25 to about 100 μM IPTG for induction. Surprisingly, concentrations of about 25 to about 50 μM IPTG have been found to provide optimal results in 2 L shaker flasks and fermentors. Other promoters and genetic regulatory elements, as is well known in the art, can be required for expression in other cell types and organisms.

A variety of methods have been developed to operatively link DNA to vectors via complementary cohesive termini or blunt ends. For instance, complementary homopolymer tracts can be added to the DNA segment to be inserted into the vector DNA. The vector and DNA segment are then joined by hydrogen bonding between the complementary homopolymeric tails to form recombinant DNA molecules.

Alternatively, synthetic linkers containing one or more restriction endonuclease sites can be used to join the DNA segment to the expression vector, as noted before. The synthetic linkers are attached to blunt-ended DNA segments by incubating the blunt-ended DNA segments with a large excess of synthetic linker molecules in the presence of an enzyme that is able to catalyze the ligation of blunt-ended DNA molecules, such as bacteriophage T4 DNA ligase. Thus, the products of the reaction are DNA segments carrying synthetic linker sequences at their ends. These DNA segments are then cleaved with the appropriate restriction endonuclease and ligated into an expression vector that has been cleaved with an enzyme that produces termini compatible with those of the synthetic linker. Synthetic linkers containing a variety of restriction endonuclease sites are commercially available from a number of sources including New England BioLabs, Beverly, Mass.

A desired DNA segment can also be obtained using PCR technology in which the forward and reverse primers contain desired restriction sites that can be cut after amplification so that the gene can be inserted into the vector. Alternatively PCR products can be directly cloned into vectors containing T-overhangs (Promega Corp., A3600, Madison, Wis.) as is well known in the art.

It is well known in the art that almost any desired genetic sequence can be produced by in vitro synthesis, PCR amplification and manipulation, site-directed mutagenesis, or a combination of these or other techniques known in the art. Indeed, wholly synthetic, customized genetic constructs may be commercially procured (e.g., from Aptagen, Herndon, Va. USA, Sigma-Genosys The Woodlands, Tex. USA, GenScript Corp. Edison, N.J. USA, and many others). Further, different host organisms use distinct genetic sequences for control and direction of gene expression (in addition to codon preferences mentioned above), and so it is contemplated that expression vectors encoding an inventive chimeric polypeptide or portion thereof can be adapted to one or more of numerous possible host organisms, and yet remain within the scope of the claimed invention.

Considering the combination of the degeneracy of the genetic code, distinct codon preferences among organisms, the commercial availability of fully customized genetic constructs, the diverse systems for protein expression from genetic constructs, and other genetic variations both natural and non-natural in origin, it is seen that the most functionally meaningful determination of similarity between two polypeptides or their encoding genes is that of the encoded amino acid sequence rather than nucleic acids that encode them.

Inocula and Vaccines

In yet another embodiment of the invention, a HBc chimer particle or HBc chimer particle conjugate with a hapten is used as the immunogen of an inoculum or vaccine in an human patient or suitable animal host such as a chimpanzee, mouse, rat, horse, sheep or the like. An inoculum can induce a B cell or T cell response (stimulation) such as production of antibodies that immunoreact with the immunogenic epitope or hapten, or T cell activation, whereas a vaccine provides protection against the entity from which the immunogen has been derived via one or both of a B cell or T cell response.

T cell activation can be measured by a variety of techniques. In usual practice, a host animal is inoculated with a contemplated HBc chimer particle vaccine or inoculum, and peripheral mononuclear blood cells (PMBC) are thereafter collected. Those PMBC are then cultured in vitro in the presence of the T cell immunogen for a period of about three to five days. The cultured PMBC are then assayed for proliferation or secretion of a cytokine such as IL-2, GM-CSF of IFN-γ. Assays for T cell activation are well known in the art. See, for example, U.S. Pat. No. 5,478,726 and the art cited therein.

Using antibody formation as exemplary, a contemplated inoculum or vaccine comprises an immunogenically effective amount of HBc chimer particles or HBc chimer particle conjugates that are dissolved or dispersed in a pharmaceutically acceptable diluent composition that typically also contains water. When administered to a host animal in need of immunization or in which antibodies are desired to be induced such as a mammal (e.g., a mouse, dog, goat, sheep, horse, bovine, monkey, ape, or human) or bird (e.g., a chicken, turkey, duck or goose), an inoculum induces antibodies that immunoreact with the genetically linked or conjugated (pendently-linked) hapten. Those antibodies also preferably bind to the protein or saccharide of the B cell immunogen.

The amount of recombinant HBc chimer immunogen utilized in each immunization is referred to as an immunogenically effective amount and can vary widely, depending inter alia, upon the recombinant HBc chimer immunogen, patient immunized, and the presence of an adjuvant in the vaccine, as discussed below. Immunogenically effective amounts for a vaccine and an inoculum provide the protection or antibody activity, respectively, discussed hereinbefore.

Vaccines or inocula typically contain a recombinant HBc chimer immunogen concentration of about 1 microgram to about 1 milligram per inoculation (unit dose), and preferably about 10 micrograms to about 50 micrograms per unit dose. The term “unit dose” as it pertains to a vaccine or inoculum of the present invention refers to physically discrete units suitable as unitary dosages for animals, each unit containing a predetermined quantity of active material calculated to individually or collectively produce the desired immunogenic effect in association with the required diluent; i.e., carrier, or vehicle.

Vaccines or inocula are typically prepared from a recovered recombinant HBc chimer immunogen by dispersing the immunogen, preferably in particulate form, in a physiologically tolerable (acceptable) diluent vehicle such as water, saline, phosphate-buffered saline (PBS), acetate-buffered saline (ABS), Ringer's solution, or the like to form an aqueous composition. The diluent vehicle can also include oleaginous materials such as peanut oil, squalane, or squalene as is discussed hereinafter.

The preparation of inocula and vaccines that contain proteinaceous materials as active ingredients is also well understood in the art. Typically, such inocula or vaccines are prepared as parenterals, either as liquid solutions or suspensions; solid forms suitable for solution in, or suspension in, liquid prior to injection can also be prepared. The preparation can also be emulsified, which is particularly preferred.

The immunogenically active ingredient is often mixed with excipients that are pharmaceutically acceptable and compatible with the active ingredient. Suitable excipients are, for example, water, saline, dextrose, glycerol, ethanol, or the like and combinations thereof. In addition, if desired, an inoculum or vaccine can contain minor amounts of auxiliary substances such as wetting or emulsifying agents, pH buffering agents that enhance the immunogenic effectiveness of the composition.

A contemplated vaccine or inoculum advantageously also includes an adjuvant. Suitable adjuvants for vaccines and inocula of the present invention comprise those adjuvants that are capable of enhancing the antibody responses against B cell epitopes of the chimer, as well as adjuvants capable of enhancing cell mediated responses towards T cell epitopes contained in the chimer. Adjuvants are well known in the art (see, for example, Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X).

Exemplary adjuvants include complete Freund's adjuvant (CFA) that is not used in humans, incomplete Freund's adjuvant (IFA), squalene, squalane and alum [e.g., Alhydrogel™ (Superfos, Denmark)], which are materials well known in the art, and are available commercially from several sources.

Preferred adjuvants for use with immunogens of the present invention include aluminum or calcium salts (for example hydroxide or phosphate salts). A particularly preferred adjuvant for use herein is an aluminum hydroxide gel such as Alhydrogel™. For aluminum hydroxide gels, the chimer protein is admixed with the adjuvant so that between 50 to 800 micrograms of aluminum are present per dose, and preferably between 400 and 600 micrograms are present. Another particularly preferred adjuvant is aluminum phosphate that is available under trademark Adju-Phos™ from Superfos Biosector, Denmark. Primary aluminum phosphate particles have a plate-like morphology and a diameter of about 50 to about 100 nm, with the final particle size in the product being about 0.5 to about 10μ. Calcium phosphate nanoparticles (CAP) are an adjuvant being developed by Biosante, Inc (Lincolnshire, Ill.). The immunogen of interest can be either coated to the outside of particles, or encapsulated inside on the inside ([He et al., (November 2000) Clin. Diagn. Lab. Immunol., 7(6):899-903].

Another particularly preferred adjuvant for use with an immunogen of the present invention is an emulsion. A contemplated emulsion can be an oil-in-water emulsion or a water-in-oil emulsion. In addition to the immunogenic chimer protein, such emulsions comprise an oil phase of squalene, squalane, peanut oil or the like as are well-known, and a dispersing agent. Non-ionic dispersing agents are preferred and such materials include mono- and di-C₁₂-C₂₄-fatty acid esters of sorbitan and mannide such as sorbitan mono-stearate, sorbitan mono-oleate and mannide mono-oleate. An immunogen-containing emulsion is administered as an emulsion.

Preferably, such emulsions are water-in-oil emulsions that comprise squalene and mannide mono-oleate (Arlacel™A), optionally with squalane, emulsified with the chimer protein in an aqueous phase. Well-known examples of such emulsions include Montanide™ ISA-720, and Montanide™ ISA 703 (Seppic, Castres, France), each of which is understood to contain both squalene and squalane, with squalene predominating in each, but to a lesser extent in Montanide™ ISA 703. Most preferably, Montanide™ ISA-720 is used, and a ratio of oil-to-water of 7:3 (w/w) is used. Other preferred oil-in-water emulsion adjuvants include those disclosed in WO 95/17210 and EP 0 399 843.

The use of small molecule adjuvants is also contemplated herein. One type of small molecule adjuvant useful herein is a 7-substituted-8-oxo- or 8-sulfo-guanosine derivative described in U.S. Pat. No. 4,539,205, No. 4,643,992, No. 5,011,828 and No. 5,093,318, whose disclosures are incorporated by reference. Of these materials, 7-allyl-8-oxoguanosine (loxoribine) is particularly preferred. That molecule has been shown to be particularly effective in inducing an antigen- (immunogen-) specific response.

A preferred useful adjuvant includes monophosphoryl lipid A (MPL), 3-deacyl monophosphoryl lipid A (3D-MPL), a well-known adjuvant manufactured by Ribi Immunochem, Hamilton, Mont. The adjuvant contains three components extracted from bacteria: monophosphoryl lipid (MPL) A, trehalose dimycolate (TDM) and cell wall skeleton (CWS) (MPL+TDM+CWS) in a 2% squalene/Tween 80 emulsion. This adjuvant can be prepared by the methods taught in GB 2122204B. A preferred form of 3-de-O-acylated monophosphoryl lipid A is in the form of an emulsion having a small particle size less than 0.2 μm in diameter (EP 0 689 454 B1). Most preferred are synthetic monosaccharide analogues of MPL called aminoalkyl glucosamide 4-phosphates (AGPs) such as sold under the designation RC-529 {2-[(R)-3-tetradecanoyloxy-tetradecanoyl-amino]-ethyl-2-deoxy-4-O-phosphono-3-O-[(R)-3-tetradecanoyl-oxytetradecanoyl]-2-[(R)-3-tetra-decanoyloxytetra-decanoylamino]-p-D-glucopyranoside triethylammonium salt}. RC-529 is available in a squalene emulsion sold as RC-529SE and in an aqueous formulation as RC-529AF available from Corixa Corp. (see U.S. Pat. No. 4,987,237 and No. 6,113,918). These adjuvants can be used alone or in combination with one or more other adjuvants such as Alhydrogel™.

Further contemplated adjuvants include synthetic oligonucleotide adjuvants containing the CpG nucleotide motif one or more times (plus flanking sequences) available from Coley Pharmaceutical Group. The adjuvant designated QS21, available from Aquila Biopharmaceuticals, Inc., is an immunologically active saponin fractions having adjuvant activity derived from the bark of the South American tree Quillaja Saponaria Molina (e.g. Quil™ A), and the method of its production is disclosed in U.S. Pat. No. 5,057,540; semi-synthetic and synthetic derivatives of Quillaja Saponaria Molina saponins are also useful, such as those described in U.S. Pat. No. 5,977,081 and No. 6,080,725. The adjuvant denominated MF59 available from Chiron Corp. is described in U.S. Pat. No. 5,709,879 and No. 6,086,901.

Muramyl dipeptide adjuvants are also contemplated and include N-acetyl-muramyl-L-threonyl-D-isoglutamine (thur-MDP), N-acetyl-nor-muramyl-L-alanyl-D-isoglutamine [CGP 11637, referred to as nor-MDP], and N-acetylmuramyl-L-alanyl-D-isoglutaminyl-L-alanine-2-(1′-2′-dipalmityol-sn-glycero-3-hydroxyphosphoryloxy)ethylamine [(CGP) 1983A, referred to as MTP-PE]. The so-called muramyl dipeptide analogues are described in U.S. Pat. No. 4,767,842.

Preferred adjuvant mixtures include combinations of 3D-MPL and QS21 (EP 0 671 948 B1), oil-in-water emulsions comprising 3D-MPL and QS21 (WO 95/17210, PCT/EP98/05714), 3D-MPL formulated with other carriers (EP 0 689 454 B1), QS21 formulated in cholesterol-containing liposomes (WO 96/33739), or immunostimulatory oligonucleotides (WO 96/02555). SBAS2 (now ASO2) available from SKB (now Glaxo-SmithKline) contains QS21 and MPL in an oil-in-water emulsion. Alternative adjuvants include those described in WO 99/52549 and non-particulate suspensions of polyoxyethylene ether (UK Patent Application No. 9807805.8).

Adjuvants are utilized in an adjuvant amount, which can vary with the adjuvant, mammal and recombinant HBc chimer immunogen. Typical amounts can vary from about 1 μg to about 1 mg per immunization. Those skilled in the art know that appropriate concentrations or amounts can be readily determined.

Inocula and vaccines are conventionally administered parenterally, by injection, for example, either subcutaneously or intramuscularly. Additional formulations that are suitable for other modes of administration include suppositories and, in some cases, oral formulation. The use of a nasal spray for inoculation is also contemplated as discussed in Neirynck et al. (1999) Nature Med., 5(10):1157-1163. For suppositories, traditional binders and carriers can include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate and the like.

An inoculum or vaccine composition takes the form of a solution, suspension, tablet, pill, capsule, sustained release formulation or powder, and contains an immunogenically effective amount of HBc chimer or HBc chimer conjugate, preferably as particles, as the active ingredient. In a typical composition, an immunogenically effective amount of preferred HBc chimer or HBc chimer conjugate particles is about 1 μg to about 1 mg of active ingredient per dose, and more preferably about 5 μg to about 50 μg per dose, as noted before.

A vaccine is typically formulated for parenteral administration. Exemplary immunizations are carried out sub-cutaneously (SC) intra-muscularly (IM), intravenously (IV), intraperitoneally (IP) or intra-dermally (ID). However, oral and nasal routes of vaccination are also contemplated.

The HBc chimer particles and HBc chimer particle conjugates can be formulated into the vaccine as neutral or salt forms. Pharmaceutically acceptable salts, include the acid addition salts (formed with the free amino groups of the protein or hapten) and are formed with inorganic acids such as, for example, hydrochloric or phosphoric acids, or such organic acids as acetic, oxalic, tartaric, mandelic, and the like. Salts formed with the free carboxyl groups can also be derived form inorganic bases such as, for example, sodium, potassium, ammonium, calcium, or ferric hydroxides, and such organic bases as isopropylamine, trimethylamine, 2-ethylamino ethanol, histidine, procaine, and the like.

In yet another embodiment, a vaccine or inoculum is contemplated in which a gene encoding a contemplated HBc chimer is transfected into suitably attenuated enteric bacteria such as S. typhi, S. typhimurium, S. typhimurium-E. coli hybrids or E. coli. Exemplary attenuated or avirulent S. typhi and S. typhimurium and S. typhimurium-E. coli hybrids are discussed in the citations provided before. These vaccines and inocula are particularly contemplated for use against diseases that infect or are transmitted via mucosa of the nose, the gut and reproductive tract such as influenza, yeasts such as Aspergillus and Candida, viruses such as polio, foot-and-mouth disease, hepatitis A, and bacteria such as Cholera, Salmonella and E. coli and where a mucosal IgA response is desired in addition to or instead of an IgG systemic response.

The enteric bacteria can be freeze dried, mixed with dry pharmaceutically acceptable diluents, made into tablets or capsules for ingestion and administered to or taken by the host animal as are usual solid phase medications. In addition, aqueous preparations of these bacterial vaccines are adapted for use in mucosal immunization as by oral, nasal, rectal or vaginal administration.

Oral immunization using plant matter containing contemplated chimeric molecule particles can be achieved by simple ingestion of the transgenic plant tissue such as a root, like a carrot, or seed, such as rice or corn. In this case, the water of the mouth or gastrointestinal tract provides the usually used aqueous medium used for immunization and the surrounding plant tissue provides the pharmaceutically acceptable diluent.

The inocula or vaccines are administered in a manner compatible with the dosage formulation, and in such amounts as are therapeutically effective and immunogenic. The quantity to be administered depends on the subject to be treated, capacity of the subject's immune system to synthesize antibodies, and degree of protection desired. Precise amounts of active ingredient required to be administered depend on the judgment of the practitioner and are peculiar to each individual. However, suitable dosage ranges are of the order of tens of micrograms active ingredient per individual. Suitable regimes for initial administration and booster shots are also variable, but are typified by an initial administration followed in intervals (weeks or months) by a subsequent injection or other administration.

Once immunized, the mammal is maintained for a period of time sufficient for the recombinant HBc chimer immunogen to induce the production of a sufficient titer of antibodies that bind to an antigen of interest such as a sporozoite for a malarial vaccine. The maintenance time for the production of illustrative anti-sporozoite antibodies typically lasts for a period of about three to about twelve weeks, and can include a booster, or second immunizing administration of the vaccine. A third immunization is also contemplated, if desired, at a time 24 weeks to five years after the first immunization. It is particularly contemplated that once a protective level titer of antibodies is attained, the vaccinated mammal is preferably maintained at or near that antibody titer by periodic booster immunizations administered at intervals of about 1 to about 5 years.

The production of anti-sporozoite, in the case of a malaria vaccine, or other antibodies is readily ascertained by obtaining a plasma or serum sample from the immunized mammal and assaying the antibodies therein for their ability to bind to an appropriate antigen such as a synthetic circumsporozoite immunodominant antigen [e.g. the P. falciparum CS protein peptide (NANP)₅ used herein] in an ELISA assay as described hereinafter or by another immunoassay such as a Western blot, as is well known in the art.

It is noted that the induced antibodies such as anti-CS antibodies or anti-influenza antibodies can be isolated from the blood of an inoculated host mammal using well known techniques, and then reconstituted into a second vaccine for passive immunization as is also well known. Similar techniques are used for gamma-globulin immunizations of humans. For example, antiserum from one or a number of immunized hosts can be precipitated in aqueous ammonium sulfate (typically at 40-50 percent of saturation), and the precipitated antibodies purified chromatographically as by use of affinity chromatography in which (NANP)₅ or an influenza M2 polypeptide is utilized as the antigen immobilized on the chromatographic column. Thus, for example, an inoculum can be used in a horse or sheep to induce antibody production against a malarial species for use in a passive immunization in yet another animal such as humans.

Another embodiment of the invention is a process for inducing antibodies, activated T cells or both in an animal host comprising the steps of inoculating said animal host with an inoculum. The inoculum used in the process comprises an immunogenic amount of a before-described HBc chimer particle or HBc chimer particle conjugate dissolved or dispersed in a pharmaceutically acceptable diluent. The animal host is maintained for a time sufficient for antibodies or activated T cells to be induced, as can be assayed by well-known techniques, which typically requires a time period of weeks to months, as is again well-known. A plurality of such immunizations is contemplated during this maintenance period.

### EXAMPLES

The following examples are given by way of illustration only and should not be considered limitations of this invention. Many variations on this technology are possible without departing from the spirit and scope of the invention. Immunological and recombinant DNA methods may not be explicitly described in this disclosure but are well within the scope of those skilled in the art. References cited here and elsewhere are explicitly included in their entirety.

### Example 1

### Construction of Stabilized HBc Polypeptide VLPs Using the M2 Extracellular Domain

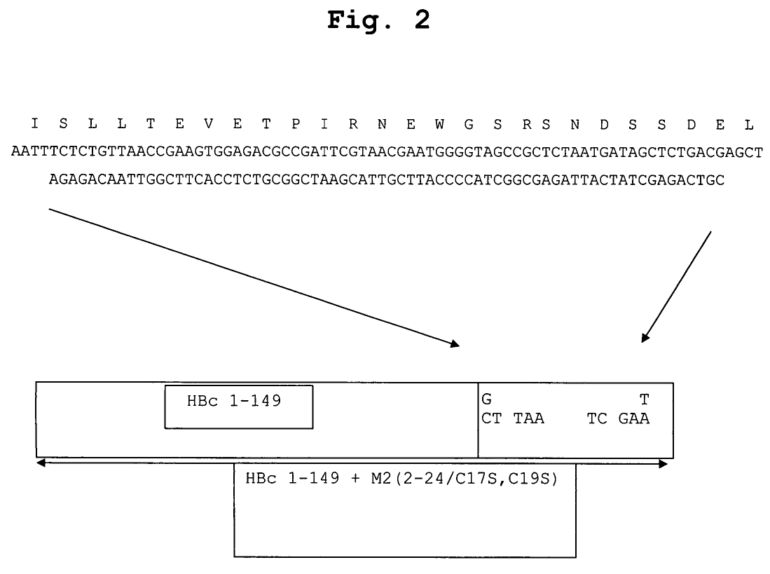

To illustrate the ability of the N-terminal region of the influenza virus M2 protein to stabilize hepatitis B virus core-like particles in a non-covalent manner, the extracellular domain of M2e was fused to the C-terminus of C-terminally truncated HBc polypeptides. To ensure that the intrinsic cysteine residues did not contribute to stabilization, a version of M2 (positions 2-24), where the cysteines were mutated to serines was used (CV-1895, V7.M2e(2C>2S). To construct the expression vector, V7.M2e(2C>2S), a pair of oligonulceotides was annealed and inserted into expression vector V7 (below), which accepts insertions after amino acid V149 of the HB capsid polypeptide gene. This is shown schematically in FIG. 2.

Thus, a new vector was constructed to enable the fusion of self-binding peptides to the C-terminus of a HBc chimer. Unique EcoRI and SacI restriction sites were inserted between HBc valine-149 and the HindIII site to facilitate directional insertion of synthetic dsDNAs into EcoRI-HindIII (or EcoRI-SacI) restriction sites. The pair of PCR primers below was used to amplify the HBc 149 gene with an NcoI restriction site at the amino-terminus and EcoRI, SacI and HindIII sites at the carboxyl-terminus. The product of the PCR reaction (479 bp) was digested with NcoI and HindIII and cloned into pKK223-3N to form vector V7.

To insert self-binding peptides, the plasmid (V7) was digested with EcoRI and HindIII (or EcoRI and SacI) and synthetic dsDNA fragments having EcoRI/HindIII (or EcoRI/SacI) over hangs, were ligated into V7. For all V7 constructs, the final amino acid of native HBc (valine-149) and the first amino acid of the inserted self-binding peptide are separated by a glycine-isoleucine dipeptide sequence coded for by the nucleotides that form the EcoRI restriction site. For epitopes inserted at EcoRI/SacI, there are additional glutamic acid-leucine residues after the self-binding peptide, prior to the termination codon, contributed by the SacI site. Restriction sites are again underlined in the primers shown.

HBc149/NcoI-F

SEQ ID NO:278

5′-TTGGGCCATGGACATCGACCCTTA
HBc149/SacI-EcoRI-H3 -R

SEQ ID NO:279

5′-CGCAAGCTTAGAGCTCTTGAATTCCAACAACAGTAGTCTCCG

The sequence of the M2 peptide C-terminal insert in single letter code, and it encoding DNA sequence for particle CV-1895 [M2(2-24/C17S,C19S)] are shown below.

| | |
|---|---|
| I S L L T E V E T P I R | SEQ ID NO:29 |
| AATTTCTCTGTTAACCGAAGTGGAGACGCCGATTCGT | SEQ ID NO:280 |
| AGAGACAATTGGCTTCACCTCTGCGGCTAAGCA | SEQ ID NO:281 |
| N E W G S R S N D S S D | |
| AACGAATGGGGTAGCCGCTCTAATGATAGCTCTGA | |
| TTGCTTACCCCATCGGCGAGATTACTATCGAGACT | |
| E L | |
| CGAGCT | |
| GC | |

### Example 2

### Analysis of Stabilized HB Capsid Polypeptide VLPs

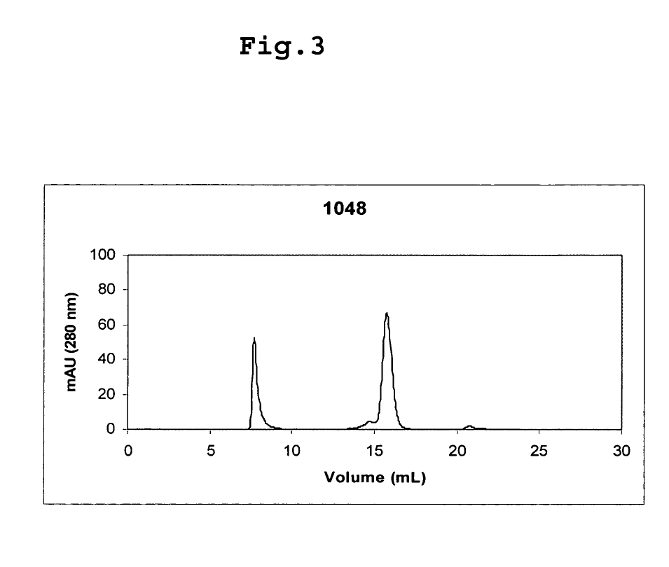

Following expression and purification, the particles were analyzed for their ability to retain their particulate morphology using analytical size exclusion chromatography. Particles without C-terminal stabilization (e.g. CV-1048) exist as a mixture of particulate and non-particulate material when analyzed in this manner (FIG. 3).

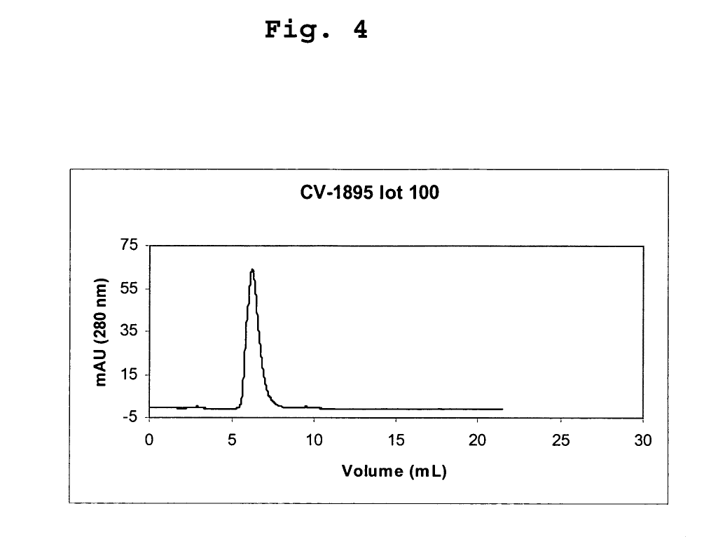

Similar analysis of CV-1895 particles revealed that they eluted as homogeneous particle structures (FIG. 4). These data indicate that the M2e(2-24, C17S, C19S) sequence, when present fused to the C-terminus of HBc, forms intermolecular interactions that stabilize the particle structure. Those data further indicate that the intermolecular stabilization occurs in the absence of the cysteine residues at M2 positions 17 and 19.

### Example 3

### Construction of Stabilized HBc Polypeptide VLPs Using the GCN4-p1 Leucine Zipper

To examine the ability of the leucine zipper domain of the yeast GCN4 transcriptional regulator to stabilize hepatitis B capsid polypeptide particles, in a non-covalent manner, a modified leucine zipper derived from GCN4 protein, GNC4-VL that forms both dimers and trimers in solution, is genetically fused to the C-terminus of C-terminally truncated HBc particles. Following the method of Example 1 to construct the expression vector, synthetic oligonulceotides encoding GCN4-VL are annealed and inserted into expression vector V7 that accepts insertions after amino acid V149 of the HB capsid polypeptide gene. The peptide attached to the HB capsid in this way has this sequence:

SEQ ID NO:282

RVKQLEDKVEELLSKVYHLENEVARLKKLVGER

Particles obtained are more stable by analysis using analytical size exclusion chromatography than the original CV-1048 VLPs and are substantially free of nucleic acid binding.

### Example 4

### Stabilized HBc VLPs with N-Terminal Lysine-Containing Linker Extensions

Following the teaching of Clarke et al., EP0385610, epitopes are attached to N-terminal lysine residues of suitably modified HBc polypeptide VLPs, for example CV-1895 is engineered to have an additional lysine residue at the HBc capsid polypeptide N-termini. More than one Lys residue can be present in the N-terminal extension. Such an engineered capsid polypeptide has a stabilizing peptide at the C-terminus, e.g., the extracellular self-assembly peptide of the M2 protein of influenza A, as in Example 1.

Here epitopes from the FMDV O1 and A12 subtypes are used. These are about amino acid residues 142 to about 160 of the VP1 capsid protein. These sequences can be repeated twice. For example, useful epitopes are:

FMDV O1

(FMDV O1 VP1 AA 137-162)-(FMDV O1 VP1 AA 137-162)-Cys;

in the one letter code, this sequence is

SEQ ID NO:283

NRNAVPNLRGDLQVLAQKVARTLPTSNRNAVPNLRGDLQVLAQKVARTLP
TSC;

FMDV A12,
(FMDV A12 VP1 AA 137-162)-(FMDV A12 VP1 AA 137-162)-Cys:

in the one letter code, this sequence is

SEQ ID NO:284

YSASGSGVRGDLGSLAPRVARQLPASYSASGSGVRGDLGSLAPRVARQLP
ASC

Shorter immunogenic sequences of the FMDV epitope can be presented, and can also be repeated.
Useful smaller peptides are:

(FMDV O1 VP1 AA 145-150)-(FMDV O1 VP1 AA 145-160)-Cys:

SEQ ID NO:285

RGDLQVRGDLQVLAQKVARTLPC;

and

(FMDV O1 VP1 AA 145-150)-(FMDV O1 VP1 AA 145-150)-(FMDV O1 VP1 AA 145-160)-Cys:

SEQ ID NO:286

RGDLQVRGDLQVRGDLQVLAQKVARTLPC.

The N-terminal linker extension can have any appropriate length provided the HBc with the extension can self-assemble into core-like particles and provided a Lys residue in the linker extension is exposed available for coupling. The linker extension can be up to 100 amino acid residues long or longer if desired. A short linker extension can be up to 60, for example up to 40, 20, 10 or 5, amino acid residues long.

One suitable extension comprises residues 95 to 102, for example 95 to 104, of the VP1 capsid protein of a strain of poliovirus type 1 (PV1) such as the Sabin or Mahoney strain:

Sabin PV1 VP1 95-102:

SEQ ID NO:287

SASTKNKD;
Sabin PV1 VP1 95-104:

SEQ ID NO:288

SASTKNKDKL
Mahoney PV1 VP1 95-102:

SEQ ID NO:289

PASTTNKD;
Mahoney PV1 VP1 95-104:

SEQ ID NO:290

PASTTNKDKL

As taught by Clarke EP0385610, in any N-terminal extension of HB capsid polyprotein, generally there should be at least one Lys residue within the first fourteen N-terminal residues. For a selected polypeptide used for coupling, it can be appropriate to choose a HBc polypeptide with a N-terminal extension comprising a Lys residue close to the N-terminus of the extension. Also, the N-terminal extension can be hydrophilic.

A modified HB capsid polypeptide with an N-terminal linker extension comprising one of these sequences or any other sequence incorporating a Lys residue can be obtained by standard genetic engineering techniques.

### Example 5

### Chemical Attachment of Heterologous Epitopes to Stabilized HBc VLPs Bearing N-Terminal Lysine-Containing Linker Extensions

Conjugates are prepared by coupling the epitope to the modified HBc VLP via the side chain amino group of the Lys residue present among the N-terminal linker extension amino acid residues of the modified HB capsid VLP. This coupling is typically achieved by reacting the modified HBc VLP with a bifunctional reagent capable of linking to an amino group and to a sulfhydryl group. The thus-derivatized modified HBc VLP is reacted with the epitope which, if not possessing a free sulfhydryl group, has been modified so that it does possess such a group.

Suitable bifunctional reagents include SMCC, MBS and N-succinimidyl-3-(2-pyridyldithio)-proprionate (SPDP). The bifunctional reagents often include a group that forms a peptide link and a group capable of forming a disulphide or thioether link.

A sulfhydryl group can be added to a peptide epitope by synthesis of the peptide to contain a cysteine residue, by reaction of an amino function with 2-iminothiolane or the N-hydroxysuccinimide ester of 3-(3-dithiopyridyl)-propionate or S-acetylthio-glycolic acid. After reaction with S-acetylthio-glycolic acid N-hydroxysuccinimide ester (SATA) deacetylation of the SATA with, for example, hydroxylamine produces free -SH groups. Small molecule epitopes, including drugs, are derivitized as is known in the chemical art.

The sulfhydryl-modified HBc VLPs are carboxamidomethylated before use to block any unreacted sulfhydryl groups on the VLPs after derivitization and so prevent VLPs from cross-linking to each other. Carboxamidomethylation is achieved by reacting the capsid polypeptide particles with iodoacetamide; excess reagent is removed by gel filtration or dialysis. Alternatively, and preferably, the sulfhydryl group is present on the added peptide as a cysteine mercaptan that is first reacted with the bifunctional cross-linking reagent. After purification, the so functionalized peptide is reacted with the N-terminal lysine of a HBc VLP. Similar chemistry is taught in U.S. Pat. No. 6,231,864 where a lysine residue was first added into the immunogenic loop of HBc.

In practice, the stabilized, linker-bearing HBc VLPs are generally provided in a buffer such as a phosphate buffer at a pH value of about 7. A molar excess of the bifunctional agent in an inert solvent, for example an aprotic organic solvent such as dimethylformamide, is added. Derivitization of the HB capsid VLPs occurs. Excess of the bifunctional agent is removed, by filtration for example. Excess of the epitope to be coupled to the HB capsid VLPs is then added. Coupling occurs and the resulting conjugates composed of the epitope linked to the modified HB capsid VLPs are recovered, for example by filtration. It is preferred to derivatize the peptide epitope first and react an excess of that derivatized peptide epitope with the stabilized VLP.

### Example 6

### Chemical Attachment of Self-Binding Peptides to the N- or C-Termini Of HB Capsid Polypeptides

Following the method of Clarke, EP0385610 and detailed in the previous example, HB capsid polypeptides without stabilizing peptides, e.g., derivatives of CV-1048 and others, bearing 1-6 additional lysines at the N- or C-termini, are chemically coupled to self-binding peptides. As in Example 1, the resulting VLPs bearing N- or C-coupled self-binding peptides are more stable using analytical size exclusion chromatography than the VLP comprised of unmodified CV-1048 or other HB capsid polypeptide.

### Example 7

### Coupling of the Peptide Composed of FMDV VP1 Residues 141-160, Plus a C-Terminal Cys Residue, to Stabilized, N-Terminal Linker Extension-Bearing HB Capsid VLPs

Stabilized HB capsid VLPs bearing a suitable N-terminal linker extension, e.g., as in Example 4 above, are purified on two consecutive sucrose gradients and passed at a concentration of 5 mg/ml in 10 mM phosphate buffer of pH 7.2 through a Sephadex® G100 column. These modified HB capsid polypeptide VLPs in 10 mM phosphate buffer at a concentration of 2 mg/ml are derivatized by adding 1/20 volume of SMCC (Pierce Biotechnology, Inc., Rockford, Ill.) dissolved in dry dimethylformamide so that the final concentration of SMCC in the VLP sample is a 50 times molar excess relative to the capsid polypeptide.

After 30 minutes at room temperature, the SMCC is removed by filtration through Sephadex® G100 in 10 mM phosphate buffer, pH 7.2. Freshly dissolved, in vitro-synthesized FMDV 141-160 Cys, having the sequence PNLRGDLQVLAQKVARTLPC (SEQ ID NO: 291), is added in 1/10 volume to provide a 10 times molar excess of the peptide with respect to the derivatized HBc polypeptide. After stirring at room temperature for 2 hours, uncoupled peptide is separated from the thus-derivatized VLPs by filtration through Sephadex® G200. The stabilized HB capsid VLPs with the peptide attached are recovered. These VLPs are more stable than identically processed VLPs based on CV-1048 lacking the self-binding peptide as determined by analytical gel filtration chromatography.

### Example 8

### Coupling of HRV2 Peptide to Stabilized HB Capsid VLPs

Following the procedure described in Example 7, the HRV2 peptide that encompasses the NIm-II epitope and has the sequence:

| | |
|---|---|
| VKAETRLNPDLQPTC | SEQ ID NO: 292 |

was coupled to stabilized HB capsid polypeptide VLPs bearing N-terminal linker extensions. VLPs with peptide attached were recovered. These VLPs are more stable when assayed using analytical size exclusion chromatography than otherwise identical VLPs lacking the self-binding peptide, as discussed previously.

### Example 9

### Coupling of HBsAg B-Cell Epitope to Stabilized HB Capsid VLPs Bearing N-Terminal Linker Extensions

Stabilized HB capsid VLPs bearing N-terminal linker extensions from Example 4 were derivatized with an equimolar amount of SMCC in DMF as in Example 7. Freshly synthesized peptides comprising the HBsAg B-cell epitope from the preS protein, residues 132-145, plus an extra C-terminal Cys, having the sequence

| | |
|---|---|
| QDPRVRGLYFPAGGC | SEQ ID NO: 293 |

are reacted with the freshly SMCC-derivatized stabilized HB capsid VLPs bearing N-terminal linker extensions by the method of Example 7. VLPs including self-binding peptides such as leucine-zipper peptides, are more stable than otherwise identical VLPs lacking the self-binding peptide when assayed using analytical size exclusion chromatography.

### Example 10

### Stabilized VLPs Derived from the Capsid Polypeptide of Bacteriophage MS2

Bacteriophage MS2 is a bacteria-infecting member of the virus family Leviviridae. A plasmid expression vector for the coat protein of RNA bacteriophage MS2 is modified to introduce a unique KpnI restriction site within the coat protein gene at a site corresponding between residues 15 and 16, as taught by Mastico (1993) J. Gen. Virol., 74: 541-548 and Brown (2002) Intervirology, 45:371-380. Insertion of peptide-encoding DNA oligonucleotides at this site permits the production of chimeric MS2 coat proteins having foreign peptide sequences expressed as the central part of the exterior-located hairpin. A unique restriction site is also engineered at the 3′ end, encoding the C-terminus of the capsid polypeptide. Insertion of peptide-encoding DNA oligonucleotides at this site permits the production of chimeric MS2 coat proteins bearing C-terminal self-binding peptides independent of any inserted heterologous epitopes.

In this Example, oligonucleotides coding for an epitope from the L1 protein of human papilloma virus 16, corresponding to the protein the sequence

| | |
|---|---|
| PNDTFIVSTNPNTVTSSTPI | SEQ ID NO: 294 |

are inserted into KpnI site in the MS2 capsid polypeptide coding sequence.

Epitope-containing capsid polypeptides lacking the self-binding peptide self-assemble into substantially RNA-free VLPs in Escherichia coli that can be easily disassembled and reassembled in vitro after extraction.

Oligonucleotides encoding the wild-type leucine zipper peptide of the GCN4 protein (GCN4-p1) having the sequence

| | |
|---|---|
| RMKQLEDKVEELLSKNYHLENEVARLKKLVGER | SEQ ID NO: 295 |

are inserted into the site engineered at the 3′ end of the capsid polypeptide coding sequence. In this way the C-terminus of the epitope-containing capsid polypeptide is fused to the GCN4-p1 peptide upon expression in E. coli. The resulting VLPs are highly immunogenic and are more stable than the VLPs consisting of the same fusion polypeptide lacking the self-binding peptide when assayed using analytical size exclusion chromatography.

### Example 11

### Stabilized VLPs Derived from Human Polyomavirus JC VP1 Expressed in Saccharomyces cerevisea

Human polyomavirus JCV capsid polypeptide (VP1) lacking the first 12 amino acids does not encapsidate host DNA although the integrity of the capsid-like structure is maintained. [Ou et al. (2001) J. Med. Virol. 64:366-73] The 12 amino-terminal and 16 carboxy-terminal amino acids of VP1 are dispensable for the formation of virus-like particles, and further truncation at either end of VP1 leads to the loss of this property. [Ou et al. (2001) J. Neurovirol. 7:298-301]. The N-terminal 12 amino acids are replaced by the GCN4-VL peptide, which forms trimers and dimers in solution as an N-acetylated peptide; the sequence of the GCN4-VL leucine zipper peptide is

| | |
|---|---|
| RVKQLEDKVEELLSKVYHLENEVARLKKLVGER | SEQ ID NO: 296 |

The replacement is made by inserting hybridized, complimentary oligonucleotides encoding the peptide into the capsid polypeptide-encoding plasmid instead of merely recircularizing the plasmid after removal of the DNA segment encoding the N-terminal 12 amino acids. When expressed in yeast from the strongly inducible pGAL1 promoter, the empty VLPs formed from chimeric capsid polypeptide are more stable as determined using analytical size exclusion chromatography than the otherwise identical VLPs lacking the self-binding peptide.

### Example 12

### Stabilized Flock House Virus VLPs Bearing Heterologous Epitopes

Following the method of Hall, U.S. Pat. No. 6,171,591, epitopes were inserted into the exterior loop of the nodavirus Flock House virus capsid protein between residues 205-209. Corresponding loops are also found on the surface of each of the other family Nodaviridae members such as Black Beetle virus and Nodamura virus. Analogous insertions are easily made because of the structural similarities among this group of viruses. The N-terminal 20 residues are deleted to disable nucleic acid binding; synthetic oligonulceotides encoding the GCN4-VL peptide are annealed and inserted into expression vector to replace the capsid polypeptide N-termini. Expressed VLPs are purified from a baculovirus expression system, and are more stable to upon analysis using analytical size exclusion chromatography than are otherwise similar VLPs that do not contain a self-binding peptide sequence.

### Example 13

### From the Nudaurelia capensis Omega Virus Capsid Polypeptide Analogue-Based Stabilized VLPs

Loops corresponding to the epitope insertion site of Flock House virus can also be found on the surface of the related Tetraviridae, and the tetravirus Nudaurelia capensis omega virus was accordingly modified in a manner analogous to that in Example 13. The epitope is inserted between amino acid residues 380 and 381 in the exterior loop that extends from residue number 374 to residue 382 in the Ig-like domain of the capsid polypeptide. [Munshi et al., (1996) J. Mol. Biol. 261:1-10] The tetramer-forming peptide from tetrabrachion [Stetefeld (2000) Nat. Struc. Biol. 7:772-776] replaces the N-terminal 44 residues absent from Xtal structure to stabilize the VLP and reduce nucleic acid binding. The particles obtained from the baculovirus expression system are more stable than empty capsids lacking the tetrabrachion tetramer-forming peptide when the expressed VLPs are assayed using analytical size exclusion chromatography.

### Example 14

### Stabilized VLPs Derived from the Brome Mosaic Virus Capsid Polypeptide

Brome mosaic virus is a plant-infecting virus that is the type member of the Bromoviridae. Replacement of the N-terminal 25 residues abrogates nucleic acid binding but also leads to reduced VLP stability when the deleted BMV capsid polypeptide is expressed in yeast as taught in U.S. Pat. No. 5,869,287 to Price et al. Stability, but not nucleic acid binding, is restored by replacing the deleted residues with the human AP-4 protein leucine zipper peptide, having the sequence

IFSLEQEKTRLLQQNTQLKRFIQEL (SEQ ID NO:297).

### Example 15

### Stabilized VLPs Derived from the Turnip Crinkle Virus Capsid Polypeptide

Turnip crinkle virus (TCV) is a member of the [comoviruses]. The 40 N-terminal residues of the capsid polypeptide have nucleic acid binding ability, and are either deleted or replaced with the GCN-4 p1 self-binding leucine zipper peptide. When expressed in Saccharomyces cerevisea under the control of the strongly inducible GAL1 promoter, VLPs can be extracted by vortexing with glass beads in VSB buffer [0.1M NaCl, 0.1M NaOAc, 10 mM EDTA, pH 5.0]. Stability of TCV-derived VLPs lacking the nucleic acid binding domain is compared to that of VLPs bearing the self-binding peptide by incubation in 50 mM phosphate buffer at pH 7.2. Analytical size exclusion chromatography, according to Example 2, demonstrates that the VLPs with the self-binding peptide replacement of the N-terminal domain are more stable than those with the deletion alone.

### Example 16

### Stabilized VLPs Derived Human Papilloma Virus 16 L1 Capsid

Human papillomavirus 16 is a member of the Papillomaviridae. The self-binding leucine zipper peptide GCN4-VL RVKQLEDKVEELLSKVYHLENEVARLKKLVGER (SEQ ID NO: 298) is genetically fused to the N-terminus of HPV 16 L1. An epitope, e.g., the HPV 16 L2 sequence LVEETSFIDAGAP (SEQ ID NO:299) is inserted into the immunodominant external loop at residues 351-355. This context has been shown to enhance the immunogenicity of a foreign peptide when present in an assembled VLP. [Slupetzky et al., (2001) J. Gen. Virol. 82:2799-804] Baculovirus expression is used because Kirnbauer, (1992) Proc. Natl. Acad. Sci. 89:12180-4 found this system to be optimal. The VLPs bearing the N-terminal leucine zipper peptide are significantly more stable and hence more immunogenic than identical VLPs lacking the self-binding peptide.

### Example 17

### Stabilized VLPs Derived from the Norwalk Virus Capsid Polypeptide

Norwalk virus is a member of the Caliciviridae family of viruses. VLPs derived from the Norwalk virus capsid polypeptide are produced using the baculovirus expression system. When so produced, these VLPs assemble into particles lacking RNA. Because they are dispensable for particle formation, residues 228-530 (the entire exterior P domain) are deleted from the sequence and the N-terminal 20 residues are replaced by a dimerizing leucine zipper to stabilize interaction between B and C subunits. Resulting particles lacking the P domain are more stable than VLPs otherwise identical, but lacking the self-binding peptide.

### Example 18

### Stabilized VLPs Derived from the VP2 Human B19 Parvovirus Major Capsid

The human B19 parvovirus is a member of the Parvoviridae. Empty VLPs derived from the B19 VP2 major capsid polypeptide are produced by expression of VP2 in insect cells from a baculovirus expression vector under the control of the polyhedron promoter. [Kajigaya et al., (1991) Proc. Nat. Acad. Sci. 88:4646-4650] The first 25 amino acid residues are deleted from the N-terminus without disruption of VLP self-assembly. [Kawase et al., (1995) Proc. Natl. Acad. Sri. 69:6567-6571] A derivative of the N-terminal deletion VP2 is made where the N-terminal 25 residues of VP2 are replaced with the c-Myc leucine zipper self-binding peptide YLSVQAEEQKLISEEDLLRKRREQLKHKLEQL (SEQ ID NO:300). Both the simple deletion VP2 polypeptide and the replacement derivative are extracted from insect cells following expression from a baculovirus vector. VLPs comprising the chimeric VP2 polypeptide with the leucine zipper are more stable than the VLPs comprising the simple deletion VP2 polypeptide.

### Example 19

### Stabilized VLPs Derived from the Sindbis Virus Capsid

Sindbis virus is a member of the Togaviridae. The N-terminal 109 amino acid residues of the capsid polypeptide are implicated in inter-capsid polypeptide interactions required for virion assembly as well as nucleic acid binding. [Lin et al., (2000) J. Virol. 74:493-504] To abrogate nucleic acid binding and stabilize the VLP assembled from the chimeric polypeptide, the N-terminal 109 amino acids of the capsid polypeptide are replaced by tandem leucine zipper self-binding peptides. In this construct, two self-binding peptides are inserted: the GCN4-p1 leucine zipper peptide is N-terminal followed by a flexible linker segment of five glycines, followed by the c-Myc leucine zipper self-binding peptide. When expressed in BHK cells following the method of Frolov [(1997) J. Virol. 71:2819-2829], Sindbis capsid polypeptides bearing the double-leucine zipper as a replacement for the N-terminal 109 amino acids of the capsid polypeptide VLPs, whereas the corresponding N-terminal deletion capsid polypeptide is not competent for assembly.

### Example 20

### Stabilized VLPs Derived from the Physalis Mottle Virus Capsid

Physalis mottle virus is a member of the tymovirus genus. When the capsid polypeptide bearing a deletion of the N-terminal 30 amino acid residues is expressed in E. coli following the method of Sastri [(1997) J. Mol. Biol. 272:541-52], VLPs are readily recoverable. VLPs are also readily obtained from E. coli expressing capsid polypeptide derivatives in which the N-terminal 30 amino acid residues are replaced by the GCN4-VL leucine zipper. When extracted from E. coli, these VLPs are much stable at neutral pH values than are those lacking the self-binding peptide.

### Example 21

### Stabilized VLPs Derived from Rotavirus Capsid Polypeptides

Rotavirus is a member of the Reoviridae. VLPs bearing large replacements of the N-terminal 92 amino acid residues of VP2 can be expressed using a vaccinia vector in mammalian cell. Such VLPs exhibit reduced nucleic acid binding. [Charpilliene (2001) J. Biol. Chem. 276:29361-29367] To stabilize rotavirus VLPs, COMP pentamerization domain is genetically fused to the N-terminus of VP2 inasmuch as large N-terminal fusions are acceptable. When extracted, VLPs comprising VP2 with residues 1-92 replaced by the COMP self-binding peptide are more stable than those comprising VP2 with residues 1-92 deleted.

### Example 22

### Stabilized VLPs Derived from the Tobacco Mosaic Virus (TMV) Capsid

Tobacco mosaic virus (TMV) is the type member of the tobamovirus genus. In the absence of an RNA bearing the TMV genomic packaging signal, TMV capsid polypeptide monomers in solution can self-assemble into double-layered hollow-centered disks, each disk having approximately 17 copies of the capsid polypeptide. Amino acid residues 89-114 form the interior loop that is exposed in the hollow core of both these disks and the virion. The sequence of these residues is

D TRNRIIEVENQ QSPTTAETLD ATRR (SEQ ID NO:301)

To stabilize RNA-free disks, stabilizing elements are placed so they interact within the hollow core of the TMV CP disk. Thus, the capsid polypeptide (CP) of TMV strain L is engineered to change the lysines at positions 54 and 69 to glycines, and the glutamine at position 100 (underlined above) is changed to lysine to act as a linker site. In this way, only there is only one lysine in the CP, in the interior loop.

Metal-ion assisted self-binding peptides such as those of SEQ ID NOs: 1 and 2 in carboxyl form are chemically coupled to the interior loop lysine linker of the engineered TMV CP such that the resulting linked, engineered TMV CP bears a metal-ion assisted self-binding peptide. The derivatized TMV CP self assembles in the presence of appropriate metal ions into double layered disks. The stabilized double disk is substantially more stable to changes in temperature and ionic strength than are disks formed from the wild-type TMV CP as measured by analytical HPLC and centrifugation.

If desired, a heterologous epitope can be genetically engineered to be present as a C-terminal extension of the engineered CP. Because the C terminus is disordered in the crystal structure and present on the exterior surface of assembled CP, the structure of the epitope can be independent of the TMV CP carrier.

Full TMV L CP Sequence

| | |
|---|---|
| 1 msysitspsq fvflssvwad piellnvctn | SEQ ID NO: 302 |
| 31 slgnqfqtqq arttvqqqfs evwkpfpqst | |
| 61 vrfpgdvykv yrynavldpl itallgafdt | |
| 91 rnriievenq qspttaetld atrrvddatv | |
| 121 airsainnlv nelvrgtgly nqntfesmsg | |
| 151 lvwtsapas | |

### Example 23

### Stabilized VLPs Derived from the Cowpea Mosaic Virus Capsid

Cowpea mosaic virus is the type member of the Comoviridae. The S capsid polypeptide of Cowpea mosaic virus has been shown to tolerate insertions of heterologous peptides between positions 22 and 23. [Porta et al. (1994) Virology 202: 949-955] For this Example, an epitope from the Sabin strain of poliovirus, corresponding to VP1 residues 95-104 with the sequence SASTKNKDKL (SEQ ID NO: 303) is inserted at that site by replacing the Nhe I/Aat II fragment of pMT7-FMDV-II with annealed complimentary oligonucleotides encoding the epitope and residues 18-22 and 23-26 of the S polypeptide that would have otherwise been lost. When both the CPMV S and L are expressed in insect cells, substantially RNA-free VLPs are obtained. [Shanks (2000) J. Gen. Virol. 81:3093-3097] Fusion of the COMP peptide, having the sequence LKFRFRDIERSKRSVMVGHTATAA (SEQ ID NO: 304) to the N-terminus of the S polypeptide results in VLPs that are more stable than are particles comprised of capsid polypeptides lacking the self-binding peptide.

### Example 24

### Stabilized VLPs Derived from the Yeast Ty1 Transposon Capsid

The yeast Ty1 transposon is a member of the Pseudoviridae. Following the method of Kingsman, U.S. Pat. No. 5,463,024, the TyA capsid polypeptide of Ty1 is modified so the metal-ion assisted, trimerizing self-binding peptide with the sequence GELAEQKLEQALQKLA (SEQ ID NO:305) is attached by genetic fusion to the carboxy terminus of the capsid polypeptide. A minimal particle forming TyA polypeptide, corresponding to residues 286 to 381 is used. VLPs are produced in a Ty1-free strain of yeast, e.g., S. cerevisiae MD40-4c (urd2, trp1, leu2-3, leu2-112, his3-11, his3-15).

Ty-VLPs are purified as follows: Yeast cells are grown selectively at 30 degrees C. to a density of about 8×10⁶ cells/ml. The cells are then collected by low speed centrifugation, washed once in ice-cold water and resuspended in TEN buffer (10 mM Tris, pH 7.4; 2 mM EDTA; 140 mM Nacl) at 1 ml per 1 liter of cells. The cells are disrupted by vortexing with glass beads (40 mesh) at 4 degrees C. until more than 70 percent are broken. The beads are pelleted by low speed centrifugation, then the supernatant is collected and the debris removed by centrifugation in a microfuge for 20 minutes. The Ty-VLPs are then pelleted from the supernatant by centrifugation at 100,000 g for 1 hour at 4 degrees C. and are resuspended overnight (about 18 hours) in TEN buffer. The resuspended Ty-VLPs are centrifuged in a microfuge for 15 minutes at 4 degrees C. to remove cell debris prior to loading the supernatant onto a 15-45 percent (w/v) sucrose gradient in 10 mM Tris, pH 7.4; 10 nM NaCl and spinning at 76,300 g for 3 hours at 15 degrees C. Fractions are collected through the bottom of the tube and the peak fractions identified by Coomassie blue staining of SDS-PAGE gels on which aliquots of the fractions are electrophoresed. VLPs are concentrated by centrifugation of the peak fractions at 100,000 g for 1 hour at 4 degrees C.

Following incubation with an appropriate metal ion, e.g., Ru(III), VLPs bearing the self-binding peptide are more stable than those lacking the self-binding peptide.

### Example 25

### Stabilized VLPs Derived from the Hepatitis C Virus Capsid

The Hepatitis C virus is a member of the Flaviviridae. When expressed in insect cells according to the method of Baumbert et al., [(1998) J. Virol. 72:3827-3836], the capsid polypeptide forms VLPs. To the C terminus of the flavivirus capsid polypeptide is added the trimer-forming self-binding leucine zipper peptide GCN4-LL, which has the sequence

| | |
|---|---|
| RLKQLEDKLEELLSKLYHLENELARLKKLVGER | SEQ ID NO: 306 |

Following expression of VLPs comprising the capsid protein plus the self-binding peptide, it is found that the chimeric VLPs are more stable in phosphate-buffered saline (0.9 percent NaCl, 50 mM NaHPO₄ pH 7.3) than are VLPs comprising the unmodified capsid polypeptide.

### Example 26

### Stabilized VLPs Derived from the Capsid Polypeptide of Poliovirus

The polio virus is a member of the Picornaviridae. Empty capsids are a natural product of infections by most of the Picornaviridae. Empty capsids with native antigenicity are formed in S. cerevisea only if the capsid precursor P1 and the viral protease are co-expressed and purified in the presence of the capsid stabilizing compound pirodavir. [Rombaut (1997) J. Gen. Virol. 78:1829-1832] To the C-terminus of P1 (which is subsequently cleaved by the 3CD protease in vivo to become VP1 thru 4) is added the metal-ion assisted self-binding peptide preceded by five glycines to act as a flexible linker; the resulting C-terminal addition has the sequence GGGGGGLAQKLLEALQKALA (SEQ ID NO: 307). Following expression of the chimeric polypeptide in and purification from yeast in the presence of pirodavir, empty poliovirus capsids bearing the metal-ion assisted self-binding peptide are obtained. Following incubation with an appropriate metal ion, e.g. Ru⁺³, the stability of these capsids increases significantly relative to either the same VLPs without the metal ion or VLPs comprising poliovirus capsid polypeptides without the self-binding peptide.

### Example 27

### Stabilized VLPs Derived from the Measles Virus Capsid

The measles virus is a member of the Paramyxoviridae. Following the method of Warnes [Gene (1995) 160:173-178] the measles virus (MV) gene encoding the nucleocapsid (capsid) polypeptide is expressed in E. coli. Expression of the intact N gene under the control of the tac promoter in the pTrc99c plasmid produces a protein of the correct size (60 kDa) that represents approximately 4 percent of the total cellular protein, and is recognized by known measles positive human sera. “Herringbone” structures, characteristic of paramyxovirus nucleocapsids, are readily identified in fractured cells examined by electron microscopy. The gene encoding the metal-ion assisted self-binding peptide, the metal-ion assisted self-binding peptide sequence GGGGGGLAQKLLEALQKALA (SEQ ID NO: 308), preceded by a nucleic acid sequence encoding five glycines to act as a flexible linker is linked in frame to the C-terminus of the N gene. Following expression of the chimeric polypeptide in and purification from E. coli, measles virus capsids bearing the metal-ion assisted self-binding peptide are obtained. Following incubation with an appropriate metal ion, e.g. Ru⁺³, the stability of these capsids increases significantly relative to either the same VLPs without the metal ion or VLPs comprising measles capsid polypeptides without the self-binding peptide.

### Example 28

### Stabilized VLPs Derived from the Influenza A Virus Capsid

The influenza viruses are members of the Orthomyxoviridae. The N-terminal 189 amino acid residues are implicated in RNA binding. VLPs are obtained from insect cells using the baculovirus expression system to produce an influenza A nucleocapsid polypeptide from which the N-terminal RNA-binding domain has been deleted. To stabilize the VLPs, the N-terminal RNA-binding domain is replaced by the GCN4-p1 leucine zipper self-binding peptide discussed previously. When VLPs bearing the self-binding peptide are expressed in

Exemplary Epitopes and Linkers Used In
Chimeric Capsid Protein Constructs

| | |
|---|---|
| Epitope | Reference |

| | |
|---|---|
| residues 141-160 of VP1 of | U.S. Pat. No. 5,874,087 |

FMDV strain O.sub.1
VPNLRGDLQVLAQKVARTLP (SEQ ID NO:309)
FROM U.S. Pat. 6171591
B cell epitope
Vesicular Stomatitis virus (VSV)
Kreis (1986) EMBO J. 5:931-941
G glycoprotein
Kolodziej (1991) Meth. Enz. 194:508-519
Viral strain: ts-045 VSV Indiana serotype
YTDIEMNRLGK (SEQ ID NO:310)
Contiguous B and T cell epitopes
Bovine Respiratory Syncytial virus (BRSV)
Walravens et. al. (1990) J. Gen Virol. 71:3009-3014
F protein
Bourgeois et. al. (1991) J. Gen. Virol. 72:1051-1058
Viral strain: RB94
DKELLPKVNNHDCQISNIATVIEFQQ (SEQ ID NO:311)
Contiguous B and T cell epitopes
Human Respiratory Syncytial virus (RSV) F protein
Viral strain: RSS-2 (subtype A)
DKQLLPIVNKQSCSISNIETVIEFQQ (SEQ ID NO:312)
Contiguous B and T cell epitopes
Human Respiratory Syncytial virus (RSV) F protein
Viral strain: 18537 (subtype B)
DKRLLPIVNQQSCRINSNIETVIEFQQ (SEQ ID NO:313)
T cell epitope
Human Respiratory Syncytial virus (RSV) &
Bovine Respiratory Syncytial virus (BRSV)
F protein
Viral strains: RSS-2 (subtype A) (RSV),
18537 (subtype B) (RSV) &
RSS-2 (subtype A) (BRSV)
FPSDEF [100 percent sequence conservation] (SEQ ID NO:314)
B cell epitope
Hepatitis B Virus (HBV)
Neurath et al. (1986) Vaccine 4:35
preS2 Residues 132-145
Itoh et al (1986) Proc. Natl. Acad. Sci. 83:9174.
QDPRVRGLYFPAGG (SEQ ID NO:315) *double chimera made
with epitope below
overlapping Th and CTL epitopes
Hepatitis B Virus (HBV)
Francoet al. (1997) J. Immunol. 159:2001-2008.
HBsAg residues 178-204
Greenstein et al. (1992) J. Immunol. 148:3970.
LQAGFFLLTRILTIPQSLDSWWTSLNF (SEQ ID NO:316)
From U.S. Pat. 6,174,528 to Cooper et al.
Streptococcal group A epitope
Streoptococcal M protein p145 amino acids 337-356
LRRDLDASREAKKQVEKALE (SEQ ID NO:317)
From U.S. Pat. No. 6,060,064
V3 loop from HIV-1 Isolates:

| | | |
|---|---|---|
| BH10 | SNCTRPNNNTRKSIRIQRGPGRAFVTIGKIGNMRQAHCNISG | (SEQ ID NO:318) |
| HXBII | SNCTRPNNNTRKTRKRIRIQRGPGRAFVTIGKIGNMRQAHCNISG | (SEQ ID NO:319) |
| MN | SNCTRPNYNKRKRIHIGPGRAFYTTKNIIGTIRQAHCNISG | (SEQ ID NO:320) |
| MAL | SNCTRPGNNTRRGIHFGPGQALYTTGIVDIRRAYCTING | (SEQ ID NO:321) |
| RF | SNCTRPNNNTRKSITKGPGRVIYATGQIIGDIRAHCNLSGS | (SEQ ID NO:322) |
| ELI | STCARPYQNTRQRTPIGLGQSLYTTRSRSIIGQAHCNISG | (SEQ ID NO:323) |
| MAL (var) | SNCTRPGNNTRRGIHFGPGQALYTTGIVDEIRRAYCNISG | (SEQ ID NO:324) |
| RF (var) | SNCTRPNNTRKSITKQRGPGRVLYATGQIIGDIRKAHCNSIG | (SEQ ID NO:325) |
| ELI (var) | STCARPYQNTRQRTPIGLGQSLYTTRGRTKIIGQAHCNISG | (SEQ ID NO:326) |

From U.S. Pat. No. 6,110,466
Amino acids 735-752 of HIV-1 gp41
Dagleish, A.G et al., (1988) Virology 165:209-215;
Chahn, T.C. et al. (1986) EMBO J. 5:3065-3071.
DRPEGIEEEGGERDRDRSD (SEQ ID NO:327)
Amino acids 85-99 from VP1 of human rhinovirus 14
U.S. Pat. No. 6,110,466 to Lomonossoff et al.
PATGIDNGREAKLD (SEQ ID NO:328)
T cell epitope
Plasmodium falciparum LSA-1 T1 epitope
Heal et al., (200) Vaccine 18:251-258
LTMSNVKNVSQTNFKSLLRNLGVS (SEQ ID NO:329)
Insertions in M2 btw G 14 and T 15 fo cp.
Mastico et al., 1993 Intervirology 74:541-548
HA9 YPYDVPDYA (SEQ ID NO:330)
Wilson et al. (1984) Cell 37:767-778
IgE FVFFGSKTK (SEQ ID NO:331)
Stanworth et al., (1990) Lancet 336:1279-1281
Mal (Surf. Ag) NANPNANPNANP (SEQ ID NO:332)
Greenwood et al., (1991) J. Mol. Biol. 220:821-827
HPV L1 PNDTFIVSTNPNTVTSSTPI (SEQ ID NO:334)
Javaherian at al., (1989) Proc. Natl. Acad. Sci.
86:6768-6772
HIV gp120 NNTRKSIRIQRGPGRAFVTIGKIG (SEQ ID NO:335)
B and T cell epitope
Pseudomonas aeruginosa PAK pilin amino acids 82-104
Smartet al., (1988) Infec. Immun. 56:18-23
GTIALKPDPADGTADITLTFTM (SEQ ID NO:336)

Other Insertions:

Biotin-binding peptides (from U.S. Pat. No. 6,380,364)

| | |
|---|---|
| GGGCSWAPPFKASC | (SEQ ID NO: 337) |
| GGGRGEFTGTYITAVT | (SEQ ID NO: 338) |

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed is:

1. A chimeric polypeptide comprising:
a) a first polypeptide portion that self-assembles into organized, repetitive, supramolecular structures in aqueous solution having at least about 9 subunits, and
b) a second peptide portion that comprises a peptide having a length of about 15 to about 80 amino acid residues, said peptide
i) when present as an N-acetylated peptide self-assembling to form parallel multimers in aqueous PBS solution at pH 7.0 and a concentration of about 10 millimoles/liter,
ii) self assembling when present in solution at a concentration of at least ten micromoles/liter in the presence of a five-fold molar excess of a predetermined multivalent metal ion, or
iii) is the self-assembling extracellular domain of the influenza A M2 polypeptide, with the proviso that the second portion is other than that extracellular M2 domain, which contains zero, one or two cysteines, N-terminally bonded to a first portion that is a HBc polypeptide;
said first and said second portions being linked by a covalent bond, and said chimeric polypeptide forming organized, repetitive, supramolecular structures that are more stable than are structures formed from said first portion alone.

2. The chimeric polypeptide according to claim 1 where said first and said second portions are linked by a peptide bond.

3. The chimeric polypeptide according to claim 1 where said first portion includes a linker residue.

4. The chimeric polypeptide according to claim 3 where an epitope is attached to said linker residue.

5. The chimeric polypeptide according to claim 1 where said first portion includes a heterologous epitope.

6. The chimeric polypeptide according to claim 5 that elicits an immune response when immunized into a host animal.

7. The chimeric polypeptide according to claim 1 where said first portion comprises the E2 polypeptide of the pyruvate dehydrogenase enzyme complex.

8. The chimeric polypeptide according to claim 1 where said first portion comprises the lumazine synthase polypeptide.

9. The chimeric polypeptide according to claim 1 where said first portion is a virus capsid polypeptide or an analogue of a virus capsid polypeptide.

10. The chimeric polypeptide according to claim 9 where said first portion comprises the capsid polypeptide or an analogue of a virus capsid polypeptide of an animal-infecting virus, a plant-infecting virus, a bacteria-infecting virus or a fungus-infecting virus.

11. The chimeric polypeptide according to claim 1 wherein said first portion comprises the N-terminal portion of the polypeptide and said second portion comprises the C-terminal portion of said polypeptide.

12. The chimeric polypeptide according to claim 10 where said first portion comprises the capsid polypeptide of the hepatitis B virus.

13. The chimeric polypeptide according to claim 1 wherein the organized, repetitive, supramolecular structures are virus-like particles (VLPs).

14. The polypeptide according to claim 1 where said first portion comprises a polypeptide from the capsid of a viruses selected from the group consisting of the families Picornaviridae, Caliciviridae, Togaviridae, Flaviviridae, Rhabdoviridae, Paramyxoviridae, Orthomyxoviridae, Reoviridae, Retroviridae, Polyomaviridae, Papillomaviridae, Parpovaviridae, Hepadnaviridae, Nodaviridae, Tetraviridae, Tombusviridae, Comoviridae, Geminiviridae, Bromoviridea, Potyviridea, Inoviridae, Leviviridae, Microviridae, Pseudoviridae, Totiviridae, Metaviridae, and Tymoviridae and the virus genera tobamovirus, tymovirus, potexvirus, and sobemovirus.

15. The chimeric polypeptide according to claim 1 where said first portion comprises a self-assembling polypeptide subunit of an icosahedral enzyme complex.

16. A recombinant chimer hepatitis B core (HBc) protein molecule up to about 570 amino acid residues in length that
(a) contains a sequence of at least about 135 of the N-terminal 150 amino acid residues of the HBc molecule that include a peptide-bonded heterologous epitope or a heterologous linker residue for a conjugated epitope present in the HBc immunodominant loop, or a sequence of at least about 135 residues of the N-terminal 150 HBc amino acid residues,
(b) contains a C-terminal self-binding sequence of amino acid residues having a length of about 15 to about 35 residues, said self-binding sequence when present as an N-acetylated peptide forming self-assembled parallel multimers in aqueous PBS solution at pH 7.0 and a concentration of about 10 millimolar,
(c) contains a sequence of at least 5 amino acid residues from HBc position 135 to the HBc C-terminus,
said chimer molecules (i) containing no more than 10 percent conservatively substituted amino acid residues in the HBc sequence, and said particles being more stable than are particles formed from an otherwise identical HBc chimer that lacks said C-terminal self-binding sequence.

17. The recombinant HBc chimer protein molecule according to claim 16 wherein said peptide-bonded heterologous epitope or a heterologous linker residue for a conjugated epitope is a heterologous epitope.

18. The recombinant HBc chimer protein molecule according to claim 17 wherein said heterologous epitope is a B cell epitope.

19. The recombinant HBc chimer protein molecule according to claim 18 that contains a second heterologous epitope peptide-bonded to one of amino acid residues 1-4 of HBc.

20. The recombinant HBc chimer protein molecule according to claim 18 wherein said B cell epitope is peptide-bonded at a position in the HBc sequence between amino acid residues 76 and 85, and at least 5 residues of the HBc sequence of positions 76 through 85 are present.

21. The recombinant HBc chimer protein molecule according to claim 20 wherein the HBc sequence between amino acid residues 76 and 85 is present, but interrupted by said B cell epitope.

22. The recombinant HBc chimer protein molecule according to claim 17 further including a peptide-bonded heterologous T cell epitope.

23. A method for stabilizing supramolecular structures that comprises attaching a second self-binding peptide portion to a first polypeptide portion, wherein said first polypeptide portion self-assembles into organized, repetitive, supramolecular structures having at least about 9 subunits, and said second peptide portion comprises a peptide having a length of about 15 to about 80 amino acid residues, and said second peptide
a) when present as an N-acetylated peptide self-assembles to form parallel multimers in aqueous PBS solution at pH 7.0 and a concentration of about 10 millimoles/liter,
b) self assembles when present in solution at a concentration of at least ten micromoles/liter in the presence of a five-fold molar excess of a predetermined multivalent metal ion, or
c) is the self-assembling extracellular domain of the influenza A M2 polypeptide.

24. A method for stabilizing virus-like particles comprising:
a) preparing a chimeric polypeptide by covalently attaching a second self-binding peptide portion to a first polypeptide portion,
wherein said first polypeptide portion is a virus capsid polypeptide or is derived from a virus capsid polypeptide,
and said second peptide portion comprises a peptide having a length of about 15 to about 80 amino acid residues, said second peptide
i) when present as an N-acetylated peptide self-assembles to form parallel multimers in aqueous PBS solution at pH 7.0 and a concentration of about 10 millimoles/liter,
ii) self assembles when present in solution at a concentration of at least ten micromoles/liter in the presence of a five-fold molar excess of a predetermined multivalent metal ion, or
iii) is the self-assembling extracellular domain of the influenza A M2 polypeptide, with the proviso that the second portion is other than that extracellular M2 domain, which contains zero, one or two cysteines, N-terminally bonded to a first portion that is a HBc polypeptide; and
b) maintaining a plurality of said chimeric polypeptides in an aqueous composition for a time period sufficient for virus-like particles to assemble into virus-like particles having at least about 9 copies of said chimeric polypeptide; and
ii) recovering said virus like particles, said stabilized virus-like particles being more stable than are virus like particles comprising the virus capsid polypeptide lacking said attached self-binding peptide.

25. The supramolecular structures formed from the chimeric polypeptide according to claim 1.

26. The particles formed from the recombinant chimer hepatitis B core protein molecule according to claim 16.


SEQUENCE LISTING (338 sequences)

SEQ ID NO: 1 - protein, 15 aa, ARTIFICIAL SEQUENCE
  SELF-ASSEMBLING PEPTIDE WITH N-ACETYLATION AND COHN2 AT C TERMINUS
GLAQKLLEALQKALA

SEQ ID NO: 2 - protein, 16 aa, ARTIFICIAL SEQUENCE
  SELF-ASSEMBLING TRIHELIX PEPTIDE WITH N-ACYLATION AND C-TERMIAL CONH2
GELAEQKLEQALQKLA

SEQ ID NO: 3 - protein, 183 aa, Hepatitis B virus
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 4 - protein, 185 aa, Hepatitis B virus
MDIDPYKEFGATVELLSFLPSDFFPSVRDLLDTASALYREALESPEHCSPHHTALRQAILCWGELMTLATWVGNNLQDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 5 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNVGLKIRQLLWFHISCLTFGRETVLEYLVSFGVWIRTPPAYRPPNAPILSTLPETTVVRRRDRGRSPRRRTPSPRRRPSQSPRRRRSQSRESQC

SEQ ID NO: 6 - protein, 183 aa, Hepatitis B virus
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 7 - protein, 183 aa, woodchuck
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQC

SEQ ID NO: 8 - protein, 217 aa, ground squirrel
MYLFHLCLVFACVPCPTVQASKLCLGWLWDMDIDPYKEFGSSYQLLNFLPLDFFPDLNALVDTAAALYEEELTGREHCSPHHTAIRQALVCWEELTRLITWMSENTTEEVRRIIVDHVNNTWGLKVRQTLWFHLSCLTFGGHTVQEFLVSFGVWIRTPAPYRPPNAPILSTLPEHTVIRRRGGSRAARSPRRRTPSPRRRRSQSPRRRRSQSPASNC

SEQ ID NO: 9 - DNA, 78 bp, ARTIFICIAL SEQUENCE
  DNA CODING FOR INFLUENZA A M2 EXTERNAL DOMAIN
aatttctctgttaaccgaagtggagacgccgattcgtaacgaatggggtagccgctctaatgatagctctgacgagct

SEQ ID NO: 10 - protein, 33 aa, Artificial Sequence
  SELF-BINDING PEPTIDE GCN4-P1
RMKQLEDKVEELLSKNYHLENEVARLKKLVGER

SEQ ID NO: 11 - protein, 35 aa, ARTIFICIAL SEQUENCE
  SELF-BINDING PEPTIDE MAX
YQYMRKNHTHQQDIDDLKRQNALLEQQVRALEKAR

SEQ ID NO: 12 - protein, 31 aa, ARTIFICIAL SEQUENCE
  SELF-BINDING PEPTIDE
RKNDTLQQDIDDLKRQNALLEQQVRALEKAR

SEQ ID NO: 13 - protein, 32 aa, ARTIFICIAL SEQUENCE
  SELF-BINDING PEPTIDE
YLSVQAEEQKLISEEDLLRKRREQLKHKLEQL

SEQ ID NO: 14 - protein, 32 aa, ARTIFICIAL SEQUENCE
  SELF-BINDING PEPTIDE
YHSVQAEEHQLLLEKEKLQARQQQLLKKIEHA

SEQ ID NO: 15 - protein, 32 aa, ARTIFICIAL SEQUENCE
  SELF-BINDING PEPTIDE
LQALVGAEKRMATEKRQLRCRQQQLQKRIAYL

SEQ ID NO: 16 - protein, 25 aa, ARTIFICIAL SEQUENCE
  SELF-BINDING PEPTIDE
IFSLEQEKTRLLQQNTQLKRFIQEL

SEQ ID NO: 17 - protein, 42 aa, ARTIFICIAL SEQUENCE
  SELF-BINDING PEPTIDE
IQELRQSNHQLQLDNDVLRQQVEDLKNKNLLLRLSEELQGLD

SEQ ID NO: 18 - protein, 33 aa, ARTIFICIAL SEQUENCE
  SELF-BINDING PEPTIDE
RIKQLEDKIEELLSKIYHLENEIARLKKLVGER

SEQ ID NO: 19 - protein, 33 aa, ARTIFICIAL SEQUENCE
  SELF-BINDING PEPTIDE
RIKQIEDKIEEILSKIYHIENEIARIKKLVGER

SEQ ID NO: 20 - protein, 33 aa, ARTIFICIAL SEQUENCE
  SELF-BINDING PEPTIDE
RLKQIEDKLEEILSKLYHIENELARIKKLVGER

SEQ ID NO: 21 - protein, 33 aa, ARTIFICIAL SEQUENCE
  SELF-BINDING PEPTIDE
RVKQIEDKVEEILSKVYHIENEVARIKKLVGER

SEQ ID NO: 22 - protein, 33 aa, ARTIFICIAL SEQUENCE
  SELF-BINDING PEPTIDE
RLKQVEDKLEEVLSKLYHVENELARVKKLVGER

SEQ ID NO: 23 - protein, 33 aa, ARTIFICIAL SEQUENCE
  SELF-BINDING PEPTIDE
RVKQLEDKVEELLSKVYHLENEVARLKKLVGER

SEQ ID NO: 24 - protein, 33 aa, ARTIFICIAL SEQUENCE
  SELF-BINDING PEPTIDE GCN4-LL
RLKQLEDKLEELLSKLYHLENELARLKKLVGER

SEQ ID NO: 25 - protein, 15 aa, ARTIFICIAL SEQUENCE
  SELF-BINDING PEPTIDE
GLAQKLLEALQKALA

SEQ ID NO: 26 - protein, 16 aa, ARTIFICIAL SEQUENCE
  SELF-BINDING PEPTIDE
GELAEQKLEQALQKLA

SEQ ID NO: 27 - protein, 52 aa, ARTIFICIAL SEQUENCE
  SELF-BINDING PEPTIDE RHCC
GSIINETADDIVYRLTVIIDDRYESLKNLITLRADRLEMIINDNVSTILASI

SEQ ID NO: 28 - protein, 45 aa, ARTIFICIAL SEQUENCE
  SELF-BINDING PEPTIDE
MDLAPQMLRELQETNAALQDVRELLRQQVKEITFLKNTVMECDAC

SEQ ID NO: 29 - protein, 44 aa, ARTIFICIAL SEQUENCE
  SELF-BINDING PEPTIDE
DLGPQMLRELQETNAALQDVRDWLRQQVREITFLKNTVMECDAC

SEQ ID NO: 30 - protein, 26 aa, ARTIFICIAL SEQUENCE
  SELF-BINDING PEPTIDE
ISLLTEVETPIRNEWGSRSNDSSDEL

SEQ ID NO: 31 - protein, 15 aa, Streptococcus pneumoniae
KLEELSDKIDELDAE

SEQ ID NO: 32 - protein, 35 aa, Streptococcus pneumoniae
QKKYDEDQKKTEEKAALEKAASEEMDKAVAAVQQA

SEQ ID NO: 33 - protein, 27 aa, Cryptosporidium parvum
QDKPADAPAAEAPAAEPAAQQDKPADA

SEQ ID NO: 34 - protein, 17 aa, Human immunodeficiency virus
RKRIHIGPGRAFYITKN

SEQ ID NO: 35 - protein, 42 aa, HUMAN IMMUNODEFICIENCY VIRUS
SNCTRPNNNTRKSIRIQRGPGRAFVTIGKIGNMRQAHCNISG

SEQ ID NO: 36 - protein, 42 aa, HUMAN IMMUNODEFICIENCY VIRUS
SNCTRPNNNTRKRIRIQRGPGRAFVTIGKIGNMRQAHCNISG

SEQ ID NO: 37 - protein, 41 aa, HUMAN IMMUNODEFICIENCY VIRUS
SNCTRPNYNKRKRIHIGPGRAFYTTKNIIGTIRQAHCNISG

SEQ ID NO: 38 - protein, 39 aa, HUMAN IMMUNODEFICIENCY VIRUS
SNCTRPGNNTRRGIHFGPGQALYTTGIVDIRRAYCTING

SEQ ID NO: 39 - protein, 41 aa, HUMAN IMMUNODEFICIENCY VIRUS
SNCTRPNNNTRKSITKGPGRVIYATGQIIGDIRAHCNLSGS

SEQ ID NO: 40 - protein, 40 aa, HUMAN IMMUNODEFICIENCY VIRUS
STCARPYQNTRQRTPIGLGQSLYTTRSRSIIGQAHCNISG

SEQ ID NO: 41 - protein, 40 aa, HUMAN IMMUNODEFICIENCY VIRUS
SNCTRPGNNTRRGIHFGPGQALYTTGIVDEIRRAYCNISG

SEQ ID NO: 42 - protein, 42 aa, HUMAN IMMUNODEFICIENCY VIRUS
SNCTRPNNTRKSITKQRGPGRVLYATGQIIGDIRKAHCNSIG

SEQ ID NO: 43 - protein, 41 aa, HUMNA IMMUNODEFICIENCY VIRUS
STCARPYQNTRQRTPIGLGQSLYTTRGRTKIIGQAHCNISG

SEQ ID NO: 44 - protein, 31 aa, Foot-and-mouth disease virus
YNGECRYNRNAVPNLRGDLQVLAQKVARTLP

SEQ ID NO: 45 - protein, 10 aa, Influenza A virus
YRNLLWLTEK

SEQ ID NO: 46 - protein, 23 aa, Influenza A virus
SLLTEVETPIRNEWGCRCNGSSD

SEQ ID NO: 47 - protein, 23 aa, Influenza A virus
SLLTEVETPIRNEWGCRCNDSSD

SEQ ID NO: 48 - protein, 23 aa, Influenza A virus
SLLTEVETPIRNEWGARANDSSD

SEQ ID NO: 49 - protein, 19 aa, Influenza A virus
EQQSAVDADDSHFVSIELE

SEQ ID NO: 50 - protein, 23 aa, Influenza A virus
SLLTEVETPIRNEWGSRSNDSSD

SEQ ID NO: 51 - protein, 23 aa, Influenza A virus
SLLTEVETPIRNEWGSRCNDSSD

SEQ ID NO: 52 - protein, 23 aa, Influenza A virus
SLLTEVETPIRNEWGCRSNDSSD

SEQ ID NO: 53 - protein, 23 aa, Influenza A virus
SLLTEVETPIRNEWGCRANDSSD

SEQ ID NO: 54 - protein, 23 aa, Influenza A virus
SLLTEVETPIRNEWGARCNDSSD

SEQ ID NO: 55 - protein, 24 aa, Influenza A virus
MSLLTEVETPIRNEWGCRCNDSSD

SEQ ID NO: 56 - protein, 24 aa, Influenza A virus
MSLLTEVETPIRNEWGSRSNDSSD

SEQ ID NO: 57 - protein, 35 aa, Influenza A virus
MGISLLTEVETPIRNEWGCRCNDSSDELLGWLWGI

SEQ ID NO: 58 - protein, 24 aa, Influenza A virus
MSLLTEVETPIRNEWGARANDSSD

SEQ ID NO: 59 - protein, 24 aa, Influenza A virus
MSLLTEVETPIRNEWGCRANDSSD

SEQ ID NO: 60 - protein, 24 aa, Influenza A virus
MSLLTEVETPIRNEWGARCNDSSD

SEQ ID NO: 61 - protein, 24 aa, Influenza A virus
MSLLTEVETPIRNEWGCRSNDSSD

SEQ ID NO: 62 - protein, 24 aa, Influenza A virus
MSLLTEVETPIRNEWGSRCNDSSD

SEQ ID NO: 63 - protein, 46 aa, Influenza A virus
SLLTEVETPIRNEWGSRSNDSSDSLLTEVETPIRNEWGSRSNDSSD

SEQ ID NO: 64 - protein, 69 aa, Influenza A virus
SLLTEVETPIRNEWGSRSNDSSDSLLTEVETPIRNEWGSRSNDSSDSLLTEVETPIRNEWGSRSNDSSD

SEQ ID NO: 65 - protein, 46 aa, Influenza A virus
SLLTEVETPIRNEWGARANDSSDSLLTEVETPIRNEWGARANDSSD

SEQ ID NO: 66 - protein, 69 aa, Influenza A virus
SLLTEVETPIRNEWGARANDSSDSLLTEVETPIRNEWGARANDSSDSLLTEVETPIRNEWGARANDSSD

SEQ ID NO: 67 - protein, 19 aa, Influenza A virus
EVETPIRNEWGSRCNDSSD

SEQ ID NO: 68 - protein, 38 aa, Influenza A virus
EVETPIRNEWGSRCNDSSDEVETPIRNEWGSRCNDSSD

SEQ ID NO: 69 - protein, 57 aa, Influenza  A virus
EVETPIRNEWGSRCNDSSDEVETPIRNEWGSRCNDSSDEVETPIRNEWGSRCNDSSD

SEQ ID NO: 70 - protein, 23 aa, Influenza A virus
  MISC_FEATURE (1)..(1): Xaa at position 1 is methionine or absent. If methionine then Xaa in positions 2 through 8 are not absent
  MISC_FEATURE (2)..(2): Xaa at position 2 is serine or absent.  If serine then Xaa in positions 3 through 8 are not absent.
  MISC_FEATURE (3)..(3): Xaa at position 3 is leucine or absent.  If leucine then Xaa in positions 4 through 8 are not absent.
  MISC_FEATURE (4)..(4): Xaa at position 4 is leucine or absent.  If leucine then Xaa in positions 5 through 8 are not absent.
  MISC_FEATURE (5)..(5): Xaa at position 5 is threonine or absent.  If threonine than Xaa in positions 6 through 8 are not absent.
  MISC_FEATURE (6)..(6): Xaa at position 6 is glutamic acid or absent. If glutamic acid then Xaa in positions 7 through 8 are not absent.
  MISC_FEATURE (7)..(7): Xaa at position 7 is valine or absent.  If valine then Xaa in position 8 is not absent.
  misc_feature (8)..(8): Xaa can be any naturally occurring amino acid
  MISC_FEATURE (15)..(15): Xaa at position 15 is tryptophan or absent.
  MISC_FEATURE (16)..(16): Xaa at position 16 is glycine or absent.  If glycine then Xaa in position 15 is not absent.
  MISC_FEATURE (17)..(17): Xaa at position 17 is absent or present, if present Xaa in position 17 is cysteine, serine or alanine.  If Xaa in position 17 is present then positions 15 through 16 are not absent.
  MISC_FEATURE (18)..(18): Xaa at position 18 is arginine or absent.  If arginine then Xaa in positions 15 through 17 are not absent.
  MISC_FEATURE (19)..(19): Xaa at position 19 is absent or present, if present Xaa in position 19 is cysteine, serine or alanine.  If Xaa in position 19 is present then positions 15 through 18 are not absent.
  MISC_FEATURE (20)..(20): Xaa at position 20 is asparagine or absent. If asparagine then Xaa in positions 15 through 19 are not absent.
  MISC_FEATURE (21)..(21): Xaa at position 21 is aspartic acid or absent. If aspartic acid then Xaa in positions 15 through 20 are not absent.
  MISC_FEATURE (22)..(22): Xaa at position 22 is serine or absent.  If serine then Xaa in positions 15 through 21 are not absent.
  MISC_FEATURE (23)..(23): Xaa at position 23 is serine or absent.  If serine then Xaa in positions 15 through 22 are not absent.
XXXXXXXXTPIRNEXXXXXXXXX

SEQ ID NO: 71 - protein, 47 aa, Influenza A virus
  MISC_FEATURE (1)..(1): Xaa at position 1 is methionine or absent.  If methionine then Xaa in positions 2 through 8 are not absent
  MISC_FEATURE (2)..(2): Xaa at position 2 is serine or absent.  If serine then Xaa in positions 3 through 8 are not absent.
  MISC_FEATURE (3)..(3): Xaa at position 3 is leucine or absent.  If leucine then Xaa in positions 4 through 8 are not absent.
  MISC_FEATURE (4)..(4): Xaa at position 4 is leucine or absent.  If leucine then Xaa in positions 5 through 8 are not absent.
  MISC_FEATURE (5)..(5): Xaa at position 5 is threonine or absent.  If threonine than Xaa in positions 6 through 8 are not absent.
  MISC_FEATURE (6)..(6): Xaa at position 6 is glutamic acid or absent. If glutamic acid then Xaa in positions 7 through 8 are not absent.
  MISC_FEATURE (7)..(7): Xaa at position 7 is valine or absent.  If valine then Xaa in position 8 is not absent.
  MISC_FEATURE (8)..(8): Xaa at position 8 is glutamic acid or absent.
  MISC_FEATURE (15)..(15): Xaa at position 15 is tryptophan or absent.
  MISC_FEATURE (16)..(16): Xaa at position 16 is glycine or absent.  If glycine then Xaa in position 15 is not absent.
  MISC_FEATURE (17)..(17): Xaa at position 17 is absent or present, if present Xaa in position 17 is cysteine, serine or alanine.  If Xaa in position 17 is present then positions 15 through 16 are not absent.
  MISC_FEATURE (18)..(18): Xaa at position 18 is arginine or absent.  If arginine then Xaa in positions 15 through 17 are not absent.
  MISC_FEATURE (19)..(19): Xaa at position 19 is absent or present, if present Xaa in position 19 is cysteine, serine or alanine.  If Xaa in position 19 is present then positions 15 through 18 are not absent.
  MISC_FEATURE (20)..(20): Xaa at position 20 is asparagine or absent. If asparagine then Xaa in positions 15 through 19 are not absent.
  MISC_FEATURE (21)..(21): Xaa at position 21 is aspartic acid or absent. If aspartic acid then Xaa in positions 15 through 20 are not absent.
  MISC_FEATURE (22)..(22): Xaa at position 22 is serine or absent.  If serine then Xaa in positions 15 through 21 are not absent.
  MISC_FEATURE (23)..(23): Xaa at position 23 is serine or absent.  If serine then Xaa in positions 15 through 22 are not absent.
  MISC_FEATURE (24)..(24): Xaa at position 24 is aspartic acid or absent. If aspartic acid then Xaa in positions 15 through 23 are not absent.
  MISC_FEATURE (25)..(25): Xaa at position 25 is serine or absent.  If serine then Xaa in positions 26 through 31 are not absent
  MISC_FEATURE (26)..(26): Xaa at position 26 is leucine or absent.  If serine then Xaa in positions 27 through 31 are not absent
  MISC_FEATURE (27)..(27): Xaa at position 27 is leucine or absent.  If leucine then Xaa in positions 28 through 31 are not absent.
  MISC_FEATURE (28)..(28): Xaa at position 28 is threonine or absent.  If threonine than Xaa in positions 29 through 31 are not absent.
  MISC_FEATURE (29)..(29): Xaa at position 29 is glutamic acid or absent. If glutamic acid then Xaa in positions 30 through 31 are not absent.
  MISC_FEATURE (30)..(30): Xaa at position 30 is valine or absent.  If valine then Xaa in position 31 is not absent.
  MISC_FEATURE (31)..(31): Xaa at position 31 is glutamic acid or absent.
  MISC_FEATURE (38)..(38): Xaa at position 38 is tryptophan or absent.
  MISC_FEATURE (39)..(39): Xaa at position 39 is glycine or absent.  If glycine then Xaa in position 38 is not absent.
  MISC_FEATURE (40)..(40): Xaa at position 40 is absent or present, if present Xaa in position 40 is cysteine, serine or alanine.  If Xaa in position 40 is present then positions 38 through 39 are not absent.
  MISC_FEATURE (41)..(41): Xaa at position 41 is arginine or absent.  If arginine then Xaa in positions 38 through 40 are not absent
  MISC_FEATURE (42)..(42): Xaa at position 42 is absent or present, if present Xaa in position 42 is cysteine, serine or alanine.  If Xaa in position 42 is present then positions 38 through 41 are not absent.
  MISC_FEATURE (43)..(43): Xaa at position 43 is asparagine or absent. If asparagine then Xaa in positions 38 through 42 are not absent.
  MISC_FEATURE (44)..(44): Xaa at position 44 is aspartic acid or absent. If aspartic acid then Xaa in positions 38 through 43 are not absent.
  misc_feature (45)..(45): Xaa can be any naturally occurring amino acid
  MISC_FEATURE (46)..(46): Xaa at position 46 is serine or absent.  If serine then Xaa in positions 38 through 45 are not absent.
  MISC_FEATURE (47)..(47): Xaa at position 47 is aspartic acid or absent. If aspartic acid then Xaa in positions 38 through 46 are not absent.
XXXXXXXXTPIRNEXXXXXXXXXXXXXXXXXTPIRNEXXXXXXXXXX

SEQ ID NO: 72 - protein, 70 aa, Influenza A virus
  MISC_FEATURE (1)..(1): Xaa at position 1 is methionine or absent.  If methionine then Xaa in positions 2 through 8 are not absent
  MISC_FEATURE (2)..(2): Xaa at position 2 is serine or absent.  If serine then Xaa in positions 3 through 8 are not absent.
  MISC_FEATURE (3)..(3): Xaa at position 3 is leucine or absent.  If leucine then Xaa in positions 4 through 8 are not absent.
  MISC_FEATURE (4)..(4): Xaa at position 4 is leucine or absent.  If leucine then Xaa in positions 5 through 8 are not absent.
  MISC_FEATURE (5)..(5): Xaa at position 5 is threonine or absent.  If threonine than Xaa in positions 6 through 8 are not absent.
  MISC_FEATURE (6)..(6): Xaa at position 6 is glutamic acid or absent. If glutamic acid then Xaa in positions 7 through 8 are not absent.
  MISC_FEATURE (7)..(7): Xaa at position 7 is valine or absent.  If valine then Xaa in position 8 is not absent.
  MISC_FEATURE (8)..(8): Xaa at position 8 is glutamic acid or absent.
  MISC_FEATURE (15)..(15): Xaa at position 15 is tryptophan or absent.
  MISC_FEATURE (16)..(16): Xaa at position 16 is glycine or absent.  If glycine then Xaa in position 15 is not absent.
  MISC_FEATURE (17)..(17): Xaa at position 17 is absent or present, if present Xaa in position 17 is cysteine, serine or alanine.  If Xaa in position 17 is present then positions 15 through 16 are not absent.
  MISC_FEATURE (18)..(18): Xaa at position 18 is arginine or absent.  If arginine then Xaa in positions 15 through 17 are not absent.
  MISC_FEATURE (19)..(19): Xaa at position 19 is absent or present, if present Xaa in position 19 is cysteine, serine or alanine.  If Xaa in position 19 is present then positions 15 through 18 are not absent.
  MISC_FEATURE (20)..(20): Xaa at position 20 is asparagine or absent. If asparagine then Xaa in positions 15 through 19 are not absent.
  MISC_FEATURE (21)..(21): Xaa at position 21 is aspartic acid or absent. If aspartic acid then Xaa in positions 15 through 20 are not absent.
  MISC_FEATURE (22)..(22): Xaa at position 22 is serine or absent.  If serine then Xaa in positions 15 through 21 are not absent.
  MISC_FEATURE (23)..(23): Xaa at position 23 is serine or absent.  If serine then Xaa in positions 15 through 22 are not absent.
  MISC_FEATURE (24)..(24): Xaa at position 24 is aspartic acid or absent. If aspartic acid then Xaa in positions 15 through 23 are not absent.
  MISC_FEATURE (25)..(25): Xaa at position 25 is serine or absent.  If serine then Xaa in positions 26 through 31 are not absent
  MISC_FEATURE (26)..(26): Xaa at position 26 is leucine or absent.  If leucine then Xaa in positions 27 through 31 are not absent.
  MISC_FEATURE (27)..(27): Xaa at position 27 is leucine or absent.  If leucine then Xaa in positions 28 through 31 are not absent.
  MISC_FEATURE (28)..(28): Xaa at position 28 is threonine or absent. If threonine than Xaa in positions 29 through 31 are not absent.
  MISC_FEATURE (29)..(29): Xaa at position 29 is glutamic acid or absent. If glutamic acid then Xaa in positions 30 through 31 are not absent.
  MISC_FEATURE (30)..(30): Xaa at position 30 is valine or absent.  If valine then Xaa in position 31 is not absent.
  MISC_FEATURE (31)..(31): Xaa at position 31 is glutamic acid or absent.
  MISC_FEATURE (38)..(38): Xaa at position 38 is tryptophan or absent.
  MISC_FEATURE (39)..(39): Xaa at position 39 is glycine or absent.  If glycine then Xaa in position 38 is not absent.
  MISC_FEATURE (40)..(40): Xaa at position 40 is absent or present, if present Xaa in position 40 is cysteine, serine or alanine.  If Xaa in position 40 is present then positions 38 through 39 are not absent.
  MISC_FEATURE (41)..(41): Xaa at position 41 is arginine or absent.  If arginine then Xaa in positions 38 through 40 are not absent.
  MISC_FEATURE (42)..(42): Xaa at position 42 is absent or present, if present Xaa in position 42 is cysteine, serine or alanine.  If Xaa in position 42 is present then positions 38 through 41 are not absent.
  MISC_FEATURE (43)..(43): Xaa at position 43 is asparagine or absent. If asparagine then Xaa in positions 38 through 42 are not absent.
  MISC_FEATURE (44)..(44): Xaa at position 44 is aspartic acid or absent. If aspartic acid then Xaa in positions 38 through 43 are not absent.
  MISC_FEATURE (45)..(45): Xaa at position 45 is serine or absent.  If serine then Xaa in positions 38 through 44 are not absent.
  MISC_FEATURE (46)..(46): Xaa at position 46 is serine or absent.  If serine then Xaa in positions 38 through 45 are not absent.
  MISC_FEATURE (47)..(47): Xaa at position 47 is aspartic acid or absent. If aspartic acid then Xaa in positions 38 through 46 are not absent.
  MISC_FEATURE (48)..(48): Xaa at position 48 is serine or absent.  If serine then Xaa in positions 49 through 54 are not absent.
  MISC_FEATURE (49)..(49): Xaa at position 49 is leucine or absent.  If leucine then Xaa in positions 50 through 54 are not absent.
  MISC_FEATURE (50)..(50): Xaa at position 50 is leucine or absent.  If leucine then Xaa in positions 51 through 54 are not absent.
  MISC_FEATURE (51)..(51): Xaa at position 51 is threonine or absent.  If threonine than Xaa in positions 52 through 54 are not absent.
  MISC_FEATURE (52)..(52): Xaa at position 52 is glutamic acid or absent. If glutamic acid then Xaa in positions 53 through 54 are not absent.
  MISC_FEATURE (53)..(53): Xaa at position 53 is valine or absent.  If valine then Xaa in position 54 is not absent.
  MISC_FEATURE (54)..(54): Xaa at position 54 is glutamic acid or absent
  MISC_FEATURE (61)..(61): Xaa at position 61 is tryptophan or absent.
  MISC_FEATURE (62)..(62): Xaa at position 62 is glycine or absent.  If glycine then Xaa in position 61 is not absent.
  MISC_FEATURE (63)..(63): Xaa at position 63 is absent or present, if present Xaa in position 63 is cysteine, serine or alanine.  If Xaa in position 63 is present then positions 61 through 62 are not absent.
  MISC_FEATURE (64)..(64): Xaa at position 64 is arginine or absent.  If arginine then Xaa in positions 61 through 63 are not absent
  MISC_FEATURE (65)..(65): Xaa at position 65 is absent or present, if present Xaa in position 65 is cysteine, serine or alanine.  If Xaa in position 65 is present then positions 61 through 64 are not absent.
  MISC_FEATURE (66)..(66): Xaa at position 66 is asparagine or absent. If asparagine then Xaa in positions 61 through 65 are not absent.
  MISC_FEATURE (67)..(67): Xaa at position 67 is aspartic acid or absent. If aspartic acid then Xaa in positions 61 through 66 are not absent.
  MISC_FEATURE (68)..(68): Xaa at position 68 is serine or absent.  If serine then Xaa in positions 61 through 67 are not absent.
  MISC_FEATURE (69)..(69): Xaa at position 69 is serine or absent.  If serine then Xaa in positions 61 through 68 are not absent.
  MISC_FEATURE (70)..(70): Xaa at position 70 is aspartic acid or absent. If aspartic acid then Xaa in positions 61 through 69 are not absent.
XXXXXXXXTPIRNEXXXXXXXXXXXXXXXXXTPIRNEXXXXXXXXXXXXXXXXXTPIRNEXXXXXXXXXX

SEQ ID NO: 73 - protein, 24 aa, Influenza A virus
  MISC_FEATURE (1)..(1): Xaa at position 1 is methionine or absent.  If methionine then Xaa in position 2 through 8 are not absent.
  MISC_FEATURE (2)..(2): Xaa at position 2 is serine or absent.  If serine then Xaa in position 3 through 8 are not absent.
  MISC_FEATURE (3)..(3): Xaa at position 3 is leucine or absent.  If leucine then Xaa in position 4 through 8 are not absent.
  MISC_FEATURE (4)..(4): Xaa at position 4 is leucine or absent.  If leucine then Xaa in position 5 through 8 are not absent.
  MISC_FEATURE (5)..(5): Xaa at position 4 is threonine or proline or absent.  If threonine or proline then Xaa in position 6 through 8 are not absent.
  MISC_FEATURE (6)..(6): Xaa at position 6 is glutamic acid or absent. If glutamic acid then Xaa in position 7 through 8 are not absent.
  MISC_FEATURE (7)..(7): Xaa at position 7 is valine or absent.  If valine then Xaa in position 8 is not absent.
  MISC_FEATURE (8)..(8): Xaa at position 8 is glutamic acid or aspartic acid or absent.
  MISC_FEATURE (10)..(10): Xaa at position 10 is proline, leucine or histidine.
  MISC_FEATURE (11)..(11): Xaa at position 11 is isoleucine or threonine.
  MISC_FEATURE (13)..(13): Xaa at position 13 is asparagine or serine.
  MISC_FEATURE (14)..(14): Xaa at position 14 is glutamic acid or glycine.
  MISC_FEATURE (15)..(15): Xaa at position 15 is tryptophan or absent.
  MISC_FEATURE (16)..(16): Xaa at position 16 is glycine, glutamic acid or absent.  If glycine or glutamic acid then Xaa in position 16 is not absent.
  MISC_FEATURE (17)..(17): Xaa at position 17 is absent or present, if present Xaa in position 17 is cysteine, serine or alanine.  If Xaa in position 17 is present then positions 15 through 16 are not absent.
  MISC_FEATURE (18)..(18): Xaa at position 18 arginine, lysine or absent. If arginine or lysine then Xaa in positions 15 through 17 are not absent.
  MISC_FEATURE (19)..(19): Xaa at position 19 is absent or present, if present Xaa in position 19 is cysteine, serine or alanine.  If Xaa is position 19 is present then positions 15 through 18 are not absent.
  MISC_FEATURE (20)..(20): Xaa at position 20 is asparagine, serine, glycine or absent.  If asparagine or serine or glycine then Xaa is positions 15 through 19 are not absent.
  MISC_FEATURE (21)..(21): Xaa at position 21 is aspartic acid, glycine or absent.  If aspartic acid or glycine then Xaa is positions 15 through 20 are not absent.
  MISC_FEATURE (22)..(22): Xaa at position 22 is serine or absent.  If serine then Xaa is positions 15 through 21 are not absent.
  MISC_FEATURE (23)..(23): Xaa at position 23 is serine or absent.  If serine then Xaa is positions 15 through 22 are not absent.
  MISC_FEATURE (24)..(24): Xaa at position 24 is aspartic acid or absent. If aspartic acid then Xaa is positions 15 through 23 are not absent.
XXXXXXXXTXXRXXXXXXXXXXXX

SEQ ID NO: 74 - protein, 47 aa, Influenza A virus
  MISC_FEATURE (1)..(1): Xaa at position 1 is methionine or absent. If methionine then Xaa in position 2 through 8 are not absent.
  MISC_FEATURE (2)..(2): Xaa at position 2 is serine or absent.  If serine then Xaa in position 3 through 8 are not absent.
  MISC_FEATURE (3)..(3): Xaa at position 3 is leucine or absent.  If leucine then Xaa in position 4 through 8 are not absent.
  MISC_FEATURE (4)..(4): Xaa at position 4 is leucine or absent.  If leucine then Xaa in position 5 through 8 are not absent.
  MISC_FEATURE (5)..(5): Xaa at position 4 is threonine or absent.  If threonine then Xaa in position 6 through 8 are not absent.
  MISC_FEATURE (6)..(6): Xaa at position 6 is glutamic acid or absent. If glutamic acid then Xaa in position 7 through 8 are not absent.
  MISC_FEATURE (7)..(7): Xaa at position 7 is valine or absent.  If valine then Xaa in position 8 is not absent.
  MISC_FEATURE (8)..(8): Xaa at position 8 is glutamic acid or aspartic acid or absent.
  MISC_FEATURE (10)..(10): Xaa at position 10 is proline, leucine or histidine.
  MISC_FEATURE (11)..(11): Xaa at position 11 is isoleucine or threonine.
  MISC_FEATURE (13)..(13): Xaa at position 13 is asparagine or serine.
  MISC_FEATURE (14)..(14): Xaa at position 14 is glutamic acid or glycine.
  MISC_FEATURE (15)..(15): Xaa at position 15 is tryptophan or absent.
  MISC_FEATURE (16)..(16): Xaa at position 16 is glycine, glutamic acid or absent.  If glycine or glutamic acid then Xaa in position 16 is not absent.
  MISC_FEATURE (17)..(17): Xaa at position 17 is absent or present, if present Xaa in position 17 is cysteine, serine or alanine.  If Xaa in position 17 is present then positions 15 through 16 are not absent.
  MISC_FEATURE (18)..(18): Xaa at position 18 arginine, lysine or absent. If arginine or lysine then Xaa in positions 15 through 17 are not absent.
  MISC_FEATURE (19)..(19): Xaa at position 19 is absent or present, if present Xaa in position 19 is cysteine, serine or alanine.  If Xaa is position 19 is present then positions 15 through 18 are not absent.
  MISC_FEATURE (20)..(20): Xaa at position 20 is asparagine, serine or glycine or absent. If asparagine or serine then Xaa is positions 15 through 19 are not absent.
  MISC_FEATURE (21)..(21): Xaa at position 21 is aspartic acid, glycine or absent.  If aspartic acid or glycine then Xaa is positions 15 through 20 are not absent.
  MISC_FEATURE (22)..(22): Xaa at position 22 is serine or absent.  If serine then Xaa is positions 15 through 21 are not absent.
  MISC_FEATURE (23)..(23): Xaa at position 23 is serine or absent.  If serine then Xaa is positions 15 through 22 are not absent.
  MISC_FEATURE (24)..(24): Xaa at position 24 is aspartic acid or absent. If aspartic acid then Xaa is positions 15 through 23 are not absent.
  MISC_FEATURE (25)..(25): Xaa at position 25 is serine or absent.  If serine then Xaa in positions 26 through 31 are not absent.
  MISC_FEATURE (26)..(26): Xaa at position 26 is leucine or absent.  If leucine then Xaa in positions 27 through 31 are not absent.
  MISC_FEATURE (27)..(27): Xaa at position 27 is leucine or absent.  If leucine then Xaa in positions 28 through 31 are not absent.
  MISC_FEATURE (28)..(28): Xaa at position 28 is threonine, proline or absent.  If threonine or proline then Xaa in positions 29 through 31 are not absent.
  MISC_FEATURE (29)..(29): Xaa at position 29 is glutamic acid or absent. If glutamic acid then Xaa in positions 30 through 31 are not absent.
  MISC_FEATURE (30)..(30): Xaa at position 30 is valine or absent.  If valine then Xaa in position 31 is not absent.
  MISC_FEATURE (31)..(31): Xaa at position 31 is glutamic acid or aspartic acid or absent.
  MISC_FEATURE (33)..(33): Xaa at position 33 is proline, leucine or histidine.
  MISC_FEATURE (34)..(34): Xaa at position 34 is isoleucine or threonine
  MISC_FEATURE (36)..(36): Xaa at position 36 is asparagine or serine.
  MISC_FEATURE (37)..(37): Xaa at position 37 is glumatic acid or glycine.
  MISC_FEATURE (38)..(38): Xaa at position 38 is tryptophan or absent.
  MISC_FEATURE (39)..(39): Xaa at position 39 is glycine, glutamic acid or absent.  If glycine or glutamic acid then Xaa in position 38 is not absent.
  MISC_FEATURE (40)..(40): Xaa at position 40 is absent of present, if present Xaa in position 40 is cysteine, serine or alanine.  If Xaa in position 40 is present then positions 38 through 39 are not absent.
  MISC_FEATURE (41)..(41): Xaa at position 41 is arginine, lysine or absent.  If arginine or lysine than Xaa in positions 38 through 40 are not absent.
  MISC_FEATURE (42)..(42): Xaa at position 42 is absent or present, if present Xaa in position 42 is cysteine, serine or alanine.  If Xaa in position 42 is present then positions 38 through 41 are not absent.
  MISC_FEATURE (43)..(43): Xaa at position 43 is asparagine, serine or absent.  If asparagine or serine then Xaa in positions 38 through 42 are not absent.
  MISC_FEATURE (44)..(44): Xaa at position 44 is aspartic acid, glycine or absent.  If aspartic acid or glycine then Xaa in positions 38 through 43 are not absent.
  MISC_FEATURE (45)..(45): Xaa at position 45 is serine or absent.  If serine then Xaa in positions 38 through 44 are not absent.
  MISC_FEATURE (46)..(46): Xaa at position 46 is serine or absent.  If serine then Xaa in positions 38 through 45 are not absent.
  MISC_FEATURE (47)..(47): Xaa at position 47 is aspartic acid or absent. If aspartic acid then Xaa in positions 38 through 46 are not absent.
XXXXXXXXTXXRXXXXXXXXXXXXXXXXXXXTXXRXXXXXXXXXXXX

SEQ ID NO: 75 - protein, 70 aa, Influenza A virus
  MISC_FEATURE (1)..(1): Xaa at position 1 is methionine or absent. If methionine then Xaa in position 2 through 8 are not absent.
  MISC_FEATURE (2)..(2): Xaa at position 2 is serine or absent.  If serine then Xaa in position 3 through 8 are not absent.
  MISC_FEATURE (3)..(3): Xaa at position 3 is leucine or absent.  If leucine then Xaa in position 4 through 8 are not absent.
  MISC_FEATURE (4)..(4): Xaa at position 4 is leucine or absent.  If leucine then Xaa in position 5 through 8 are not absent.
  MISC_FEATURE (5)..(5): Xaa at position 5 is threonine or absent.  If threonine then Xaa in position 6 through 8 are not absent.
  MISC_FEATURE (6)..(6): Xaa at position 6 is glutamic acid or absent. If glutamic acid then Xaa in position 7 through 8 are not absent.
  MISC_FEATURE (7)..(7): Xaa at position 7 is valine or absent.  If valine then Xaa in position 8 is not absent.
  MISC_FEATURE (8)..(8): Xaa at position 8 is glutamic acid or absent.
  MISC_FEATURE (10)..(10): Xaa at position 10 is proline, leucine or histidine.
  MISC_FEATURE (11)..(11): Xaa at position 11 is isoleucine or threonine.
  MISC_FEATURE (13)..(13): Xaa at position 13 is asparagine or serine.
  MISC_FEATURE (14)..(14): Xaa at position 14 is glutamic acid or glycine.
  MISC_FEATURE (15)..(15): Xaa at position 15 is tryptophan or absent.
  MISC_FEATURE (16)..(16): Xaa at position 16 is glycine, glutamic acid or absent.  If glycine or glutamic acid then Xaa in position 16 is not absent.
  MISC_FEATURE (17)..(17): Xaa at position 17 is absent or present, if present Xaa in position 17 is cysteine, serine or alanine.  If Xaa in position 17 is present then positions 15 through 16 are not absent.
  MISC_FEATURE (18)..(18): Xaa at position 18 arginine, lysine or absent. If arginine or lysine then Xaa in positions 15 through 17 are not absent.
  MISC_FEATURE (19)..(19): Xaa at position 19 is absent or present, if present Xaa in position 19 is cysteine, serine or alanine.  If Xaa is position 19 is present then positions 15 through 18 are not absent.
  MISC_FEATURE (20)..(20): Xaa at position 20 is asparagine, serine, glycine or absent.  If asparagine or serine then Xaa is positions 15 through 19 are not absent.
  MISC_FEATURE (21)..(21): Xaa at position 21 is aspartic acid, glycine or absent.  If aspartic acid or glycine then Xaa is positions 15 through 20 are not absent.
  MISC_FEATURE (22)..(22): Xaa at position 22 is serine or absent.  If serine then Xaa is positions 15 through 21 are not absent.
  MISC_FEATURE (23)..(23): Xaa at position 23 is serine or absent.  If serine then Xaa is positions 15 through 22 are not absent.
  MISC_FEATURE (24)..(24): Xaa at position 24 is aspartic acid or absent. If aspartic acid then Xaa is positions 15 through 23 are not absent.
  MISC_FEATURE (25)..(25): Xaa at position 25 is serine or absent.  If serine then Xaa in positions 26 through 31 are not absent.
  MISC_FEATURE (26)..(26): Xaa at position 26 is leucine or absent.  If leucine then Xaa in positions 27 through 31 are not absent.
  MISC_FEATURE (27)..(27): Xaa at position 27 is leucine or absent.  If leucine then Xaa in positions 28 through 31 are not absent.
  MISC_FEATURE (28)..(28): Xaa at position 28 is threonine, proline or absent.  If threonine or proline then Xaa in positions 29 through 31 are not absent.
  MISC_FEATURE (29)..(29): Xaa at position 29 is glutamic acid or absent. If glutamic acid then Xaa in positions 30 through 31 are not absent.
  MISC_FEATURE (30)..(30): Xaa at position 30 is valine or absent.  If valine then Xaa in position 31 is not absent.
  MISC_FEATURE (31)..(31): Xaa at position 31 is glutamic acid or aspartic acid or absent.
  MISC_FEATURE (33)..(33): Xaa at position 33 is proline, leucine or histidine.
  MISC_FEATURE (34)..(34): Xaa at position 34 is isoleucine or threonine
  MISC_FEATURE (36)..(36): Xaa at position 36 is asparagine or serine.
  MISC_FEATURE (37)..(37): Xaa at position 37 is glumatic acid or glycine.
  MISC_FEATURE (38)..(38): Xaa at position 38 is tryptophan or absent.
  MISC_FEATURE (39)..(39): Xaa at position 39 is glycine, glutamic acid or absent.  If glycine or glutamic acid then Xaa in position 38 is not absent.
  MISC_FEATURE (40)..(40): Xaa at position 40 is absent of present, if present Xaa in position 40 is cysteine, serine or alanine.  If Xaa in position 40 is present then positions 38 through 39 are not absent.
  MISC_FEATURE (41)..(41): Xaa at position 41 is arginine, lysine or absent.  If arginine or lysine than Xaa in positions 38 through 40 are not absent.
  MISC_FEATURE (42)..(42): Xaa at position 42 is absent or present, if present Xaa in position 42 is cysteine, serine or alanine.  If Xaa in position 42 is present then positions 38 through 41 are not absent.
  MISC_FEATURE (43)..(43): Xaa at position 43 is asparagine, serine, glycine or absent.  If asparagine or serine or glycine then Xaa in positions 38 through 42 are not absent.
  MISC_FEATURE (44)..(44): Xaa at position 44 is aspartic acid, glycine or absent.  If aspartic acid or glycine then Xaa in positions 38 through 43 are not absent.
  MISC_FEATURE (45)..(45): Xaa at position 45 is serine or absent.  If serine then Xaa in positions 38 through 44 are not absent.
  MISC_FEATURE (46)..(46): Xaa at position 46 is serine or absent.  If serine then Xaa in positions 38 through 45 are not absent.
  MISC_FEATURE (47)..(47): Xaa at position 47 is aspartic acid or absent. If aspartic acid then Xaa in positions 38 through 46 are not absent.
  MISC_FEATURE (48)..(48): Xaa at position 48 is serine or absent.  If serine then Xaa in positions 49 through 54 are not absent.
  MISC_FEATURE (49)..(49): Xaa at position 49 is leucine or absent.  If leucine then Xaa in positions 50 through 54 are not absent.
  MISC_FEATURE (50)..(50): Xaa at position 50 is leucine or absent.  If leucine then Xaa in positions 51 through 54 are not absent.
  MISC_FEATURE (51)..(51): Xaa at position 51 is threonine, proline or absent.  If threonine or proline then Xaa in positions 52 through 54 are not absent.
  MISC_FEATURE (52)..(52): Xaa at position 52 is glutamic acid or absent. If glutamic acid then Xaa in positions 53 through 54 are not absent.
  MISC_FEATURE (53)..(53): Xaa at position 53 is valine or absent.  If valine then Xaa in position 54 is not absent.
  MISC_FEATURE (54)..(54): Xaa at position 54 is glutamic acid or aspartic acid or absent
  MISC_FEATURE (56)..(56): Xaa at position 56 is proline, leucine or histidine.
  MISC_FEATURE (57)..(57): Xaa at position 57 is isoleucine or threonine.
  MISC_FEATURE (59)..(59): Xaa at position 59 is asparagine or serine.
  MISC_FEATURE (60)..(60): Xaa at position 60 is glumatic acid or glycine.
  MISC_FEATURE (61)..(61): Xaa at position 61 is tryptophan or absent.
  MISC_FEATURE (62)..(62): Xaa at position 62 is glycine, glutamic acid or absent.  If glycine or glutamic acid then Xaa in position 61 is not absent.
  MISC_FEATURE (63)..(63): Xaa at position 63 is absent of present, if present Xaa in position 63 is cysteine, serine or alanine.  If Xaa in position 63 is present then positions 61 through 62 are not absent.
  MISC_FEATURE (64)..(64): Xaa at position 64 is arginine, lysine or absent.  If arginine or lysine than Xaa in positions 61 through 63 are not absent.
  MISC_FEATURE (65)..(65): Xaa at position 65 is absent or present, if present Xaa in position 65 is cysteine, serine or alanine.  If Xaa in position 65 is present then positions 61 through 64 are not absent.
  MISC_FEATURE (66)..(66): Xaa at position 66 is asparagine, serine, glycine or absent.  If asparagine or serine or glycine then Xaa in positions 61 through 65 are not absent.
  MISC_FEATURE (67)..(67): Xaa at position 67 is aspartic acid, glycine or absent.  If aspartic acid or glycine then Xaa in positions 61 through 66 are not absent.
  MISC_FEATURE (68)..(68): Xaa at position 68 is serine or absent.  If serine then Xaa in positions 61 through 67 are not absent.
  MISC_FEATURE (69)..(69): Xaa at position 69 is serine or absent.  If serine then Xaa in positions 61 through 68 are not absent.
  MISC_FEATURE (70)..(70): Xaa at position 70 is aspartic acid or absent. If aspartic acid then Xaa in positions 61 through 69 are not absent.
XXXXXXXXTXXRXXXXXXXXXXXXXXXXXXXTXXRXXXXXXXXXXXXXXXXXXXTXXRXXXXXXXXXXXX

SEQ ID NO: 76 - protein, 17 aa, Influenza B virus
NNATFNYTNVNPISHIR

SEQ ID NO: 77 - protein, 6 aa, Influenza virus
MLEPFQ

SEQ ID NO: 78 - protein, 6 aa, Influenza virus
MLEPLQ

SEQ ID NO: 79 - protein, 142 aa, Yersinia pestis
DILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSD

SEQ ID NO: 80 - protein, 19 aa, Haemophilus influenzae
CSSSNNDAAGNGAAQFGGY

SEQ ID NO: 81 - protein, 11 aa, Haemophilus influenzae
NKLGTVSYGEE

SEQ ID NO: 82 - protein, 16 aa, Haemophilus influenzae
NDEAAYSKNRRAVLAY

SEQ ID NO: 83 - protein, 28 aa, Moraxella catarrhalis
LDIEKDKKKRTDEQLQAELDDKYAGKGY

SEQ ID NO: 84 - protein, 28 aa, Moraxella catarrhalis
LDIEKNKKKRTEAELQAELDDKYAGKGY

SEQ ID NO: 85 - protein, 28 aa, Moraxella catarrhalis
IDIEKKGKIRTEAELLAELNKDYPGQGY

SEQ ID NO: 86 - protein, 25 aa, Porphyromonas gingivalis
GVSPKVCKDVTVEGSNEFAPVQNLT

SEQ ID NO: 87 - protein, 20 aa, Porphyromonas gingivalis
RIQSTWRQKTVDLPAGTKYV

SEQ ID NO: 88 - protein, 21 aa, Trypanosoma cruzi
KAAIAPAKAAAAPAKAATAPA

SEQ ID NO: 89 - protein, 16 aa, PLASMODIUM FALCIPARUM
NANPNANPNANPNANP

SEQ ID NO: 90 - protein, 24 aa, Plasmodium falciparum
NANPNVDPNANPNANPNANPNVDP

SEQ ID NO: 91 - protein, 20 aa, Plasmodium falciparum
NANPNVDPNANPNANPNANP

SEQ ID NO: 92 - protein, 20 aa, Plasmodium falciparum
NANPNANPNANPNVDPNANP

SEQ ID NO: 93 - protein, 28 aa, Plasmodium falciparum
NANPNVDPNANPNANPNANPNVDPNANP

SEQ ID NO: 94 - protein, 20 aa, Plasmodium falciparum
NPNVDPNANPNANPNANPNV

SEQ ID NO: 95 - protein, 22 aa, Plasmodium falciparum
NPNVDPNANPNANPNANPNVDP

SEQ ID NO: 96 - protein, 24 aa, Plasmodium falciparum
NPNVDPNANPNANPNANPNVDPNA

SEQ ID NO: 97 - protein, 18 aa, Plasmodium falciparum
NVDPNANPNANPNANPNV

SEQ ID NO: 98 - protein, 20 aa, Plasmodium falciparum
NVDPNANPNANPNANPNVDP

SEQ ID NO: 99 - protein, 22 aa, Plasmodium falciparum
NVDPNANPNANPNANPNVDPNA

SEQ ID NO: 100 - protein, 16 aa, Plasmodium falciparum
DPNANPNANPNANPNV

SEQ ID NO: 101 - protein, 18 aa, Plasmodium falciparum
DPNANPNANPNANPNVDP

SEQ ID NO: 102 - protein, 20 aa, Plasmodium falciparum
DPNANPNANPNANPNVDPNA

SEQ ID NO: 103 - protein, 19 aa, Plasmodium vivax
GDRADGQPAGDRADGQPAG

SEQ ID NO: 104 - protein, 18 aa, Plasmodium vivax
RADDRAAGQPAGDGQPAG

SEQ ID NO: 105 - protein, 18 aa, Plasmodium vivax
ANGAGNQPGANGAGDQPG

SEQ ID NO: 106 - protein, 18 aa, Plasmodium vivax
ANGADNQPGANGADDQPG

SEQ ID NO: 107 - protein, 18 aa, Plasmodium vivax
ANGAGNQPGANGADNQPG

SEQ ID NO: 108 - protein, 18 aa, Plasmodium vivax
ANGAGNQPGANGADDQPG

SEQ ID NO: 109 - protein, 22 aa, Plasmodium vivax
APGANQEGGAAAPGANQEGGAA

SEQ ID NO: 110 - protein, 36 aa, Plasmodium vivax
ANGAGNQPGANGAGDQPGANGADNQPGANGADDQPG

SEQ ID NO: 111 - protein, 16 aa, Plasmodium berghei
DPPPPNPNDPPPPNPN

SEQ ID NO: 112 - protein, 24 aa, Plasmodium yoelii
QGPGAPQGPGAPQGPGAPQGPGAP

SEQ ID NO: 113 - protein, 15 aa, Streptococcus sobrinus
KPRPIYEAKLAQNQK

SEQ ID NO: 114 - protein, 16 aa, Streptococcus sobrinus
AKADYEAKLAQYEKDL

SEQ ID NO: 115 - protein, 9 aa, Shigella flexneri
KDRTLIEQK

SEQ ID NO: 116 - protein, 15 aa, respiratory syncytial virus
CSICSNNPTCWAICK

SEQ ID NO: 117 - protein, 25 aa, Entamoeba histolytica
VECASTVCQNDNSCPIIADVEKCNQ

SEQ ID NO: 118 - protein, 34 aa, Schistosoma japonicum
DLQSEISLSLENGELIRRAKSAESLASELQRRVD

SEQ ID NO: 119 - protein, 34 aa, Schistosoma mansoni
DLQSEISLSLENSELIRRAKAAESLASDLQRRVD

SEQ ID NO: 120 - protein, 26 aa, Bovine Inhibin
STPPLPWPWSPAALRLLQRPPEEPAA

SEQ ID NO: 121 - protein, 17 aa, Ebola virus
ATQVEQHHRRTDNDSTA

SEQ ID NO: 122 - protein, 17 aa, Ebola virus
HNTPVYKLDISEATQVE

SEQ ID NO: 123 - protein, 17 aa, Ebola virus
GKLGLITNTIAGVAVLI

SEQ ID NO: 124 - protein, 14 aa, Escherichia coli
CCELCCYPACAGCN

SEQ ID NO: 125 - protein, 18 aa, Escherichia coli
NTFYCCELCCYPACAGCN

SEQ ID NO: 126 - protein, 18 aa, Escherichia coli
SSNYCCELCCYPACAGCN

SEQ ID NO: 127 - protein, 42 aa, Alzheimer′s disease b-Amyloid [sequence omitted]

SEQ ID NO: 128 - protein, 17 aa, Alzheimer′s disease b-Amyloid
DAEFRHDSGYEVHHQKL

SEQ ID NO: 129 - protein, 11 aa, Alzheimer′s disease b-Amyloid
EDVGSNKGAII

SEQ ID NO: 130 - protein, 33 aa, Alzheimer′s disease b-Amyloid
DAEFRHDSGYEVHHQKLVFFAEDVGSNKGAIIG

SEQ ID NO: 131 - protein, 32 aa, alzheimer′s disease b-amplyoid
DAEFRHDSGYEVHHQKLVFFAEDVGSNKGAII

SEQ ID NO: 132 - protein, 13 aa, Neisseria meningitidis
YVAVENGVAKKVA

SEQ ID NO: 133 - protein, 15 aa, Neisseria meningitidis
HFVQQTPKSQPTLVP

SEQ ID NO: 134 - protein, 13 aa, Neisseria meningitidis
HVVVNNKVATHVP

SEQ ID NO: 135 - protein, 12 aa, Neisseria meningitidis
PLQNIQPQVTKR

SEQ ID NO: 136 - protein, 21 aa, Neisseria meningitidis
AQAANGGAASGQVKVTKVTKA

SEQ ID NO: 137 - protein, 10 aa, Neisseria meningitidis
YVDEQSKYHA

SEQ ID NO: 138 - protein, 15 aa, Neisseria meningitidis
HFVQNKQNQPPTLVP

SEQ ID NO: 139 - protein, 18 aa, Neisseria meningitidis
KPSSTNAKTGNKVEVTKA

SEQ ID NO: 140 - protein, 17 aa, Neisseria meningitidis
YWTTVNTGSATTTTFVP

SEQ ID NO: 141 - protein, 11 aa, Neisseria meningitidis
YVDEKKKMVHA

SEQ ID NO: 142 - protein, 13 aa, Neisseria meningitidis
HYTRQNNADVFVP

SEQ ID NO: 143 - protein, 14 aa, Neisseria meningitidis
YYTKDTNNNLTLVP

SEQ ID NO: 144 - protein, 14 aa, Neisseria meningitidis
PPQKNQSQPVVTKA

SEQ ID NO: 145 - protein, 14 aa, Neisseria meningitidis
PPSKGQTGNKVTKG

SEQ ID NO: 146 - protein, 14 aa, Neisseria meningitidis
PPSKSQPQVKVTKA

SEQ ID NO: 147 - protein, 18 aa, Neisseria meningitidis
QPQTANTQQGGKVKVTKA

SEQ ID NO: 148 - protein, 18 aa, Neisseria meningitidis
QPQVTNGVQGNQVKVTKA

SEQ ID NO: 149 - protein, 18 aa, Neisseria meningitidis
QPSKAQGQTNNQVKVTKA

SEQ ID NO: 150 - protein, 20 aa, Neisseria meningitidis
PPSSNQGKNQAQTGNTVTKA

SEQ ID NO: 151 - protein, 18 aa, Neisseria meningitidis
PPSKSQGKTGNQVKVTKA

SEQ ID NO: 152 - protein, 18 aa, Neisseria meningitidis
PPSKSQGTNNNQVKVTKA

SEQ ID NO: 153 - protein, 18 aa, Neisseria meningitidis
PPSKSQPGQVKVTKVTKA

SEQ ID NO: 154 - protein, 24 aa, Neisseria meningitidis
QLQLTEQPSSTNGQTGNQVKVTKA

SEQ ID NO: 155 - protein, 24 aa, Neisseria meningitidis
QLQLTEAPSKSQGAASNQVKVTKA

SEQ ID NO: 156 - protein, 19 aa, Neisseria meningitidis
SAYTPAHVYVDNKVAKHVA

SEQ ID NO: 157 - protein, 21 aa, Neisseria meningitidis
SAYTPAHFVQNKQNNNPTLVP

SEQ ID NO: 158 - protein, 12 aa, Neisseria meningitidis
VEGRNYQLQLTE

SEQ ID NO: 159 - protein, 12 aa, Neisseria meningitidis
PAQNSKSAYTPA

SEQ ID NO: 160 - protein, 22 aa, Neisseria meningitidis
QLQLTEPPSKNQAQTQNKVTKA

SEQ ID NO: 161 - protein, 16 aa, Neisseria meningitidis
GRDAFELFLLGSGSDE

SEQ ID NO: 162 - protein, 31 aa, Neisseria meningitidis
RHANVGRDAFELFLLGSGSDEAKGTDPLKNH

SEQ ID NO: 163 - protein, 18 aa, Neisseria meningitidis
GRDAFNLFLLGRIGDDDE

SEQ ID NO: 164 - protein, 17 aa, Neisseria meningitidis
GRNAFELFLIGSATSDQ

SEQ ID NO: 165 - protein, 15 aa, Neisseria meningitidis
QVKVTKAKSRIRTKI

SEQ ID NO: 166 - protein, 13 aa, Neisseria meningitidis
TLVPAVVGKPGSD

SEQ ID NO: 167 - protein, 17 aa, Neisseria meningitidis
HAKASSSLGSAKGFSPR

SEQ ID NO: 168 - protein, 15 aa, Neisseria meningitidis
TRYKNYKAPSTDFKL

SEQ ID NO: 169 - protein, 18 aa, Neisseria meningitidis
SLNRASVDLGGSDSFSQT

SEQ ID NO: 170 - protein, 21 aa, Neisseria meningitidis
GKVNTVKNVRSGELSAGVRVK

SEQ ID NO: 171 - protein, 21 aa, Neisseria meningitidis
GKVNTVKNVRSGELSVGVRVK

SEQ ID NO: 172 - protein, 13 aa, Homo sapiens
APEWPGSRDKRTL

SEQ ID NO: 173 - protein, 9 aa, Homo sapiens
EDGQVMDVD

SEQ ID NO: 174 - protein, 8 aa, Homo sapiens
STTQEGEL

SEQ ID NO: 175 - protein, 10 aa, Homo sapiens
GHTFEDSTKK

SEQ ID NO: 176 - protein, 8 aa, Homo sapiens
GGGHFPPT

SEQ ID NO: 177 - protein, 6 aa, Homo sapiens
PGTINI

SEQ ID NO: 178 - protein, 5 aa, Homo sapiens
FTPPT

SEQ ID NO: 179 - protein, 8 aa, Homo sapiens
INHRGYWV

SEQ ID NO: 180 - protein, 17 aa, Homo sapiens
GEFCINHRGYWVCGDPA

SEQ ID NO: 181 - protein, 14 aa, Homo sapiens
MAPEWPGSRDKRTL

SEQ ID NO: 182 - protein, 10 aa, Homo sapiens
MEDGQVMDVD

SEQ ID NO: 183 - protein, 9 aa, Homo sapiens
MSTTQEGEL

SEQ ID NO: 184 - protein, 11 aa, Homo sapiens
MGHTFEDSTKK

SEQ ID NO: 185 - protein, 9 aa, Homo sapiens
MGGGHFPPT

SEQ ID NO: 186 - protein, 7 aa, Homo sapiens
MPGTINI

SEQ ID NO: 187 - protein, 6 aa, Homo sapiens
MFTPPT

SEQ ID NO: 188 - protein, 9 aa, Homo sapiens
MINHRGYWV

SEQ ID NO: 189 - protein, 18 aa, Homo sapiens
MGEFCINHRGYWVCGDPA

SEQ ID NO: 190 - protein, 27 aa, HEPATITIS B
LQAGFFLLTRILTIPQSLDSWWTSLNF

SEQ ID NO: 191 - protein, 21 aa, Hepatitis B virus
MGTNLSVPNPLGFFPDHQLDP

SEQ ID NO: 192 - protein, 8 aa, Hepatitis B virus
PLGFFPDH

SEQ ID NO: 193 - protein, 10 aa, Hepatitis B virus
PLGFFPDHQL

SEQ ID NO: 194 - protein, 26 aa, Hepatitis B virus
MQWNSTAFHQTLQDPRVRGLYLPAGG

SEQ ID NO: 195 - protein, 14 aa, Hepatitis B
MQWSTAFHQTLQDP

SEQ ID NO: 196 - protein, 14 aa, Hepatitis B virus
MQWSTALHQALQDP

SEQ ID NO: 197 - protein, 6 aa, Hepatitis B virus
QDPRVR

SEQ ID NO: 198 - protein, 13 aa, Hepatitis B virus
DPRVRGLYLPAGG

SEQ ID NO: 199 - protein, 13 aa, Hepatitis B virus
DPRVRGLYFPAGG

SEQ ID NO: 200 - protein, 24 aa, B. anthracis
IVTKENTIINPSENGDTSTNGIEL

SEQ ID NO: 201 - protein, 15 aa, Hookworm
IVYQHSHGEDRPGEL

SEQ ID NO: 202 - protein, 11 aa, VESICULAR STOMATITIS VIRUS
YTDIEMNRLGK

SEQ ID NO: 203 - protein, 26 aa, BOVINE RESPIRATORY SYNCYTIAL VIRUS
DKELLPKVNNHDCQISNIATVIEFQQ

SEQ ID NO: 204 - protein, 26 aa, HUMAN RESPIRATORY SYNCYTIAL VIRUS
DKQLLPIVNKQSCSISNIETVIEFQQ

SEQ ID NO: 205 - protein, 26 aa, HUMAN RESPIRATORY SYNCYTIAL VIRUS
DKRLLPIVNQQSCRISNIETVIEFQQ

SEQ ID NO: 206 - protein, 6 aa, HUMAN RESPIRATORY SYNCYTIAL VIRUS
FPSDEF

SEQ ID NO: 207 - protein, 14 aa, HUMAN RHINOVIRUS
PATGIDNHREAKLD

SEQ ID NO: 208 - protein, 10 aa, POLIO VIRUS
SASTKNKDKL

SEQ ID NO: 209 - protein, 16 aa, HIV P24
GPKEPFRDYVDRFYKC

SEQ ID NO: 210 - protein, 17 aa, CORYNEBACTERIUM DIPTHERIAE TOXIN
FQVVHNSYNRPAYSPGC

SEQ ID NO: 211 - protein, 25 aa, BORRELIA BURGDORFERI
VEIKEGTVTLKREIDKNGKVTVSLC

SEQ ID NO: 212 - protein, 19 aa, BORRELIA BURGDORFERI
TLSKNISKSGEVSVELNDC

SEQ ID NO: 213 - protein, 11 aa, INFLUENZA VIRUS
SSVSSFERFEC

SEQ ID NO: 214 - protein, 10 aa, INFLUENZA VIRUS
LIDALLGDPC

SEQ ID NO: 215 - protein, 9 aa, INFLUENZA VIRUS
TLIDALLGC

SEQ ID NO: 216 - protein, 24 aa, INFLUENZA VIRUS
FWRGENGRKTRSAYERMCNILKGK

SEQ ID NO: 217 - protein, 22 aa, INFLUENZA VIRUS
LRVLSFIRGTKVSPRGKLSTRG

SEQ ID NO: 218 - protein, 22 aa, INFLUENZA VIRUS
SLVGIDPFKLLQNSQVYSLIRP

SEQ ID NO: 219 - protein, 24 aa, INFLUENZA VIRUS
AVKGVGTMVMELIRMIKRGINDRN

SEQ ID NO: 220 - protein, 21 aa, TRYPANOSOMA CRUZI
SHNFTLVASVIIEEAPSGNTC

SEQ ID NO: 221 - protein, 16 aa, PLASMODIUM FALCIPARUM MSP1 1
SVQIPKVPYPNGIVYC

SEQ ID NO: 222 - protein, 16 aa, PLASMODIUM FALCIPARUM
DFNHYYTLKTGLEADC

SEQ ID NO: 223 - protein, 18 aa, PLASMODIUM FALCIPARUM
PSDKHIEQYKKIKNSISC

SEQ ID NO: 224 - protein, 20 aa, PLASMODIUM FALCIPARUM
EYLNKIQNSLSTEWSPCSVT

SEQ ID NO: 225 - protein, 19 aa, P VIVAX
YLDKVRATVGTEWTPCSVT

SEQ ID NO: 226 - protein, 20 aa, P YOELII
EFVKQISSQLTEEWSQCSVT

SEQ ID NO: 227 - protein, 16 aa, STREPTOCOCCUS SOBRINUS
KPRPIYEAKLAQNQKC

SEQ ID NO: 228 - protein, 17 aa, STREPTOCOCCUS SOBRINUS
AKADYEAKLAQYEKDLC

SEQ ID NO: 229 - protein, 16 aa, LYMPHOCYTIC CHORIOMENINGITIS VIRUS
RPQASGVYMGNLTAQC

SEQ ID NO: 230 - protein, 16 aa, CLOSTRIDIUM TETANI
QYIKANSKFIGITELC

SEQ ID NO: 231 - protein, 19 aa, Neisseria meningitidis
AIWQVEQKASIAGTDSGWC

SEQ ID NO: 232 - protein, 19 aa, Neisseria meningitidis
NYKNGGFFVQYGGAYKRHC

SEQ ID NO: 233 - protein, 19 aa, Neisseria meningitidis
HNSQTEVAATLAYRFGNVC

SEQ ID NO: 234 - protein, 19 aa, Neisseria meningitidis
TPRVSYAHGFKGLVDDADC

SEQ ID NO: 235 - protein, 19 aa, Neisseria meningitidis
RFGNAVPRISYAHGFDFIC

SEQ ID NO: 236 - protein, 19 aa, Neisseria meningitidis
AFKYARHANVGRNAFELFC

SEQ ID NO: 237 - protein, 20 aa, Neisseria meningitidis
SGAWLKRNTGIGNYTQINAC

SEQ ID NO: 238 - protein, 16 aa, Neisseria meningitidis
AGEFGTLRAGRVANQC

SEQ ID NO: 239 - protein, 16 aa, Neisseria meningitidis
IGNYTQINAASVGLRC

SEQ ID NO: 240 - protein, 16 aa, Neisseria meningitidis
GRNYQLQLTEQPSRTC

SEQ ID NO: 241 - protein, 16 aa, Neisseria meningitidis
SGSVQFVPAQNSKSAC

SEQ ID NO: 242 - protein, 16 aa, Neisseria meningitidis
HANVGRDAFNLFLLGC

SEQ ID NO: 243 - protein, 16 aa, Neisseria meningitidis
LGRIGDDDEAKGTDPC

SEQ ID NO: 244 - protein, 16 aa, Neisseria meningitidis
SVQFVPAQNSKSAYKC

SEQ ID NO: 245 - protein, 16 aa, Neisseria meningitidis
NYAFKYAKHANVGRDC

SEQ ID NO: 246 - protein, 16 aa, Neisseria meningitidis
AHGFDFIERGKKGENC

SEQ ID NO: 247 - protein, 16 aa, Neisseria meningitidis
GVDYDFSKRTSAIVSC

SEQ ID NO: 248 - protein, 16 aa, Neisseria meningitidis
HDDMPVSVRYDSPDFC

SEQ ID NO: 249 - protein, 27 aa, Neisseria meningitidis
RFGNAVPRISYAHGFDFIERGKKGENC

SEQ ID NO: 250 - protein, 24 aa, Neisseria meningitidis
NYAFKYAKHANVGRDAFNLFLLGC

SEQ ID NO: 251 - protein, 26 aa, Neisseria meningitidis
SGAWLKRNTGIGNYTQINAASVGLRC

SEQ ID NO: 252 - protein, 20 aa, Neisseria meningitidis
SGSVQFVPAQNSKSAYTPAC

SEQ ID NO: 253 - protein, 19 aa, Neisseria meningitidis
TGANNTSTVSDYFRNRITC

SEQ ID NO: 254 - protein, 19 aa, Neisseria meningitidis
IYDFKLNDKFDKFKPYIGC

SEQ ID NO: 255 - protein, 19 aa, Neisseria meningitidis
LSAIYDFKLNDKFKPYIGC

SEQ ID NO: 256 - protein, 19 aa, Neisseria meningitidis
NGWYINPWSEVKFDLNSRC

SEQ ID NO: 257 - protein, 21 aa, HEPATITIS B
MGTNLSVPNPLGFFPDHQLDP

SEQ ID NO: 258 - protein, 8 aa, HEPATITIS B
PLGFFPDH

SEQ ID NO: 259 - protein, 11 aa, HEPATITIS B
PLGFFPDHQLH

SEQ ID NO: 260 - protein, 26 aa, HEPATITIS B
MQWNSTAFHQTLQDPRVRGLYLPAGG

SEQ ID NO: 261 - protein, 14 aa, HEPATITIS B
MQWSTAFHQTLQDP

SEQ ID NO: 262 - protein, 15 aa, HEPATITIS B
MQWNSTALHQALQDP

SEQ ID NO: 263 - protein, 6 aa, HEPATITIS B
QDPRVR

SEQ ID NO: 264 - protein, 20 aa, Hepatitis B virus
MDIDPYKEFGATVELLSFLP

SEQ ID NO: 265 - protein, 24 aa, Hepatitis B virus
RDLLDASALYREALESPEHCSPHH

SEQ ID NO: 266 - protein, 25 aa, Hepatitis B virus
TWVGVNLEDPASRDLVVSYVNTNMG

SEQ ID NO: 267 - protein, 16 aa, Hepatitis B virus
VVSYVNTNMGLKFRQL

SEQ ID NO: 268 - protein, 21 aa, Hepatitis B virus
LLWFHISCLTFGRETVIEYLV

SEQ ID NO: 269 - protein, 32 aa, Hepatitis B virus
LLWFHISCLTFVSFGVWIRTPPAYRPPNAPIL

SEQ ID NO: 270 - protein, 21 aa, Hepatitis B virus
VSFGVWIRTPPAYRPPNAPIL

SEQ ID NO: 271 - protein, 12 aa, Hepatitis B virus
VSFGVWIRTPPA

SEQ ID NO: 272 - protein, 12 aa, Hepatitis B virus
PPAYRPPNAPIL

SEQ ID NO: 273 - protein, 12 aa, Hepatitis B virus
WIRTPPAYRPPN

SEQ ID NO: 274 - protein, 20 aa, Hepatitis B virus
PHHTALRQAILCWGELMTLA

SEQ ID NO: 275 - protein, 13 aa, ARTIFICIAL SEQUENCE
  T CELL EPITOPE
AKFVAAWTLKAAA

SEQ ID NO: 276 - protein, 14 aa, ARTIFICIAL SEQUENCE
  BIOTIN BINDING EPITOPE
GGGCSWAPPFKASC

SEQ ID NO: 277 - protein, 16 aa, ARTIFICIAL SEQUENCE
  BIOTIN BIDNING EPITOPE
GGGRGEFTGTYITAVT

SEQ ID NO: 278 - DNA, 24 bp, Hepatitis B virus
ttgggccatggacatcgaccctta

SEQ ID NO: 279 - DNA, 42 bp, Artificial Sequence
  Amplification primer containing a restriction endonuclease site
cgcaagcttagagctcttgaattccaacaacagtagtctccg

SEQ ID NO: 280 - DNA, 78 bp, Influenza A virus; identical to SEQ ID NO: 9

SEQ ID NO: 281 - DNA, 70 bp, Influenza A virus
cgtcagagctatcattagagcggctaccccattcgttacgaatcggcgtctccacttcggttaacagaga

SEQ ID NO: 282 - protein, 33 aa, ARTIFICIAL SEQUENCE; identical to SEQ ID NO: 23
  LINKER TO HBC PARTICLE

SEQ ID NO: 283 - protein, 53 aa, ARTIFICIAL SEQUENCE
  LYSINE LINKER
NRNAVPNLRGDLQVLAQKVARTLPTSNRNAVPNLRGDLQVLAQKVARTLPTSC

SEQ ID NO: 284 - protein, 53 aa, ARTIFICIAL SEQUENCE
  LYSINE LINKER
YSASGSGVRGDLGSLAPRVARQLPASYSASGSGVRGDLGSLAPRVARQLPASC

SEQ ID NO: 285 - protein, 23 aa, ARTIFICIAL SEQUENCE
  IMMUNOGENIC SEQUENCE OF THE FMDV EPITOPE
RGDLQVRGDLQVLAQKVARTLPC

SEQ ID NO: 286 - protein, 29 aa, ARTIFICIAL SEQUENCE
  IMMUNOGENIC SEQUENCE OF FMDV EPITOPE
RGDLQVRGDLQVRGDLQVLAQKVARTLPC

SEQ ID NO: 287 - protein, 8 aa, ARTIFICIAL SEQUENCE
  LINKER EXTENSION
SASTKNKD

SEQ ID NO: 288 - protein, 10 aa, ARTIFICIAL SEQUENCE
  LINKER EXTENSION
SASTKNKDKL

SEQ ID NO: 289 - protein, 8 aa, ARTIFICIAL SEQUENCE
  LINKER EXTENSION
PASTTNKD

SEQ ID NO: 290 - protein, 10 aa, ARTIFICIAL SEQUENCE
  LINKER EXTENSION
PASTTNKDKL

SEQ ID NO: 291 - protein, 20 aa, ARTIFICIAL SEQUENCE
  PEPTIDE WITH MUTATION
PNLRGDLQVLAQKVARTLPC

SEQ ID NO: 292 - protein, 15 aa, ARTIFICIAL SEQUENCE
  NIMII EPITOPE
VKAETRLNPDLQPTC

SEQ ID NO: 293 - protein, 15 aa, ARTIFICIAL SEQUENCE
  PEPTIDE
QDPRVRGLYFPAGGC

SEQ ID NO: 294 - protein, 20 aa, ARTIFICIAL SEQUENCE
  HPV16 L1 PROTEIN
PNDTFIVSTNPNTVTSSTPI

SEQ ID NO: 295 - protein, 33 aa, ARTIFICIAL SEQUENCE; identical to SEQ ID NO: 10
  LEUCINE ZIPPER OF GCN4 PROTEIN

SEQ ID NO: 296 - protein, 33 aa, ARTIFICIAL SEQUENCE; identical to SEQ ID NO: 23
  GCN4-VL LEUCINE ZIPPER

SEQ ID NO: 297 - protein, 25 aa, ARTIFICIAL SEQUENCE
  HUMAN AP4 LEUCINE ZIPPER
IFSLEQEKTRLLQQNTQLKRFIQEL

SEQ ID NO: 298 - protein, 33 aa, ARTIFICIAL SEQUENCE; identical to SEQ ID NO: 23
  ZIPPER

SEQ ID NO: 299 - protein, 13 aa, ARTIFICIAL SEQUENCE
  HPV 16 L2
LVEETSFIDAGAP

SEQ ID NO: 300 - protein, 32 aa, ARTIFICIAL SEQUENCE; identical to SEQ ID NO: 13
  C-MYC LEUCINE ZIPPER

SEQ ID NO: 301 - protein, 26 aa, ARTIFICIAL SEQUENCE
  TMV 89-114
DTRNRIIEVENQQSPTTAETLDATRR

SEQ ID NO: 302 - protein, 159 aa, ARTIFICIAL SEQUENCE
  TMV L CP
MSYSITSPSQFVFLSSVWADPIELLNVCTNSLGNQFQTQQARTTVQQQFSEVWKPFPQSTVRFPGDVYKVYRYNAVLDPLITALLGAFDTRNRIIEVENQQSPTTAETLDATRRVDDATVAIRSAINNLVNELVRGTGLYNQNTFESMSGLVWTSAPAS

SEQ ID NO: 303 - protein, 10 aa, ARTIFICIAL SEQUENCE
  SABIN STRAIN OF POLIOVIRUS
SASTKNKDKL

SEQ ID NO: 304 - protein, 24 aa, ARTIFICIAL SEQUENCE
  COMP PEPTIDE
LKFRFRDIERSKRSVMVGHTATAA

SEQ ID NO: 305 - protein, 16 aa, ARTIFICIAL SEQUENCE
  YEAST TY1 CAPSID
GELAEQKLEQALQKLA

SEQ ID NO: 306 - protein, 33 aa, ARTIFICIAL SEQUENCE; identical to SEQ ID NO: 24
  GCN4 LL

SEQ ID NO: 307 - protein, 20 aa, ARTIFICIAL SEQUENCE
  C-TERMINUS OF P1
GGGGGGLAQKLLEALQKALA

SEQ ID NO: 308 - protein, 20 aa, ARTIFICIAL SEQUENCE
  HERRINGBONE STRUCTURE
GGGGGGLAQKLLEALQKALA

SEQ ID NO: 309 - protein, 20 aa, ARTIFICIAL SEQUENCE
  CHIMERIC CAPSID PROTEIN
VPNLRGDLQVLAQKVARTLP

SEQ ID NO: 310 - protein, 11 aa, ARTIFICIAL SEQUENCE
  CHIMERIC CAPSID PROTEIN CONSTRUCT PIECE
YTDIEMNRLGK

SEQ ID NO: 311 - protein, 26 aa, ARTIFICIAL SEQUENCE
  VIRAL STRAIN RB94
DKELLPKVNNHDCQISNIATVIEFQQ

SEQ ID NO: 312 - protein, 26 aa, RSV
DKQLLPIVNKQSCSISNIETVIEFQQ

SEQ ID NO: 313 - protein, 26 aa, ARTIFICIAL SEQUENCE
  RSV SUBTYPE B
DKRLLPIVNQQSCRISNIETVIEFQQ

SEQ ID NO: 314 - protein, 6 aa, ARTIFICIAL SEQUENCE
  RSS-2 BRSV
FPSDEF

SEQ ID NO: 315 - protein, 14 aa, ARTIFICIAL SEQUENCE
  PRES2
QDPRVRGLYFPAGG

SEQ ID NO: 316 - protein, 27 aa, ARTIFICIAL SEQUENCE
  HBSAG
LQAGFFLLTRILTIPQSLDSWWTSLNF

SEQ ID NO: 317 - protein, 20 aa, ARTIFICIAL SEQUENCE
  STREP M P145
LRRDLDASREAKKQVEKALE

SEQ ID NO: 318 - protein, 42 aa, ARTIFICIAL SEQUENCE; identical to SEQ ID NO: 35
  V3 LOOP HIV1

SEQ ID NO: 319 - protein, 42 aa, ARTIFICIAL SEQUENCE; identical to SEQ ID NO: 36
  HIV1 V3 LOOP

SEQ ID NO: 320 - protein, 41 aa, ARTIFICIAL SEQUENCE; identical to SEQ ID NO: 37
  HIV1 V3 LOOP

SEQ ID NO: 321 - protein, 39 aa, ARTIFICIAL SEQUENCE; identical to SEQ ID NO: 38
  HIV1 V3 LOOP

SEQ ID NO: 322 - protein, 41 aa, ARTIFICIAL SEQUENCE; identical to SEQ ID NO: 39
  HIV1 V3 LOOP

SEQ ID NO: 323 - protein, 40 aa, ARTIFICIAL SEQUENCE; identical to SEQ ID NO: 40
  HIV1 V3 LOOP

SEQ ID NO: 324 - protein, 40 aa, ARTIFICIAL SEQUENCE; identical to SEQ ID NO: 41
  HIV1 V3 LOOP

SEQ ID NO: 325 - protein, 42 aa, ARTIFICIAL SEQUENCE; identical to SEQ ID NO: 42
  HIV1 V3 LOOP

SEQ ID NO: 326 - protein, 41 aa, ARTIFICIAL SEQUENCE; identical to SEQ ID NO: 43
  HIV1 V3 LOOP

SEQ ID NO: 327 - protein, 19 aa, ARTIFICIAL SEQUENCE
  HIV1 GP41
DRPEGIEEEGGERDRDRSD

SEQ ID NO: 328 - protein, 14 aa, ARTIFICIAL SEQUENCE
  VP1 OF HUMAN RHINOVIRUS 14
PATGIDNHREAKLD

SEQ ID NO: 329 - protein, 24 aa, ARTIFICIAL SEQUENCE
  T CELL EPITOPE PLASMODIUM FALCIPARUM
LTMSNVKNVSQTNFKSLLRNLGVS

SEQ ID NO: 330 - protein, 9 aa, ARTIFICIAL SEQUENCE
  INSERTION INTO M2
YPYDVPDYA

SEQ ID NO: 331 - protein, 9 aa, ARTIFICIAL SEQUENCE
  IGE
FVFFGSKTK

SEQ ID NO: 332 - protein, 12 aa, ARTIFICIAL SEQUENCE
  MAL INSERTION INTO M2
NANPNANPNANP

SEQ ID NO: 333 - protein, 20 aa, ARTIFICIAL SEQUENCE
  HPV L1 INSERTION INTO M2
PNDTFIVSTNPNTVTSSTPI

SEQ ID NO: 334 - protein, 20 aa, ARTIFICIAL SEQUENCE
  HPV INSERTION INTO M2
KGSPCTNVAVNPGDCPPLDL

SEQ ID NO: 335 - protein, 24 aa, ARTIFICIAL SEQUENCE
  HIV GP120 INSERTION INTO M2
NNTRKSIRIQRGPGRAFVTIGKIG

SEQ ID NO: 336 - protein, 22 aa, ARTIFICIAL SEQUENCE
  PSEUDOMONAS AERUGINOSA B AND T CELL EPITOPE
GTIALKPDPADGTADITLTFTM

SEQ ID NO: 337 - protein, 14 aa, ARTIFICIAL SEQUENCE
  BIOTIN BINDING PEPTIDE
GGGCSWAPPFKASC

SEQ ID NO: 338 - protein, 16 aa, ARTIFICIAL SEQUENCE
  BIOTIN BINDING PEPTIDE
GGGRGEFTGTYITAVT